(19) United States
(12) Patent Application Publication
(10) Pub. No.: US 2024/0218046 A1
(43) Pub. Date: Jul. 4, 2024

(54) FC-DERIVED POLYPEPTIDES

(71) Applicant: Anjarium Biosciences AG, Schlieren (CH)

(72) Inventors: Joel de Beer, Zollikon (CH); Monique Maurer, Zollikon (CH); Nicolas Meier, Thalwil (CH); Lavaniya Kunalingam, Zurich (CH); Marcello Clerici, Zurich (CH)

(21) Appl. No.: 18/555,437

(22) PCT Filed: Apr. 13, 2022
(86) PCT No.: PCT/EP2022/059902

§ 371 Date: Oct. 13, 2023

Related U.S. Application Data
(60) Provisional application No. 63/174,855, filed on Apr. 14, 2021.

(51) Int. Cl. C07K 14/715; C07K 16/28

(52) U.S. Cl. CPC C07K 14/715; C07K 16/283; C07K 2317/524; C07K 2317/526; C07K 2317/732; C07K 2317/734; C07K 2317/92; C07K 2319/03; C07K 2319/30; C07K 2319/33

(57) ABSTRACT

The present disclosure pertains to polypeptides comprising a transmembrane domain and an FcRn binding site (e.g., a modified Fc domain) and nanovesicles (e.g, extracellular vesicles (EVs) and hybridosomes) comprising such polypeptides. Said polypeptides can facilitate isolation and purification of nanovesicles comprising such polypeptides. The polypeptides and nanovesicles can be used in therapeutic and/or diagnostic applications. Also provided are nucleic acids and expression vectors encoding such polypeptides as well as cells expressing said polypeptides. Further provided are methods for producing nanovesicles comprising such polypeptides and methods for purifying these nanovesicles. Compositions comprising such polypeptides or nanovesicles as well as their uses are also described.

### PRIORITY

This application claims the benefit of priority to U.S. Ser. No. 63/174,855 filed Apr. 14, 2021, which is incorporated herein by reference in its entirety.

### REFERENCE TO SEQUENCE LISTING SUBMITTED ELECTRONICALLY

This application incorporates by reference a Sequence Listing submitted with this application as text file entitled “14497-004-228_Sequence_Listing.txt” created on Apr. 11, 2022 and having a size of 176,537 bytes.

### 1. FIELD

The present disclosure pertains to polypeptides comprising a transmembrane domain and an FcRn binding site (e.g., a modified Fc domain) and nanovesicles (e.g., extracellular vesicles (EVs) and hybridosomes) comprising such polypeptides. Said polypeptides can facilitate isolation and purification of nanovesicles comprising such polypeptides. The polypeptides and nanovesicles can be used in therapeutic and/or diagnostic applications. Also provided are nucleic acids and expression vectors encoding such polypeptides as well as cells expressing said polypeptides. Further provided are methods for producing nanovesicles comprising such polypeptides and methods for purifying these nanovesicles. Compositions comprising such polypeptides or nanovesicles as well as their uses are also described.

### 2. BACKGROUND OF THE DISCLOSURE

Despite major breakthroughs in the identification of new promising drug candidates, translating these findings into the clinic is often hampered by challenges in delivering an efficacious drug dosage to the site of the disease. A recently discovered cell-to-cell communication pathway may provide the missing puzzle piece for more precise drug delivery. It has emerged that almost all the cells within our body can establish links to neighboring as well as distant cells by the release of tiny “balloons”, termed extracellular vesicles (EVs). The discovery that these EVs, in particular exosomes, are functional shuttles of signaling molecules, inheritably led to the proposition that they could pose as ideal nanoscale candidates for drug delivery systems of modern-day pharmaceuticals. However, this notion is linked to several challenges, including with regard to preparing and isolating EVs as well as increasing their half life in circulation. Accordingly, suitable methods and compositions involved in generating, isolating and purifying EVs are needed to better enable therapeutic use and other applications of EV-based technologies.

There is therefore a need for improved methods of preparing membrane vesicles, suitable with industrial constraints and allowing production of vesicle preparations of therapeutic quality. To that end, International Patent Application Publication No. WO2019/081474 discloses a chromatographic techniques for capturing EVs genetically engineered to comprise Fc-binding polypeptides by using the Fc domains of antibodies bound to a chromatographic matrix and triggering elution of the captured EVs by lowering the pH below 8, or preferably below 6. However, significant improvement over said method is needed, especially as the EV therapeutics field advances toward clinical translation and impact of EV-based therapies.

Citation of a reference herein shall not be construed as an admission that such is prior art to the present disclosure.

### 3. SUMMARY OF THE DISCLOSURE

In one aspect, provided herein is a polypeptide, wherein the polypeptide comprises: a. a transmembrane domain; and b. a modified Fc domain of an immunoglobulin that i. is capable of specifically binding to the Fc binding site of an FcRn; and ii. lacks the ability to form homodimers.

In certain embodiments, the equilibrium dissociation constant of the modified Fc domain bound to FcRn at a pH of 6.5 has a value of at most 10⁻⁴M. In certain embodiments, the equilibrium dissociation constant of the modified Fc domain bound to FcRn at a pH of 7.4 has a value of at least 10⁻⁴ M.

In certain embodiments, the modified Fc domain is capable of specifically binding to the amino acid sequence between position 135-158 of human FcRn (SEQ ID NO: 7) and/or mouse FcRn (SEQ ID NO: 8). In certain embodiments, the modified Fc domain is capable of specifically binding to the amino acid sequence LNGEEFMX₁FX₂X₃X₄X₅GX₆WX₇GX₈W (SEQ ID NO:6), wherein X₁, X₂, X₃, X₄, X₅, X₆, X₇ and X₈ each is any amino acid.

In certain embodiments, said polypeptide does not substantially bind to C1q, FcγRI, FcγRII or FcγRIII.

In certain embodiments, the complement dependent cytotoxicity (CDC) activity of the modified Fc domain, the antibody dependent cell mediated cytotoxicity (ADCC) activity of the modified Fc domain, the antibody dependent cell mediated phagocytosis (ADCP) activity of the modified Fc domain, and/or the antibody dependent intracellular neutralization (ADIN) activity of the modified Fc domain, is decreased by at least 10%, 20%, 30%, 40%, or 50% compared to the unmodified Fc domain.

In certain embodiments, the complement dependent cytotoxicity (CDC) activity of the modified Fc domain, the antibody dependent cell mediated cytotoxicity (ADCC) activity of the modified Fc domain, the antibody dependent cell mediated phagocytosis (ADCP) activity of the modified Fc domain, and/or the antibody dependent intracellular neutralization (ADIN) activity of the modified Fc domain, is decreased by at least 1.5, 2, 3, 4, or 5-fold, compared to the unmodified Fc domain.

In certain embodiments, the FcRn binding polypeptide comprises from N-terminus to C-terminus: a. a modified CH2 domain that is modified relative to the unmodified CH2 domain to decrease effector function; b. a modified CH3 domain that is modified relative to the unmodified CH3 domain to lack the homodimerize; c. a linker sequence; and d. a transmembrane domain.

In certain embodiments, the FcRn binding polypeptide comprises from C-terminus to N-terminus: a. a modified CH3 domain that is modified relative to the unmodified CH3 domain to lack the homodimerize; b. a modified CH2 domain that is modified relative to the unmodified CH2 domain to decrease effector function; c. a linker sequence; and d. a transmembrane domain.

In various embodiments, the transmembrane domain is a multipass transmembrane domain.

In specific embodiments, the polypeptide further comprises a targeting domain selected from the group consisting of: scFv, (scFv)2, Fab, Fab′, F(ab′)2, F(abl)2, Fv, dAb, Fd fragments, diabodies, F(ab)2, F(ab′), F(ab′)3, Fd, Fv, disulfide linked Fv, dAb, sdAb, nanobody, CDR, di-scFv, bi-scFv, tascFv (tandem scFv), AVIBODY (e.g., diabody, triabody, tetrabody), T-cell engager (BiTE), V-NAR domain, Fcab, IgGACH2, DVD-Ig, probody, intrabody, DARPin, Centyrin, affibody, affilin, affitin, anticalin, avimer, Fynomer, Kunitz domain peptide, monobody, adnectin, tribody, and nanofitin.

In another aspect, provided herein is a nucleic acid encoding a polypeptide described herein.

In another aspect, provided herein is an expression vector comprising a nucleic acid described herein.

In another aspect, provided herein is a cell comprising a nucleic acid described herein or an expression vector described herein.

In another aspect, provided herein is an extracellular vesicle comprising a polypeptide described herein.

In another aspect, provided herein is a hybridosome comprising a polypeptide described herein.

In another aspect, provided herein is a method for purifying an extracellular vesicle (EV), wherein said method comprises: a. providing the EV wherein the EV is associated with a first binding partner, wherein the first binding partner is capable of binding to the Fc binding site of an FcRn in a pH dependent manner; and b. contacting at a first pH the EV associated with the first binding partner with a second binding partner, wherein the second binding partner comprises the Fc binding site of the FcRn and is associated with a solid matrix; and c. eluting the EV associated with the first binding partner from the solid matrix at a second pH. In certain embodiments, the method comprises a washing step at the first pH. In certain embodiments, the first pH is below 6.5. In certain embodiments, the second pH is above 7.4.

In another aspect, provided herein is a method for purifying an extracellular vesicle (EV), wherein said method comprises: a. providing the EV wherein the EV is associated with a first binding partner, wherein the first binding partner is capable of binding to the Fc binding site of an FcRn in a pH dependent manner and comprises or consists of a polypeptide described herein; and b. contacting at a first pH the EV associated with the first binding partner with a second binding partner, wherein the second binding partner comprises the Fc binding site of the FcRn and is associated with a solid matrix; and c. eluting the EV associated with the first binding partner from the solid matrix at a second pH. In certain embodiments, the method comprises a washing step at the first pH. In certain embodiments, the first pH is below 6.5. In certain embodiments, the second pH is above 7.4.

The present disclosure aims to satisfy existing needs within the art, for instance, to provide means for the isolation/separation of extracellular vesicles that can be effected at conditions (e.g., pH values) closer to physiological conditions, and to enable longer half-life of EVs in the circulation to considerably enhance the therapeutic potential of EVs for therapeutic delivery.

Provided herein are FcRn binding polypeptides comprising transmembrane domains (herein often referred to as FcRn binders).

In one aspect, provided herein is a system for purification of an nanovesicle of interest (e.g., an EV), wherein the system comprises a neonatal Fc Receptor (FcRn) Binder and a mammalian FcRn, wherein the FcRn Binder and FcRn bind to each other with high affinity under a first set of conditions and with low affinity under a second set of conditions.

### 3.1 Illustrative Embodiments

1. A polypeptide, wherein the polypeptide comprises:
   a. a transmembrane domain; and
   b. a modified Fc domain of an immunoglobulin that
      i. is capable of specifically binding to the Fc binding site of an FcRn; and
      ii. lacks the ability to form homodimers.
2. The polypeptide of paragraph 1, wherein the equilibrium dissociation constant of the modified Fc domain bound to FcRn at a pH of 6.5 has a value of at most 10⁻⁴M.
3. The polypeptide of paragraph 1 or 2, wherein the equilibrium dissociation constant of the modified Fc domain bound to FcRn at a pH of 7.4 has a value of at least 10⁻⁴ M.
4. The polypeptide of any one of paragraphs 1-3, wherein the modified Fc domain is capable of specifically binding to the amino acid sequence between position 135-158 of human FcRn (SEQ ID NO: 7) and/or mouse FcRn (SEQ ID NO: 8).
5. The polypeptide of any one of paragraphs 1-4, wherein the modified Fc domain is capable of specifically binding to the amino acid sequence LNGEEFMX₁FX₂X₃X₄X₅GX₆WX₇GX₈W (SEQ ID NO:6), wherein X₁, X₂, X₃, X₄, X₅, X₆, X₇ and X₈ each is any amino acid.
6. The polypeptide of any one of paragraphs 1-5, wherein said polypeptide does not substantially bind to C1q, FcγRI, FcγRII or FcγRIII.
7. The polypeptide of any one of paragraphs 1-6, wherein:
   a. the complement dependent cytotoxicity (CDC) activity of the modified Fc domain;
   b. the antibody dependent cell mediated cytotoxicity (ADCC) activity of the modified Fc domain;
   c. the antibody dependent cell mediated phagocytosis (ADCP) activity of the modified Fc domain; and/or
   d. the antibody dependent intracellular neutralization (ADIN) activity of the modified Fc domain
   is decreased by at least 10%, 20%, 30%, 40%, or 50% compared to the unmodified Fc domain.
8. The polypeptide of any one of paragraphs 1-7, wherein:
   a. the complement dependent cytotoxicity (CDC) activity of the modified Fc domain;
   b. the antibody dependent cell mediated cytotoxicity (ADCC) activity of the modified Fc domain;
   c. the antibody dependent cell mediated phagocytosis (ADCP) activity of the modified Fc domain; and/or
   d. the antibody dependent intracellular neutralization (ADIN) activity of the modified Fc domain
   is decreased by at least 1.5, 2, 3, 4, or 5-fold, compared to the unmodified Fc domain.
9. The polypeptide of any one of paragraphs 1-8, wherein the FcRn binding polypeptide comprises from N-terminus to C-terminus:
   a. a modified CH2 domain that is modified relative to the unmodified CH2 domain to decrease effector function;
   b. a modified CH3 domain that is modified relative to the unmodified CH3 domain to lack the homodimerize;
   c. a linker sequence; and
   d. a transmembrane domain.
10. The polypeptide of any one of paragraphs 1-9, wherein the FcRn binding polypeptide comprises from C-terminus to N-terminus:
   a. a modified CH3 domain that is modified relative to the unmodified CH3 domain to lack the homodimerize;
   b. a modified CH2 domain that is modified relative to the unmodified CH2 domain to decrease effector function;
   c. a linker sequence; and
   d. a transmembrane domain.
11. The polypeptide of any one of paragraphs 1-10, wherein the transmembrane domain is a multipass transmembrane domain.
12. The polypeptide of any one of the paragraphs 1-11, further comprising a targeting domain selected from the group consisting of: scFv, (scFv)₂, Fab, Fab′, F(ab′)₂, F(abl)₂, Fv, dAb, Fd fragments, diabodies, F(ab)2, F(ab′), F(ab′)3, Fd, Fv, disulfide linked Fv, dAb, sdAb, nanobody, CDR, di-scFv, bi-scFv, tascFv (tandem scFv), AVIBODY (e.g., diabody, triabody, tetrabody), T-cell engager (BiTE), V-NAR domain, Fcab, IgGACH2, DVD-Ig, probody, intrabody, DARPin, Centyrin, affibody, affilin, affitin, anticalin, avimer, Fynomer, Kunitz domain peptide, monobody, adnectin, tribody, and nanofitin.
13. A nucleic acid encoding the polypeptide of any one of paragraphs 1-12.
14. An expression vector comprising the nucleic acid of paragraph 13.
15. A cell comprising the nucleic acid of paragraph 13 or the expression vector of paragraph 14.
16. An extracellular vesicle comprising the polypeptide of any one of paragraphs 1 to 12.
17. A hybridosome comprising the polypeptide of any one of paragraphs 1 to 12.
18. A method for purifying an extracellular vesicle (EV), wherein said method comprises:
   a. providing the EV wherein the EV is associated with a first binding partner, wherein the first binding partner is capable of binding to the Fc binding site of an FcRn in a pH dependent manner; and
   b. contacting at a first pH the EV associated with the first binding partner with a second binding partner, wherein the second binding partner comprises the Fc binding site of the FcRn and is associated with a solid matrix; and
   c. eluting the EV associated with the first binding partner from the solid matrix at a second pH.
19. The method of paragraph 18, wherein the method comprises a washing step at the first pH.
20. The method of paragraph 18 or 19, wherein the first pH is below 6.5.
21. The method of any one of paragraphs 18 to 20, wherein the second pH is above 7.4.
22. A method for purifying an EV, wherein said method comprises:
   a. providing the EV wherein the EV is associated with a first binding partner, wherein the first binding partner is capable of binding to the Fc binding site of an FcRn in a pH dependent manner and comprises or consists of the polypeptide of any one of paragraphs 1-12; and
   b. contacting at a first pH the EV associated with the first binding partner with a second binding partner, wherein the second binding partner comprises the Fc binding site of the FcRn and is associated with a solid matrix; and
   c. eluting the EV associated with the first binding partner from the solid matrix at a second pH.
23. The method of paragraph 22, wherein the method comprises a washing step at the first pH.
24. The method of paragraph 22 or 23, wherein the first pH is below 6.5.
25. The method of any one of paragraphs 22 to 24, wherein the second pH is above 7.4.

### 4. BRIEF DESCRIPTION OF FIGURES

FIG. 1 is a schematic of a nanovesicle comprising an FcRn binding polypeptide that contains a type 1 transmembrane domain.

FIG. 2 depicts examples of the location of the modified Fc (CH2 and monomeric CH3) in relation to the transmembrane helix (TMH) of different transmembrane scaffolds, including T1 scaffolds, T2 scaffolds, and PT scaffolds. For PT scaffolds, the FcRn binding site can be located at the N-terminus (PTa), C-terminus (PTb) or in the extracellular loops (PTc).

FIG. 3. Exemplary structures of an FcRn binding polypeptide, comprising a monomeric Fc fused to a scaffold protein derived from the extracellular domain of Eph receptors.

FIG. 4. Western blot showing engineered EVs purified from the conditioned media.

FIG. 5A-5D. Flow cytometry histograms of different cell lines stained with a fluorescent anti-human Fc domain antibody as described in Example 2.

FIG. 6. Nanoparticle tracking analysis (NTA) measurement of EVs incubated at different pHs for 20 minutes.

FIG. 7. Anti-FcRn western blot showing the purification of scFcRn

FIG. 8. Anti-EphA4 western blot showing the detection of EphA4 fusion proteins expressed from constructs in concentrated conditioned media, which were loaded onto a scFcRn column. The first lane is the load, the second lane is a sample of the flow through and the third lane is a sample of the eluted fraction.

FIG. 9A and FIG. 9B. Anti-EphA4 western blot showing the detection of EphA4 fusion proteins expressed from constructs in concentrated conditioned media, which were loaded onto a scFcRn column at different pHs. In FIG. 9A the conditioned media was not acidified while in FIG. 9B the conditioned media was acidified as described in example 7. The first lane is the elution sample, the second lane is a sample of the flow through and the third lane is a sample of the conditioned media.

FIG. 10A and FIG. 10B depict binding curves from an human FcRn binding immunoassay with EVs expressing the modified Fc domain (FIG. 10A), native EVs (FIG. 10A), human IgG1 (FIG. 10B) and mouse IgG1 (FIG. 10B).

FIG. 11. DNA vector copy number per ul of mouse plasma on days 3, 6, 21 and 24 after IV administration of EVs comprising a scaffold protein displaying a modified Fc domain vs a LNP formulation.

### 5. DETAILED DESCRIPTION

Provided herein are polypeptides comprising a transmembrane domain and an FcRn binding site (e.g., a modified Fc domain of an immunoglobulin). In certain embodiment, the FcRn binding site (e.g., modified Fc domain of an immunoglobulin) is capable of specifically binding to the Fc binding site of an FcRn and lacks the ability to form homodimers. Various aspects and embodiments of the polypeptides are described in Section 5.2.

Also provided are nucleic acids encoding a polypeptide described herein, expression vectors comprising a nucleic acid described herein, and cells comprising a nucleic acid or expression vector described herein, all of which are further described in Section 5.3.

Further provided are nanovesicles (e.g., EVs and hybridosomes) comprising a polypeptide described herein. Nanovesicles (e.g., EVs and hybridosomes) are further described in Section 5.4.

Methods of producing a nanovesicle (e.g., an EV or hybridosome) are provided and are further described in Section 5.4. Methods of purifying a nanovesicle (e.g., an EV or hybridosome) are also provided and are further described in Section 5.5.

Compositions and kits comprising a polypeptide, a nanovesicle (e.g., an EV or hybridosome), a nucleic acid, an expression vector, or a cell described herein are provided and further described in Section 5.6.

Therapeutic and diagnostic uses of a polypeptide, a nanovesicle (e.g., an EV or hybridosome), a composition, or a kit described herein are provided and further described in Section 5.7.

It is an object of the present disclosure to overcome problems associated with the isolation and purification of nanovesicles (e.g. EVs). Furthermore, the present disclosure aims to satisfy other existing needs within the art, for instance, to develop generally applicable affinity purification strategies for purifying nanovesicles (e.g. EV) at high yields and with high specificity. In particular, the previously known methods for purifying exosomes are not ideally suited to large scale production and scale up that would be necessary for commercial production of EV therapeutics. The present disclosure allows much larger scale purification of engineered EVs with high affinity than would be achievable with previously known methods.

We have developed a method and compositions that enable specific interaction between engineered nanovesicles (e.g. EVs) and the FcRn receptor that can be present in cells or as a binding agent for affinity chromatography. In particular, it was demonstrated that some nanovesicles (e.g. EVs) have low colloidal stability at low pH, contain pH-labile components and do not lend themselves to low pH elution usually employed in fc-binding based affinity chromatography but efficient affinity chromatography requires a fc-binding agent whose binding specificity can be modulated in a pH range of about 5-8.

Disclosed herein are nanovesicle (e.g. EVs) that comprise FcRn binders that bind to FcRn with high affinity and specificity. Advantageously, several of the FcRn binders described herein have one or more improved or desired pharmacokinetic properties, such as circulating half-life. Without wishing to be bound theory, it is believed that nanovesicles (e.g. EVs) can have a range of circulating half-lives in humans, and circulating half-life can affect, e.g., interaction with serum and cell components, interaction with FcRn, receptor mediated endocytosis, drug doses, and generation of anti-drug antibodies. Nucleic acid molecules encoding the fusion polypeptides, expression vectors, host cells, compositions (e.g., pharmaceutical compositions), kits, containers, and methods for making the FcRn binding nanovesicles (e.g. EVs), are also provided. The polypeptides (e.g., antibody molecules or fusion proteins) and pharmaceutical compositions disclosed herein can be used (alone or in combination with other agents or therapeutic modalities) to treat, prevent, and/or diagnose disorders and conditions, e.g., disorders and conditions associated with a target molecule (e.g., protein) or cell, e.g., a disorder or condition described herein.

Without wishing to be bound by theory, it is believed that in some embodiment, the engineering of Fc for FcRn binding or half-life extension as disclosed herein is performed in the context of the various effector functions mediated by Fc. For example, structural information can be used to interrogate the interaction of Fc with FcRn at neutral and acidic pH. Using this structural information, different structures for improving FcRn binding at acidic pH can be identified. Fc mutations can be combined and assessed for binding to FcRn and other Fc receptors. For example, Fc variants that confer enhancement in half-life and retain and in some cases have decreased effector functions such as ADCC and CDC can be identified. With the increasing interest to employ nanovesicles (e.g. EVs) as therapeutics for prevention and treatment of different diseases, there have been greater needs to develop nanovesicles comprising FcRn binding polypeptides with long half-life, e.g., to treat or prevent chronic diseases.

Furthermore, all FcRn binding polypeptides and proteins identified herein can be freely combined in fusion proteins using conventional strategies for fusing polypeptides. As a non-limiting example, all FcRn binding polypeptides described herein may be freely combined in any combination with one or more EV polypeptides. Also, FcRn binding polypeptides may be combined with each other to generate fusion proteins comprising more than one FcRn binding polypeptide. Moreover, any and all features (for instance any and all members of a Markush group described herein) can be freely combined with any and all other features (for instance any and all members of any other Markush group described herein), e.g. any EV comprising an FcRn binding polypeptide may be purified and/or isolated using any FcRn domain containing polypeptides. Furthermore, when teachings herein refer to nanovesicles (e.g. EVs) (and/or the EVs comprising FcRn binding polypeptides) in singular and/or to nanovesicles (e.g. EVs) as discrete natural nanoparticle-like vesicles it should be understood that all such teachings are equally relevant for and applicable to a plurality of nanovesicles (e.g. EVs) and populations of nanovesicles (e.g. EVs).

### 5.1 Definitions

As used herein, the singular forms “a,” “an,” and “the” include plural referents unless the content clearly dictates otherwise. Thus, for example, reference to “a polypeptide” may include two or more such molecules, and the like.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

The terms “genetically modified” and “genetically engineered” EV indicate that the EV is derived from a genetically modified/engineered cell usually comprising a recombinant or exogenous protein product which is incorporated into the nanovesicles (e.g. EVs) produced by those cells. The term “modified EV” indicates that the vesicle has been modified either using genetic or chemical approaches, for instance via genetic engineering of the EV-producing cell or via e.g. chemical conjugation, for instance to attach moieties to the exosome surface.

A “binding domain” is a peptide region, such as a fragment of a polypeptide derived from an immunoglobulin (e.g., an antibody), that specifically binds one or more specific binding partners. If a plurality of binding partners exists, those partners share binding determinants sufficient to detectably bind to the binding domain. Preferably, the binding domain is a contiguous sequence of amino acids.

The term “FcRn” denotes the neonatal Fc-receptor. FcRn functions to salvage IgG from the lysosomal degradation pathway, resulting in reduced clearance and long half-life. The FcRn is a heterodimeric protein consisting of two polypeptides: a 50 kDa class I major histocompatibility complex-like protein (a-FcRn) and a 15 kDa β2-microglobulin (β2ιη). FcRn binds with high affinity to the CH2-CH3 portion of the Fe domain of IgG. FcRn interacts with the Fc region of antibodies to promote recycling through rescue from normal lysosomal degradation. This process is a pH-dependent process that occurs in the endosomes at acidic pH (e.g., a pH less than 6.5) but not under the physiological pH conditions of the bloodstream (e.g., a non-acidic pH). An acidic pH is a pH less than about 7.0, e.g., about pH 6.5, at about pH 6.0, at about pH 5.5, at about pH 5.0. An elevated, non-acidic pH is a pH of about 7 or greater, such as about pH 7.4, about pH 7.6, about pH 7.8, about pH 8.0, about pH 8.5, or about pH 9.0. FcRn then facilitates the recycling of FcRn binding polypeptides to the cell surface and subsequent release into the blood stream upon exposure of the FcRn-FcRn binding polypeptides complex to the neutral pH environment outside the cell.

As used herein, an “FcRn binding site” refers to the region of an Fc polypeptide that binds to FcRn.

As used herein, an “Fc binding site” refers to the region of an FcRn polypeptide that binds to Fc domain of an immunoglobulin.

The term “specifically binds” refers to a molecule (e.g., a Fab, an scFv, or a modified Fc polypeptide (or a target-binding portion thereof) that binds to an epitope or target with greater affinity, greater avidity, and/or greater duration to that epitope or target in a sample than it binds to another epitope or non-target compound (e.g., a structurally different antigen). In some embodiments, a Fab, scFv, or modified Fc polypeptide (or a target-binding portion thereof) that specifically binds to an epitope or target is a Fab, scFv, or modified Fc polypeptide (or a target-binding portion thereof) that binds to the epitope or target with at least 5-fold greater affinity than other epitopes or non-target compounds, e.g., at least 6-fold, 7-fold, 8-fold, 9-fold, 10-fold, 25-fold, 50-fold, 100-fold, 1000-fold, 10,000-fold, or greater affinity. The term “specific binding,”, “specifically binds to”, or “is specific for” a particular epitope or target, as used herein, can be exhibited, for example, by a molecule having an equilibrium dissociation constant KD for the epitope or target to which it binds of, e.g, 10⁻⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. It will be recognized by one of skill that a Fab or scFv that specifically binds to a target from one species may also specifically bind to orthologs of that target.

The terms “CH3 domain” and “CH2 domain” as used herein refer to immunoglobulin constant region domain polypeptides. For purposes of this application, a CH2 and CH3 domain polypeptide may be numbered by the IMGT (ImMunoGeneTics) numbering scheme, in which the CH2 domain numbering is 1-110 and the CH3 domain numbering is 1-107, according to the IMGT Scientific chart numbering (IMGT website). CH2 and CH3 domains are part of the Fc region of an immunoglobulin. Alternatively, a CH2 and CH3 domain polypeptide may be numbered by the EU numbering scheme, in which the CH2 domain numbering spans residues 231-340 and the CH3 domain numbering spans residues 341-447, according to the EU numbering scheme. An Fc region refers to the segment of amino acids from about position 231 to about position 447 as numbered according to the EU numbering scheme. The “EU numbering scheme” refers to the EU numbering convention for the constant regions of an antibody, as described in Kabat et al, Sequences of Proteins of Immunological Interest, U.S. Dept. Health and Human Services, 5th edition, 1991, each of which is herein incorporated by reference in its entirety.

As used herein, the term “scaffold protein” refers to a polypeptide that can be used to anchor a FcRn binding polypeptide to the nanovesicle. In some aspects, the scaffold protein is a polypeptide that does not naturally exist in a nanovesicle (e.g. an EV). In some embodiments, the scaffold protein comprises a synthetic polypeptide. In some embodiments, the scaffold protein comprises a modified protein, wherein the corresponding unmodified protein naturally exists in the nanovesicle, e.g., the exosome. In some embodiments, the scaffold protein comprises a protein that naturally exists in the EV, or a fragment thereof, e.g., a fragment of an EV protein, where the protein is expressed at a higher level than the naturally occurring level.

In some embodiments, the scaffold protein comprises a fusion protein, comprising (i) a naturally occurring EV protein or a fragment thereof and (ii) a heterologous polypeptide (e.g., FcRn binding polypeptide, an antigen binding domain, or any combination thereof).

As used herein, the term “scaffold protein” of the present disclosure, or grammatical variants, can be:
(i) a polypeptide (naturally expressed, chemically or enzymatically synthesized, or produced recombinantly) that comprises at least one FcRn binding site and further comprises a transmembrane domain that spans the membrane of nanovesicles, e.g., exosomes;
(ii) any functional fragment of (i);
(iii) any functional variant of (i) or (ii);
(iv) any derivative of any of (i)-(iii);
(v) any peptide corresponding to a domain or combination thereof derived from a protein in (i) that can span the membrane of nanovesicles, (e.g., exosomes), or a molecule comprising such polypeptide;
(vi) a FcRn binding polypeptide described herein;
(vii) a molecule of any of (i) to (vi) comprising at least one non-natural amino acid; or
(viii) any combination of (i)-(vii);
which is suitable for use as a scaffold to target (attach) a FcRn binding site to the surface nanovesicles, e.g., exosomes.

The term “surface decorated” as used herein refers to nanovesicles comprising a scaffold protein to which a molecule of interest (e.g., a protein), is attached. The scaffold protein can be changed by a chemical, a physical, or a biological method or by being produced from a cell being modified by a chemical, a physical, or a biological method. Specifically, the scaffold protein can be changed via genetic engineering so that a cell previously modified by genetic engineering produces such modified scaffold proteins.

“Fused” polypeptide sequences are connected via a peptide bond between two subject polypeptide sequences.

As used herein, the term “domain” refers to a unit (e.g., segment) of a polypeptide that can independently fold into a stable tertiary structure). Generally, domains are responsible for discrete functional properties of proteins, and in many cases may be added, removed, or transferred to other proteins without loss of function of the remainder of the protein and/or of the domain. Several distinct domains can be joined together in different combinations, forming multi-domain polypeptides. Traditionally, the length of polypeptides spanning domains have been elucidated by the use of atomic coordinates from experimentally determined three-dimensional structures of proteins. More recently, proteins lacking experimentally determined three-dimensional (3D) structures have been assigned domains by computational methods based on sequence homology. Since a large number of proteins do not have resolved structures, sequence-based approaches have been gaining much more attention. The sequence-based approaches include template-based, homologous-modeling-based and machine-learning-based techniques, depending on whether the prediction methods make use of 3D structure or homologous sequences as reviewed in Wang, Yan et al. Computational and structural biotechnology journal vol. 19 1145-1153. 2 Feb. 2021. Several computationally predicted domains are cataloged in publicly available databases (e.g., Pfam database as described in Pfam: The protein families database in 2021: J. Mistry, S. et al, Nucleic Acids Research (2020) or the NCBI Conserved Domain Database (CDD) https://www.ncbi.nlm.nih.gov/Structure/cdd/cdd.shtml).

The term “inter-domain linkers” refers to the segment of a polypeptide that ties two neighboring domains together. Inter-domain linkers provide flexibility to facilitate domain motions and to regulate the inter-domain geometry as described in Bhaskara R M, et al., J Biomol Struct Dyn. 2013 December; 31(12):1467-80. The inter-domain linkers modulate the interactions of adjacent domains by their lengths, conformations, intermolecular interactions, and local structure, thereby affecting the overall inter-domain geometry. Above mentioned databases based on predicted structural domains (Pfam database or NCBI Conserved Domain Database) provide generalizations of domains and may offer only an approximation of a domain boundary (e.g., to distinguish between residues that are within a domain or are inter-domain linkers). Hence, the domain sequences described herein (e.g., sequences in Tables 2-20) may include polypeptide sequences that comprise corresponding domain as well as inter-domain linkers. In some embodiments the 1, 2, 3, 4, 5, 6, 7, 8, 9 or 10 amino acid residues at the N- or C terminal of the cataloged domain sequences can be inter-domain linkers. Those skilled in the art may determine the segments of a polypeptide chain corresponding to domains and inter-domain linkers, and where a transition from a domain (i.e., at a domain boundary) to the inter-domain linker occurs.

The term “fusion polypeptide” refers to a FcRn binding polypeptide or an amino acid sequence derived from a polypeptide operably linked to at least a second polypeptide or an amino acid sequence derived from at least a second polypeptide. The individualized elements of the fusion protein can be linked in any of a variety of ways, including for example, direct attachment, the use of an intermediate or a spacer peptide, the use of a linker region, the use of a hinge region or the use of both a linker and a hinge region. In some embodiments, the linker region may fall within the sequence of the hinge region, or alternatively, the hinge region may fall within the sequence of the linker region. Preferably, the linker region is a peptide sequence. For example, the linker peptide includes anywhere from zero to 40 amino acids, e.g., from zero to 35 amino acids, from zero to 30 amino acids, from zero to 25 amino acids, or from zero to 20 amino acids. Preferably, the hinge region is a peptide sequence. For example, the hinge peptide includes anywhere from zero to 75 amino acids, e.g., from zero to 70 amino acids, from zero to 65 amino acids or from zero to 62 amino acids.

The terms “wild-type”, “native”, and “naturally occurring” with respect to a CH3 or CH2 domain are used herein to refer to a domain that has a sequence that occurs in nature.

As used herein, the term “mutant” with respect to a mutant polypeptide or mutant polynucleotide is used interchangeably with “variant”. In particular embodiments, a variant with respect to a given wild-type CH3 or CH2 domain of IgG (e.g. the Fc domain) reference sequence or a wild type scaffold protein reference sequence can include naturally occurring allelic variants. A “non-naturally” occurring variant refers to a variant or mutant domain that is not present in a cell in nature and that is produced by genetic modification, e.g., using genetic engineering technology or mutagenesis techniques, of a parental Fc domain polynucleotide introducing appropriate modifications into the nucleic acid sequence encoding the polypeptide, or by protein/peptide synthesis. A “variant” includes any sequence comprising at least one amino acid mutation with respect to wild-type. Mutations may include substitutions, insertions, and deletions (e.g., truncation) of one or more amino acids as well as frameshift or rearrangement in another protein. Similarly, the term “variant,” with respect to a polynucleotide, refers to a polynucleotide that differs in nucleotide sequence from a specified parental polynucleotide. The identity of the parental polypeptide or polynucleotide will be apparent from context. A variant can include one or more specific substitutions, insertions, and/or deletions as well as having a % sequence identity to the parental sequence.

The term “amino acid substitution” denotes the replacement of at least one existing amino acid residue with another different amino acid residue (replacing amino acid residue). The replacing amino acid residue may be a “naturally occurring amino acid residues” and selected from the group consisting of alanine (three letter code: ala, one letter code: A), arginine (arg, R), asparagine (asn, N), aspartic acid (asp, D), cysteine (cys, C), glutamine (gin, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

The term “amino acid insertion” denotes the incorporation of at least one amino acid residue at a predetermined position in an amino acid sequence. In one embodiment the insertion will be the insertion of one or two amino acid residues. The inserted amino acid residue(s) can be any naturally occurring or non-naturally occurring amino acid residue. The term “amino acid deletion” denotes the removal of at least one amino acid residue at a predetermined position in an amino acid sequence.

The term “non-naturally occurring amino acid residue” denotes an amino acid residue, other than the naturally occurring amino acid residues as listed above, which can be covalently bound to the adjacent amino acid residues in a polypeptide chain. Examples of non-naturally occurring amino acid residues are norleucine, ornithine, norvaline, homoserine. Further examples are listed in Ellman, et al., Meth. Enzym. 202 (1991) 301-336. Exemplary method for the synthesis of non-naturally occurring amino acid residues are reported in, e. g., Noren, et al., Science 244 (1989) 182 and Ellman et al., supra.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The terms “extracellular vesicle”, “EV” or “exosome” are used interchangeably herein and shall be understood to relate to any type of vesicle that is obtainable from a cell in any form, for instance a microvesicle (e.g. any vesicle shed from the plasma membrane of a cell), an exosome (e.g. any vesicle derived from the endosomal, lysosomal and/or endo-lysosomal pathway), an apoptotic body, ARMMs (arrestin domain containing protein 1-mediated microvesicles), fusosomes, a microparticle and cell derived vesicular structures. Generally extracellular vesicles range in hydrodynamic diameter from 20 nm to 1000 nm and can comprise various macromolecular cargo either within the internal space, displayed on the external surface of the extracellular vesicle, and/or spanning the membrane. Said cargo can comprise nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion, sonication or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells. In a preferred embodiment, the EVs as per the present disclosure are exosomes, microvesicles (MVs), or any other type of vesicle which is secreted from the endosomal, endolysomal and/or lysosomal pathway or from the plasma membrane of a parental cell. Furthermore, when teachings herein refer to EVs in singular and/or to EVs as discrete natural nanoparticle-like vesicles it should be understood that all such teachings are equally relevant for and applicable to a plurality of EVs and populations of EVs.

It will be clear to the skilled artisan that when describing medical and scientific uses and applications of the nanovesicles (e.g. EVs), the present disclosure normally relates to a plurality of nanovesicles (e.g. EVs), i.e. a population of nanovesicles (e.g. EVs) which may comprise thousands, millions, billions or even trillions of nanovesicles (e.g. EVs). As can be seen from the experimental section below, nanovesicles (e.g., EVs) may be present in concentrations such as 10^{5,} 10⁸, 10¹⁰, 10¹¹, 10¹², 10¹³, 10¹⁴, 10¹⁵, 10¹⁸, 10²⁵, 10³⁰ nanovesicles (often termed “particles”) per unit of volume (for instance per ml), or any other number larger, smaller or anywhere in between. Individual nanovesicles (e.g. EVs) when present in a plurality constitute an EV population. Thus, naturally, the present disclosure pertains both to individual nanovesicles (e.g. EVs) and populations comprising nanovesicles (e.g. EVs), as will be clear to the skilled person.

The term “nanovesicles” refers to lipid nanovesicles derived from a source cell (i.e. extracellular vesicles), or synthetic lipid nanoparticle, and natural/synthetic hybrids (such as a hybridosome). A nanovesicle typically comprises lipids or fatty acids as well as polypeptides, and may further comprise a payload, a targeting moiety or other molecules. Furthermore, when teachings herein refer to a nanovesicle in singular it should be understood that all such teachings are equally relevant for and applicable to a plurality of nanovesicles and populations of nanovesicles. It will be clear to the skilled person that when describing medical and scientific uses and applications of the nanovesicles, the present disclosure normally relates to a plurality of nanovesicles, i.e. a population of nanovesicles which may comprise thousands, millions, billions or even trillions of nanovesicles. As can be seen from the experimental section below, nanovesicles may be present in concentrations such as 10⁵, 10⁸, 10¹⁰, 10¹¹, 10¹², 10¹³, 10¹⁴, 10¹⁵, 10¹⁸, 10²¹, 10³⁰ particles per unit of volume (for instance per ml), or any other number larger, smaller or anywhere in between. Individual nanovesicles when present in a plurality constitute a nanovesicle population. Thus, naturally, the present disclosure pertains both to individual nanovesicles and populations comprising nanovesicles.

Furthermore, the nanovesicles (e.g. EVs) of the present disclosure may also comprise additional payloads, in addition to the FcRn binding polypeptide which may be bound to the nanovesicle surface.

The terms “source cell” or “EV source cell” or “parental cell” or “cell source” or “EV-producing cell” or any other similar terminology may be understood to relate to any type of mammalian cell that is capable of producing nanovesicles (e.g. EVs) under suitable conditions, for instance in suspension culture or in adherent culture or any in other type of culturing system. Source cells as per the present disclosure may also include cells producing nanovesicles (e.g. EVs) in vivo. The source cells per the present disclosure may be selected from a wide range of cells and cell lines which may grow in suspension or adherent culture or being adapted to suspension growth. Generally, nanovesicles (e.g. EVs) may be derived from essentially any cell source, be it a primary cell source or an immortalized cell line. The EV source cells may be any embryonic, fetal, and adult somatic stem cell types, including induced pluripotent stem cells (iPSCs) and other stem cells derived by any method. The source cell may be either allogeneic, autologous, or even xenogeneic in nature to the patient to be treated, i.e. the cells may be from the patient himself or from an unrelated, matched or unmatched donor. In certain contexts, allogeneic cells may be preferable from a medical standpoint, as they could provide immuno-modulatory effects that may not be obtainable from autologous cells of a patient suffering from a certain indication. For instance, in the context of treating inflammatory or degenerative diseases, allogeneic MSCs or AEs may be highly beneficial as EV-producing cell sources due to the inherent immuno-modulatory of their nanovesicles (e.g. EVs) and in particular their nanovesicles (e.g. EVs). Cell lines of particular interest include human umbilical cord endothelial cells (HUVECs), human embryonic kidney (HEK) cells such as HEK293 cells, HEK293T cells, serum free HEK293 cells, suspension HEK293 cells, endothelial cell lines such as microvascular or lymphatic endothelial cells, erythrocytes, erythroid progenitors, chondrocytes, MSCs of different origin, amnion cells, amnion epithelial (AE) cells, any cells obtained through amniocentesis or from the placenta, airway or alveolar epithelial cells, fibroblasts, endothelial cells, epithelial cells, etc.

The term “buffer substance” denotes a substance that when in solution can level changes of the pH value of the solution e.g. due to the addition or release of acidic or basic substances.

As used herein, the terms “isolate”, “isolated”, and “isolating” or “purify”, “purified”, and “purifying” as well as “extracted” and “extracting” are used interchangeably and refer to the state of a preparation (e.g., a plurality of known or unknown amount and/or concentration) of desired FcRn binding nanovesicles (e.g. EVs), that have undergone one or more processes of purification, e.g., a selection or an enrichment of the desired FcRn binding EV preparation. In some embodiments, isolating or purifying as used herein is the process of removing, partially removing (e.g., a fraction) of the nanovesicles comprising FcRn binding polypeptides from a sample containing producer cells. In some embodiments, an isolated nanovesicles comprising FcRn binding polypeptides composition has no detectable undesired activity or, alternatively, the level or amount of the undesired activity is at or below an acceptable level or amount. In other embodiments, an isolated exosome composition has an amount and/or concentration of desired nanovesicles comprising FcRn binding polypeptides at or above an acceptable amount and/or concentration. In other embodiments, the isolated nanovesicles comprising FcRn binding polypeptides composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%), or greater than 99.99990%) as compared to the starting material. In some embodiments, isolated nanovesicles comprising FcRn binding polypeptides preparations are substantially free of residual biological products. In some embodiments, the isolated Nanovesicles comprising FcRn binding polypeptides preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the Nanovesicles comprising FcRn binding polypeptides composition contains no detectable producer cells and that only Nanovesicles comprising FcRn binding polypeptides are detectable.

The terms “polynucleotide” and “nucleic acid” interchangeably refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and

As is normally the case with fusion proteins, the two components that are normally included in the fusion protein (i.e. FcRn binding polypeptide and scaffold protein comprising the transmembrane domain) may be linked directly in a contiguous fashion in the fusion protein, or they may be linked and/or attached to each other using a variety of linkers. Any of the peptide linkers may comprise a length of at least 5 residues, at least 10 residues, at least 15 residues, at least 20 residues, at least 25 residues, at least 30 residues or more. In other embodiments, the linkers comprise a length of between 2-4 residues, between 2-4 residues, between 2-6 residues, between 2-8 residues, between 2-10 residues, between 2-12 residues, between 2-14 residues, between 2-16 residues, between 2-18 residues, between 2-20 residues, between 2-22 residues, between 2-24 residues, between 2-26 residues, between 2-28 residues, or between 2-30 residues. n some embodiments, the first linker comprises a flexible linker. In some embodiments, the first linker comprises a glycine-serine linker, i.e., a linker that consists primarily of, or entirely of, stretches of glycine and serine residues. In some embodiments, the first linker comprises a (G₄S)n linker (GGGGS)n (SEQ ID NO: 1), in which “n” indicates the number of repeats of the motif and is an integer number from 1 to 10. In some embodiments, the first linker comprises a G₄S (GGGGS; SEQ ID NO: 2) linker, a (G₄S)₂ (GGGGS GGGGS; SEQ ID NO:3) linker, a (G₄S)₃ (GGGGS GGGGS GGGGS; SEQ ID NO: 4) linker, or a (G₄S)₂-G₄ (SEQ ID NO: 5) linker.

By “single-chain Fv” or “scFv” as used herein are meant antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding. Methods for producing scFvs are well known in the art. For a review of methods for producing scFvs see Pluckthun in The Pharmacology of Monoclonal Antibodies, vol. 1 13, Rosenburg and Moore eds. Springer-Verlag, New York, pp. 269-315 (1994).

It is understood that wherever aspects or embodiments are described herein with the language “comprising,” otherwise analogous aspects described in terms of “consisting of” and/or “consisting essentially of” are also provided.

### 5.2 Polypeptides of the Disclosure

In particular, provided herein are certain FcRn binding polypeptides comprising (i) at least one FcRn binding site and (ii) a transmembrane (TM) domain. In certain embodiments, the transmembrane domain is a multipass transmembrane domain.

In one aspect, FcRn binding polypeptide for use with the methods and compositions provided herein shall be understood to relate to a polypeptide which comprise a FcRn binding site that can bind the FcRn with a high affinity at a pH below physiological pH and is anchored to a membrane by at least one transmembrane domain or fragments thereof.

In certain embodiments, the FcRn binding polypeptide comprises a transmembrane domain (e.g. scaffold protein) and a FcRn binding site (e.g., a modified Fc domain of an immunoglobulin) that is capable of specifically binding to the Fc binding site of a neonatal Fc receptor, and lacks the ability to form homodimers. In certain embodiments, the equilibrium dissociation constant of the FcRn binding site (e.g., modified Fc domain of an immunoglobulin) bound to the FcRn at a pH of 6.5 has a value of at most 10⁻⁴ M. In certain embodiments, the equilibrium dissociation constant of the FcRn binding site (e.g., modified Fc domain of an immunoglobulin) bound to the FcRn at a pH of 7.4 has a value of at least 10⁻⁴ M. In certain embodiments, the FcRn binding site (e.g., modified Fe domain of an immunoglobulin) is capable of specifically binding to the amino acid sequence

SEQ ID NO: 6
LNGEEFMX₁FX₂X₃X₄X₅GX₆WX₇GX₈W,

wherein X₁, X₂, X₃, X₄, X₅, X₆, X₇ and X₈ each is any amino acid. In certain embodiments, the FcRn binding site (e.g., modified Fc domain of an immunoglobulin) is capable of specifically binding to the amino acid sequence between position 135-158 of human FcRn (SEQ ID NO:7) and/or mouse FcRn (SEQ ID NO:8).

| Seq ID | Name | |
|---|---|---|
| 7 | Human | MGVPRPQPWALGLLLFLLPGSLGAESHLSLLYH |
| | FcRn | LTAVSSPAPGTPAFWVSGWLGPQQYLSYNSLRG |
| | | EAEPCGAWVWENQVSWYWEKETTDLRIKEKLF |
| | | LEAFKALGGKGPYTLQGLLGCELGPDNTSVPTA |
| | | KFALNGEEFMNFDLKQGTWGGDWPEALAISQR |
| | | WQQQDKAANKELTFLLFSCPHRLREHLERGRG |
| | | NLEWKEPPSMRLKARPSSPGFSVLTCSAFSFYPP |
| | | ELQLRFLRNGLAAGTGQGDFGPNSDGSFHASSS |
| | | LTVKSGDEHHYCCIVQHAGLAQPLRVELESPAK |
| | | SSVLVVGIVIGVLLLTAAAVGGALLWRRMRSGL |
| | | PAPWISLRGDDTGVLLPTPGEAQDADLKDVNVI |
| | | PATA |
| 8 | Mouse | MGMPLPWALSLLLVLLPQTWGSETRPPLMYHL |
| | FcRn | TAVSNPSTGLPSFWATGWLGPQQYLTYNSLRQE |
| | | ADPCGAWMWENQVSWYWEKETTDLKSKEQLF |
| | | LEALKTLEKILNGTYTLQGLLGCELASDNSSVPT |
| | | AVFALNGEEFMKFNPRIGNWTGEWPETEIVANL |
| | | WMKQPDAARKESEFLLNSCPERLLGHLERGRR |
| | | NLEWKEPPSMRLKARPGNSGSSVLTCAAFSFYP |
| | | PELKFRFLRNGLASGSGNCSTGPNGDGSFHAWS |
| | | LLEVKRGDEHHYQCQVEHEGLAQPLTVDLDSS |
| | | ARSSVPVVGIVLGLLLVVVAIAGGVLLWGRMR |
| | | SGLPAPWLSLSGDDSGDLLPGGNLPPEAEPQGA |
| | | NAFPATS |

In certain embodiments, the FcRn binding polypeptides of the present disclosure comprises a FcRn binding site derived from of the C-terminal region of an immunoglobulin heavy chain polypeptide (e.g. Fc domain). In some embodiments, the Fc polypeptide can comprise two linked Ig-like fold structural domains (e.g. the CH2 and CH3 domains) and at acidic pH a FcRn can bind amino residues in both the CH2 and CH3 structural domains (the FcRn binding site). In some embodiments, the FcRn polypeptide can comprise a Ig-like fold structural domain (e.g. the CH2 of an Fc domain) and at acidic pH a FcRn can bind amino residues of the CH2 structural domain (e.g. the FcRn binding sites). In some embodiments, the FcRn polypeptide can comprise a Ig-like fold structural domains (e.g. CH3 domains of an Fc polypeptide) and at acidic pH a FcRn can bind amino residues of the CH3 structural domain (the FcRn binding sites).

In some embodiments, FcRn binding polypeptides of the disclosure can comprise one or more FcRn binding sites derived from various mammalian species (e.g. from humans) as well various immunoglobulin subtypes, for instance IgG (as non-limiting examples in the case of IgG, IgG1, IgG2, IgG3, IgG4, IgG2a, IgG2d, and/or IgG2c). In some embodiments, the FcRn binding site is or comprises human Fc structural domains, for example, a human IgG Fc structural domain comprising an amino acid sequence that is derived from a human IgG Fc polypeptide sequence. For example, in some embodiments, the FcRn binding site is or comprises human Fc structural domains, comprising an amino acid sequence that is derived from a human IgG1 Fc polypeptide sequence (see SEQ ID NO:9 for the amino acid sequence of wild-type human IgG1 Fc). In other embodiments, the FcRn binding site is or comprises human Fc structural domains, comprising an amino acid sequence that is derived from a human IgG2 Fc polypeptide (see SEQ ID NO:10 for the amino acid sequence of wild-type human IgG2 Fc). In other embodiments, the FcRn binding site is or comprises human Fc structural domains, comprising an amino acid sequence that is derived from a human IgG3 Fc polypeptide (see SEQ ID NO:11 for the amino acid sequence of wild-type human IgG3 Fc). In other embodiments, the FcRn binding site is or comprises human Fc structural domains, comprising an amino acid sequence that is derived from a human IgG4 Fc polypeptide (see SEQ ID NO:12 for the amino acid sequence of wild-type human IgG4 Fc).

In some embodiments, CH2 domains of the FcRn binding polypeptide can be readily obtained from any suitable antibody. Optionally the CH2 domain is of human origin. A CH2 domain may or may not be linked (e.g. at its N-terminus) to a hinge of linker amino acid sequence. In one embodiment, a CH2 domain is a naturally occurring human CH2 domain of IgG1, 2, 4 or 4 subtype. In one embodiment, a CH2 domain is a fragment of a CH2 domain (e.g. of at least 10, 20, 30, 40 or 50 amino acids in length). In one embodiment, a CH2 domain, when present in a polypeptide described herein, will retain binding to a FcRn, particularly human FcRn.

In one aspect, the FcRn binding polypeptide described herein may comprises a Fc domain and said Fc domain exhibits a three dimensional structure that can be superimposed with the Fc structure of a wild type Fc domain of antibody (e.g. IgG). In certain embodiments, the polypeptide described herein comprises a Fe domain and said Fe domain exhibits a three dimensional structure, whose portion between equivalent Ca positions of the beta-strands can be superimposed with a wild type Fe domain of an antibody (e.g. IgG) with root-mean-square deviations (RMSDs) of at most 1, 2, 4, 4, 5, 6, 7, 8, 9, 10 or 15 Å. For example, the structure of the Fe domain can be superimposed with Fe domains of IgG1, IgG2, and IgG4 subtypes as described in Tam S H, et al Antibodies (Basel). 2017; 6(3):12. Methods for comparing two biological structures by calculating the RMSD of superimposed structures are well known in the art (as described in Xu, Y., Xu, D. and Liang, J., 2007. Computational methods for protein structure prediction and modeling. Springer.)

In another aspect, polypeptides for use with the methods and compositions provided herein shall be understood to relate to FcRn binding polypeptide that has at least one mutation, e.g., a substitution, deletion or insertion, as compared to a wild-type immunoglobulin heavy chain Fc polypeptide sequence but retains the overall Ig fold or structure of the native Fe domain.

In some embodiments, the FcRn binding polypeptide of the disclosure comprises modified Fe domains of an immunoglobulin that have the capability of binding the Fc binding site of an FcRn. A modified Fe domain can be at least 50% homologous to any sequence of an Fc portion of any IgG antibody. In some embodiments, an FcRn binding polypeptide can be at least 60% homologous to any sequence of an Fc portion of any IgG antibody. In certain embodiments, a modified Fe domain can be at least 70% homologous to any sequence of an Fc portion of any IgG antibody. In certain embodiments, an modified Fe domain can be at least 80% homologous to any sequence of an Fc portion of any IgG antibody. In certain embodiments, an modified Fe domain can be at least 90% homologous to any sequence of an Fc portion of any IgG antibody.

In some embodiments, a modified Fe domain can be at least 50% homologous to any of SEQ ID NOs.: 13-34. In certain embodiments, an FcRn binding moiety can be at least 60% homologous to any of SEQ ID NOs.: 13-34. In certain embodiments, a modified Fe domain can be at least 70% homologous to any of SEQ ID NOs.: 13-34. In certain embodiments, a modified Fe domain can be at least 80% homologous to any of SEQ ID NOs.:13-34. In certain embodiments, a modified Fe domain can be at least 90% homologous to any of SEQ ID NOs.: 13-34. In certain embodiments, a modified Fe domain can be at least 95% or at least 98% homologous to any of SEQ ID NOs.: 13-34.

In some embodiments, the FcRn binding polypeptide comprises a native FcRn binding site (e.g., Fe domain). In some embodiments, the FcRn binding comprises a modification that alters FcRn binding. In some embodiments, the modified Fc domain of the disclosure is mutated or modified to further enhance FcRn binding. In these embodiments the mutated or modified Fc polypeptide may include the following mutations: M252Y, S254T, T256E, L309N, T250Q, M428L, N434S, N434A, T307A, E380A, using the EU numbering system. In some embodiments, the mutated or modified Fc polypeptide includes one or more mutations selected from the group consisting of M252Y, S256T, T256E, M428L, M428V, N434S, and combinations thereof. Modifications of CH2 and CH3 domains for enhanced FcRn binding are presented in U.S. Ser. No. 16/845,894, hereby incorporated by reference.

In various embodiments, the FcRn binding polypeptides of the disclosure have an increased binding affinity to the Fc binding site of an FcRn at an acidic pH and a decreased binding affinity to the Fc binding site of an FcRn at about neutral pH. In a preferred embodiment, FcRn binding polypeptides of the disclosure have an increased propensity to form a complex with FcRn at an acidic pH (e.g. a pH of 6.5) as opposed to at a neutral pH (e.g. a pH of 7.4)

In some embodiments the equilibrium dissociation constant of the FcRn binding polypeptide bound to FcRn at an acidic pH is at least 10⁻⁴, 10⁻⁵, 10⁻⁶, 10⁻⁷, 10⁻⁸ or 10⁻⁹ M. In some embodiments the equilibrium dissociation constant of the FcRn binding polypeptide bound to FcRn at a pH of 6.5 is equal to the equilibrium dissociation constant of the modified Fc domain bound to FcRn at a pH of 6.5. In some embodiments, equilibrium dissociation constant of the FcRn binding polypeptide bound to FcRn at a pH of 6.5 is increased by at least 5%, 10%, 20%, 30%, 40%, 50% or 60% compared to the equilibrium dissociation constant of the modified Fc domain fragment bound to FcRn.

In some embodiments the equilibrium dissociation constant of the FcRn binding polypeptide bound to FcRn at a neutral pH is above 10⁻⁵, 10⁻⁴, 10⁻³, 10⁻² or 10⁻¹ M. In some embodiments the equilibrium dissociation constant of the FcRn binding polypeptide bound to FcRn at a neutral pH is equal to the equilibrium dissociation constant of the modified Fc domain bound to FcRn at a neutral pH. In some embodiments, equilibrium dissociation constant of the FcRn binding polypeptide bound to FcRn at a pH of 6.5 is increased by at least 20%, 30%, 40%, 50% or 60% compared to the equilibrium dissociation constant of the modified Fc domain fragment bound to FcRn.

In some embodiments, the three-dimensional structure of modified Fc domain of the FcRn binding polypeptide bound to FcRn at an acidic pH has a binding interface that spans a larger surface area (e.g. above 1000 Å) than at physiological pH. In certain embodiments, the buried surface area at the interface between modified Fc domain chain and the polypeptide chains of FcRn can be larger than areas buried at the interface between Fc and other proteins that bind to CH2-CH3 interdomain region of Fc (e,g, protein A, protein G, or rheumatoid factor). Methods for calculating the buried surface area between are well known in the art (as described in Xu, Y., Xu, D. and Liang, J., 2007. Computational methods for protein structure prediction and modeling. Springer.)

In one embodiment of all aspects as described herein, at an acidic pH the FcRn binding polypeptide has binding affinity to FcRn selected from human FcRn, cynomolgus FcRn, mouse FcRn, rat FcRn, sheep FcRn, dog FcRn and rabbit FcRn. In some embodiments, the FcRn binding polypeptide has increased binding affinity to mouse FcRn than to human FcRn.

For the Fc domains of the FcRn binding polypeptide as well as FcRn binding polypeptide disclosed herein, methods for analyzing binding affinity and binding kinetics are known in the art. These methods include, but are not limited to, solid-phase binding assays (e.g., ELISA assay), immunoprecipitation, surface plasmon resonance (e.g., Biacore™) kinetic exclusion assays (e.g., KinExA®), flow cytometry, fluorescence-activated cell sorting (FACS), BioLayer interferometry (e.g., Octet® (ForteBio,)), and Western blot analysis. In some embodiments, ELISA is used to determine binding affinity. Methods for performing ELISA assays are known in the art. In some embodiments, surface plasmon resonance (SPR) is used to determine binding affinity and or binding kinetics. In some embodiments, kinetic exclusion assays are used to determine binding affinity and/or binding kinetics. In some embodiments, BioLayer interferometry assays are used to determine binding affinity and/or binding kinetics.

### 5.2.1 CH2 and CH3 Domain Homodimer Modifications

In various embodiments, the FcRn binding polypeptides described herein are engineered to not form a dimer with another Fe domain (e.g. does not form a homodimer with another modified Fe domain or heterodimer with endogenous Fe domain). In one embodiment, the Fe domains contain modifications to disrupt heterodimerization e.g. by electrostatic engineering of contact residues within a CH3-CH3 interface that are naturally charged, or hydrophobic patch modifications (e.g. does not dimerize via interactions with another CH3 domain, referred to as a monomeric CH3). In certain embodiments herein, specifically the CH3 domain of a Fe domain comprises one or more amino acid modifications (e.g. amino acid substitutions) to disrupt the CH3 dimerization interface. In such embodiments, the CH3 domain modifications will prevent protein scaffold aggregation caused by the exposure of hydrophobic residues when the CH2-CH3 domains are in monomeric form. Simultaneously, the CH3 domain modifications useful in the disclosure will additionally not interfere with the ability of the Fc-derived polypeptide to bind to neonatal Fc receptor (FcRn), e.g. human FcRn.

In one aspect, the FcRn binding polypeptide described herein may comprises a monomeric CH3 domain and said CH3 domain exhibits a three dimensional structure that can be superimposed with the CH3 structure of a wild type CH3 domain of antibody (e.g. IgG). In certain embodiments, the polypeptide described herein comprises monomeric CH3 domain and said monomeric CH3 domain exhibits a three dimensional structure, whose portion between equivalent Ca positions of the beta-strands can be superimposed with a wild type CH3 domain of an antibody (e.g. IgG) with root-mean-square deviations (RMSDs) of at most 1, 2, 4, 4, 5, 6, 7, 8, 9, 10 or 15 Å. For example, the structure of the monomeric CH3 domain can be superimposed with CH3 domains of IgG1, IgG2, and IgG4 subtypes as described in Tam S H, et al Antibodies (Basel). 2017; 6(3):12.

Monomeric modified Fe domains that can be used to prevent homodimer formation have been described in various publications. See, e.g. US 2006/0074225, WO2006/031994, WO2011/063348 and Ying et al. (2012) J. Biol. Chem. 287(23):19399-19407, the disclosures of each of which are incorporated herein by reference. In order to discourage the homodimer formation, one or more residues that make up the CH3-CH3 interface are replaced with a charged amino acid such that the interaction becomes electrostatically unfavorable. For example, WO2011/063348 provides that a positive-charged amino acid in the interface, such as lysine, arginine, or histidine, is replaced with a different (e.g. negative-charged amino acid, such as aspartic acid or glutamic acid), and/or a negative-charged amino acid in the interface is replaced with a different (e.g. positive charged) amino acid. In one embodiment, a CH3 domain described herein comprises an amino acid modification (e.g. substitution) at 1, 2, 3, 4, 5, 6, 7 or 8 of the positions R355, D356, E357, K370, K392, D399, K409, and K439 (according to EU numbering). In certain embodiments, two or more charged residues within the interface are changed to an opposite charge. Exemplary CH3 domains contain K392D and K409D mutations and those comprising D399K and D356K mutations.

A further strategy to maintain monomeric Fc domains comprises replacing one or more large hydrophobic residues that make up the CH3-CH3 interface with a small polar amino acid. Using human IgG as an example, large hydrophobic residues of the CH3-CH3 interface include Y349, L351, L368, L398, V397, F405, and Y407 of an Fc domain. Small polar amino acid residues include asparagine, cysteine, glutamine, serine, and threonine. Thus in one embodiment, a CH3 domain described herein comprises an amino acid modification (e.g. substitution) at 1, 2, 3, 4, 5, 6, 7 or 8 of the positions R355, D356, E357, K370, K392, D399, K409, and K439. In the study described in WO2011/063348, two of the positively charged Lys residues that are closely located at the CH3 domain interface were mutated to Asp. Threonine scanning mutagenesis was then carried out on the structurally conserved large hydrophobic residues in the background of these two Lys to Asp mutations. Fc domains comprising K392D and K409D mutations along with the various substitutions with threonine were analyzed for monomer formation. Exemplary monomeric Fc domains include those having K392D, K409D and Y349T substitutions and those having K392D, K409D and F405T substitutions.

In Ying et al. (2012) J. Biol. Chem. 287(23):19399-19407, amino acid substitutions were made within the CH3 domain at residues L351, T366, L368, P395, F405, T407 and K409. Combinations of different mutations resulted in the disruption of the CH3 dimerization interface, without causing protein aggregation. In one embodiment, a CH3 domain described herein comprises an amino acid modification (e.g. substitution) at 1, 2, 3, 4, 5, 6 or 7 of the positions L351, T366, L368, P395, F405, T407 and/or K409. In one embodiment, a CH3 domain described herein comprises amino acid modifications L351Y, T366Y, L368A, P395R, F405R, T407M and K409A. In one embodiment, a CH3 domain comprises amino acid modifications L351S, T366R, L368H, P395K, F405E, T407K and K409A. In one embodiment, a CH3 domain described herein comprises amino acid modifications L351K, T366S, P395V, F405R, T407A and K409Y.

In various embodiments, the modified Fc domain of the present disclosure demonstrate reduced dimerization as compared to wild-type Fc molecules. Thus, embodiments of the disclosure include compositions comprising a population of FcRn binding polypeptides as described herein, wherein the amount of Fc domain-Fc domain homodimerization exhibited by said FcRn binding polypeptides is less than 10%, less than 20%, less than 30%, less than 40%, less than 50%, less than 60%, less than 70%, less than 80%, less than 90%, less than 95%, less than 97%, or less than 99% of the population. Dimerization may be measured by several techniques known in the art. Preferred methods of measuring homodimerization of the modified Fc domain include Size Exclusion Chromatography (SEC), Analytical Ultra Centrifugation (AUC), Dynamic Light Scattering (DLS), and Native PAGE.

### 5.2.2 Additional Fc Domain Modifications

In some embodiments, FcRn binding polypeptide of the disclosure contains one or more additional modifications. Non-limiting examples of other mutations that can be introduced into the modified Fc domains include, e.g., mutations to increase serum stability and/or half-life, to modulate effector function, to influence glycosylation and or to reduce immunogenicity in humans.

In some embodiments, the FcRn binding polypeptide described herein comprise modifications that reduce effector function, i.e., having a reduced ability to induce certain biological functions upon binding to an Fc receptor (other than FcRn) expressed on or in an effector cell that mediates the effector function. Examples of Fc-Receptor effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. In some embodiments, modified Fc domains present in a FcRn binding polypeptide described herein may include additional modifications that modulate effector function.

In some embodiments, the complement dependent cytotoxicity (CDC) activity of the modified Fc domain; the antibody dependent cell mediated cytotoxicity (ADCC) activity of the modified Fc domain; the antibody dependent cell mediated phagocytosis (ADCP) activity of the modified Fc domain; and/or the antibody dependent intracellular neutralization (ADIN) activity of the modified Fc domain is decreased by at least 10%, 20%, 30%, 40%, or 50% compared to the unmodified Fc domain.

In some embodiments, the complement dependent cytotoxicity (CDC) activity of the modified Fc domain; the antibody dependent cell mediated cytotoxicity (ADCC) activity of the modified Fc domain; the antibody dependent cell mediated phagocytosis (ADCP) activity of the modified Fc domain; and/or the antibody dependent intracellular neutralization (ADIN) activity of the modified Fc domain is decreased by at least 1.5, 2, 3, 4, or 5-fold, compared to the unmodified Fc domain.

In some embodiments, the FcRn binding polypeptide comprises from N-terminus to C-terminus: (a) a modified CH2 domain that is modified relative to the unmodified CH2 domain to decrease effector function; (b) a modified CH3 domain that is modified relative to the unmodified CH3 domain to lack the homodimerize; (c) a linker sequence; and (d) a transmembrane domain.

In some embodiments, the FcRn binding polypeptide comprises from C-terminus to N-terminus: (a) a modified CH3 domain that is modified relative to the unmodified CH3 domain to lack the homodimerize; (b) a modified CH2 domain that is modified relative to the unmodified CH2 domain to decrease effector function; (c) a linker sequence; and (d) a transmembrane domain.

In some embodiments, the FcRn binding polypeptide described herein may comprise modifications that reduce or eliminate effector function. Illustrative modifications include CH2 domain modifications that reduce effector function, which include, but are not limited to, substitutions in the CH2 domain, referred to as modCH2, e.g., at positions 234 and 235, according to the EU numbering scheme. For example, in some embodiments, modified Fc domain can comprise alanine residues at positions 234 and 235. Thus, FcRn binding polypeptides may have L234A and L235A substitutions.

In one embodiment, a CH2 domain, when present in a FcRn binding polypeptide described herein, confers decreased or lack of binding to a Fcγ receptor, notably FcγRIIIA (CD16). FcRn binding polypeptides that comprise a CH2 domain that cannot bind CD16 will not be capable of activating or mediating ADCC by cells (e.g. NK cells, T cells) that do not express the effector cell antigen of interest (e.g. NKp46, CD3, etc.).

In one embodiment, a CH2 domain, when present in a FcRn binding polypeptide described herein, will have decreased or will substantially lack antibody dependent cytotoxicity (ADCC), complement dependent cytotoxicity (CDC), antibody dependent cellular phagocytosis (ADCP), FcR-mediated cellular activation (e.g. cytokine release through FcR cross-linking), and/or FcR-mediated platelet activation/depletion.

In one embodiment, a CH2 domain, when present in a FcRn binding polypeptide described herein, has substantial loss of binding to activating Fcγ receptors, e.g., FcγRIIIA (CD16), FcγRIIA (CD32A) or CD64, or to an inhibitory Fc receptor, e.g., FcγRIIB (CD32B). In one embodiment, a CH2 domain, when present in a FcRn binding polypeptide described herein, furthermore has substantial loss of binding to the first component of complement (C1q).

For example, substitutions into the CH2 domain of human IgG1 of IgG2 residues at positions 233-236 and IgG4 residues at positions 327, 330 and 331 were shown to greatly reduce binding to Fcγ receptors and thus ADCC and CDC. Furthermore, Idusogie et al. (2000) J Immunol. 164(8):4178-84 demonstrated that alanine substitution at different positions, including K322, significantly reduced complement activation.

Additional CH2 domain modifications or mutations that modulate an effector function include, but are not limited to, the following: position 329 may have a mutation in which proline is substituted with a glycine or arginine or an amino acid residue large enough to destroy the Fc/Fcγ receptor interface that is formed between proline 329 of the Fc and tryptophan residues Trp 87 and Trp 110 of FcγRIII.

Additional illustrative substitutions in the CH2 domain include S228P, E233P, L235E, N297A, N297D, and P331S, according to the EU numbering scheme. Multiple substitutions may also be present, e.g., L234A and L235A of a human IgG1 Fc region; L234A, L235A, and P329G of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, according to the EU numbering scheme. In some embodiments, one FcRn binding polypeptides may have one or more amino acid substitutions that modulate ADCC, e.g., substitutions at positions 298, 333, and/or 334, according to the EU numbering scheme.

In one embodiment, the FcRn binding polypeptide has decreased binding to a human Fcγ receptor (e.g. CD16, CD32A, CD32B and/or CD64), e.g., compared to a full length wild type human IgG1 Fc domain. In one embodiment, the polypeptide has decreased (e.g. partial or complete loss of) antibody dependent cytotoxicity (ADCC), complement dependent cytotoxicity (CDC), antibody dependent cellular phagocytosis (ADCP), Fc receptor mediated cellular activation (e.g. cytokine release through FcR cross-linking), and/or Fc receptor mediated platelet activation/depletion, as mediated by immune effector cells, compared, e.g., to a FcRn binding polypeptide having a wild-type Fc domain of human IgG1 isotype.

In one embodiment, a CH2 domain that retains binding to a FcRn receptor but has reduction of binding to Fcγ receptors lacks or has modified N-linked glycosylation, e.g. at residue N297 according to the EU numbering scheme. For example, the FcRn binding polypeptide can be expressed in a cell line which naturally has a high enzyme activity for adding fucosyl to the N-acetylglucosamine which does not yield glycosylation at N297. In another embodiment, a CH2 domain may have one or more substitutions that result in lack of the canonical Asn-X-Ser/Thr N-linked glycosylation motif at residues 297-299, which can also result in reduction of binding to Fcγ receptors. Thus, a CH2 domain may have a substitution at N297 and/or at neighboring residues (e.g. 298, 299).

In one embodiment, a FcRn binding polypeptide contains a CH2 domain derived from an IgG2 Fc mutant exhibiting diminished FcγR binding capacity but having conserved FcRn binding. In certain embodiments, FcRn binding polypeptide comprises the mutations V234A, G237A, P238S according to the EU numbering system. In another aspect, the FcRn binding polypeptide comprises mutations V234A, G237A, H268Q or H268A, V309L, A330S, P331S according to the EU numbering system. In a particular aspect, the FcRn binding polypeptide contains a CH2 domain derived from an IgG2 Fc comprising mutations V234A, G237A, P238S, H268A, V309L, A330S, P331S, and, optionally, P233S according to the EU numbering system.

In one embodiment, the FcRn binding polypeptide comprises at least one amino acid modification (for example, possessing 1, 2, 3, 4, 5, 6, 7, 8, 9, or more amino acid modifications) in the CH2 domain of the Fc domain, optionally further in combination with one or more amino acid modification in other domains (e.g. the CH3 domain). Any combination of Fc domain modifications can be made. In one embodiment, a FcRn binding polypeptide of the disclosure which has decreased binding to a human Fcγ receptor comprises at least one amino acid modification (for example, possessing 1, 2, 3, 4, 5, 6, 7, 8, 9, or more amino acid modifications) relative to a wild-type CH2 domain within amino acid residues 237-340 (EU numbering), such that the FcRn fusion polypeptide comprising such CH2 domain has decreased affinity for a human Fcγ receptor of interest relative to an equivalent polypeptide comprising a wild-type CH2 domain.

In one aspect, the FcRn binding polypeptide described herein may comprises a CH2 domain (e.g., modified CH2 domain) and said CH2 domain (e.g., modified CH2 domain) exhibits a three dimensional structure that can be superimposed with the CH2 structure of a wild type CH2 domain of antibody (e.g. IgG). In certain embodiments, the polypeptide described herein comprises modified CH2 domain and said modified CH2 domain exhibits a three dimensional structure, whose portion between equivalent Ca positions of the beta-strands can be superimposed with a wild type CH2 domain of an antibody (e.g. IgG) with root-mean-square deviations (RMSDs) of at most 1, 2, 4, 4, 5, 6, 7, 8, 9, 10 or 15 Å. For example, the structure of the modified CH2 domain can be superimposed with CH2 domains of IgG1, IgG2, and IgG4 subtypes as described in Tam S H, et al Antibodies (Basel). 2017; 6(3):12.

In some embodiments, the FcRn binding polypeptide comprising a CH3 domain described herein may comprise modifications that reduce activation of Tripartite motif-containing protein 21 (TRIM21). Illustrative CH3 domain mutations that reduce activation of TRIM21 include, but are not limited to, substitutions in the CH3 domain, e.g. at position 433, according to the EU numbering scheme. For example, in some embodiments, the CH3 domain can comprise alanine residues at position 433 according to the EU numbering scheme.

In certain aspects, provided herein are FcRn binding polypeptides anchored to a signal neutral protein scaffold in nanovesicles (e.g., EVs and hybridosomes) for attaching molecules of interest.

In a further aspect, the FcRn binding site of an Fc polypeptide useful for the disclosure may involve internal amino-acids close to the inter-domain interface (e.g. hinge region) between CH2 and CH3 domains while not involving C- or N-terminus of CH2 and CH3 domains (e.g. a fc polypeptide). Hence, in certain embodiments, both termini of the structural domains comprising an FcRn binding site are not relevant to the FcRn binding function, and therefore can be modified (e.g. linked to a heterologous protein) without significantly altering the FcRn binding function of the polypeptide. Polypeptides comprising domains with an internal FcRn binding site (distal from the N- or C-terminal) that is accessible for structural complementation (e.g. FcRn binding) may provide an design advantage when tethered to either N- or C-terminal of a scaffold protein comprising different types of transmembrane domains (Type 1, II and PT) as described in section). Furthermore, provided herein are polypeptides comprising internal FcRn binding sites with accessible N- and C-termini that be fused to a scaffold protein and optionally a heterologous protein for additional functionalities such as cell type-specific targeting, receptor decoys, or purification. In some embodiments, the FcRn binding site is fused to a protein scaffold that protrudes from membrane, thereby allowing FcRn access to FcRn binding site. In certain embodiments, this results in long protrusion of the FcRn binding site from the membrane (e.g. when fused to Ephrin receptor scaffold protein) which are simultaneously flexible to bend and/or reconfigure while maintaining stability. A stable membrane anchoring can streamline the configuration of the resulting fusion protein, in that the molecule of interest may be directed to the outer surface of a nanovesicle (e.g., an EV or hybridosome) or cell.

In some embodiments, the FcRn binding polypeptide described herein does not substantially bind to C1q, FcγRI, FcγRII or FcγRIII.

TABLE 1
Wild-type IgG1 Fc domain and Examples of modified Fc domains

| Seq | | |
|---|---|---|
| ID | Name | Sequence |
| 9 | IgG1 | APELLGGPSVFLFPPKPKDTLMISRTPEVTCVVVDVSHEDPEVKFN |
| | wt | WYVDGVEVHNAKTKPREEQYNSTYRVVSVLTVLHQDWLNGKEY |
| | | KCKVSNKALPAPIEKTISKAKGQPREPQVYTLPPSRDELTKNQVSL |
| | | TCLVKGFYPSDIAVEWESNGQPENNYKTTPPVLDSDGSFFLYSKLT |
| | | VDKSRWQQGNVFSCSVMHEALHNHYTQKSLSLSPGK |
| 10 | IgG2 | APPVAGPSVFLFPPKPKDTLMISRTPEVTCVVVDVSHEDPEVQFN |
| | wt | WYVDGVEVHNAKTKPREEQFNSTFRVVSVLTVVHQDWLNGKEY |
| | | KCKVSNKGLPAPIEKTISKTKGQPREPQVYTLPPSREEMTKNQVSL |
| | | TCLVKGFYPSDISVEWESNGQPENNYKTTPPMLDSDGSFFLYSKLT |
| | | VDKSRWQQGNVFSCSVMHEALHNHYTQKSLSLSPGK |
| 11 | IgG3 | APELLGGPSVFLFPPKPKDTLMISRTPEVTCVVVDVSHEDPEVQFK |
| | wt | WYVDGVEVHNAKTKPREEQYNSTFRVVSVLTVLHQDWLNGKEY |
| | | KCKVSNKALPAPIEKTISKTKGQPREPQVYTLPPSREEMTKNQVSL |
| | | TCLVKGFYPSDIAVEWESSGQPENNYNTTPPMLDSDGSFFLYSKLT |
| | | VDKSRWQQGNIFSCSVMHEALHNRFTQKSLSLSPG |
| 12 | IgG4 | APEFLGGPSVFLFPPKPKDTLMISRTPEVTCVVVDVSQEDPEVQFN |
| | wt | WYVDGVEVHNAKTKPREEQFNSTYRVVSVLTVLHQDWLNGKEY |
| | | KCKVSNKGLPSSIEKTISKAKGQPREPQVYTLPPSQEEMTKNQVSL |
| | | TCLVKGFYPSDIAVEWESNGQPENNYKTTPPVLDSDGSFFLYSRLT |
| | | VDKSRWQEGNVFSCSVMHEALHNHYTQKSLSLSLGK |
| 13 | modFC_ | APEAAGGPSVFLFPPKPKDTLMISRTPEVTCVVVDVSHEDPEVKFN |
| | 1 | WYVDGVEVHNAKTKPREEQYGSTYRVVSVLTVLHQDWLNGKEY |
| | | KCAVSNKALAAPIEKTISKAKGQPREPQVYTLPPSRDELTKNQVSL |
| | | TCLVKGFYPSDIAVEWESNGQPENNYKTTPPVLDSDGSFFLYSKLT |
| | | VDKSRWQQGNVFSCSVMHEALANHYTQKSLSLSPGK |
| 14 | modFC_ | APEAAGGPSVFLFPPKPKDTLMISRTPEVTCVVVDVSHEDPEVKFN |
| | 2 | WYVDGVEVHNAKTKPREEQYGSTYRVVSVLTVLHQDWLNGKEY |
| | | KCAVSNKALAAPIEKTISKAKGQPREPQVYTSPPSRDELTKNQVSL |
| | | RCHVKGFYPSDIAVEWESNGQPENNYKTTKPVLDSDGSFELKSAL |
| | | TVDKSRWQQGNVFSCSVMHEALANHYTQKSLSLSPGK |
| 15 | modFC_ | APEAAGGPSVFLFPPKPKDTLMISRTPEVTCVVVDVSHEDPEVKFN |
| | 3 | WYVDGVEVHNAKTKPREEQYGSTYRVVSVLTVLHQDWLNGKEY |
| | | KCAVSNKALAAPIEKTISKAKGQPREPQVYTYPPSRDELTKNQVSL |
| | | YCAVKGFYPSDIAVEWESNGQPENNYKTRPPVLDSDGSFRLMSAL |
| | | TVDKSRWQQGNVFSCSVMHEALANHYTQKSLSLSPGK |
| 16 | modFC_ | APEAAGGPSVFLFPPKPKDTLMISRTPEVTCVVVDVSHEDPEVKFN |
| | 4 | WYVDGVEVHNAKTKPREEQYGSTYRVVSVLTVLHQDWLNGKEY |
| | | KCAVSNKALAAPIEKTISKAKGQPREPQVYTSPPSRDELTKNQVSL |
| | | RCHVKGFYPSDIAVEWESNGQPENNYKTTKPVLDSDGSFFLYSKL |
| | | TVDKSRWQQGNVFSCSVMHEALANHYTQKSLSLSPGK |
| 17 | modFC_ | APELLGGPSVFCFPPKPKDTLMISRTPEVTCVVVDVSHEDPEVKFN |
| | 5 | WYVDGVEVHNAKTKPREEQYNSTYRVVSVLTVLHQDWLNGKEY |
| | | KCKVSNKALPAPIECTISKAKGQPREPQVYTSPPSRDELTKNQVSL |
| | | RCHVKGFYPSDIAVEWESNGQPENNYKTTKPVLDSDGSFFLYSKL |
| | | TVDKSRWQQGNVFSCSVMHEALHNHYTQKSLSLSPGK |
| 18 | modFC_ | APEAAGGPSVFLFPPKPKDTLYITREPEVTCVVVDVSHEDPEVKFN |
| | 6 | WYVDGVEVHNAKTKPREEQYGSTYRVVSVLTVLHQDWLNGKEY |
| | | KCAVSNKALAAPIEKTISKAKGQPREPQVYTSPPSRDELTKNQVSL |
| | | RCHVKGFYPSDIAVEWESNGQPENNYKTTKPVLDSDGSFFLYSKL |
| | | TVDKSRWQQGNVFSCSVMHEALANHYTQKSLSLSPGK |
| 19 | modFC_ | APELLGGPSVFLFPPKPKDTLYITREPEVTCVVVDVSHEDPEVKFN |
| | 7 | WYVDGVEVHNAKTKPREEQYNSTYRVVSVLTVLHQDWLNGKEY |
| | | KCKVSNKALPAPIEKTISKAKGQPREPQVYTSPPSRDELTKNQVSL |
| | | RCHVKGFYPSDIAVEWESNGQPENNYKTTKPVLDSDGSFFLYSKL |
| | | TVDKSRWQQGNVFSCSVMHEALHNHYTQKSLSLSPGK |
| 20 | modFC_ | APEAAGGPSVFLFPPKPKDTLYITREPEVTCVVVDVSHEDPEVKFN |
| | 8 | WYVDGVEVHNAKTKPREEQYGSTYRVVSVLAVLHQDWLNGKE |
| | | YKCAVSNKALAAPIEKTISKAKGQPREPQVYTSPPSRDELTKNQVS |
| | | LRCHVKGFYPSDIAVAWESNGQPENNYKTTKPVLDSDGSFFLYSK |
| | | LTVDKSRWQQGNVFSCSVMHEALAAHYTQKSLSLSPGK |
| 21 | modFC_ | APEAAGGPSVFLFPPKPKDTLMISRDPEVTCVVVDVSHEDPEVKFN |
| | 9 | WYVDGVEVHNAKTKPREEQYGSTYRVVSVLRVLHVDWLNGKEY |
| | | KCAVSNKALAAPIEKTISKAKGQPREPQVYTSPPSRDELTKNQVSL |
| | | RCHVKGFYPSDIAVEWESNGQPENNYKTTKPVLDSDGSFFLYSKL |
| | | TVDKSRWQQGNVFSCSVMHEALANHYTQKSLSLSPGK |
| 22 | modFC_ | APEAAGGPSVFLFPPKPKDTLMISRDPEVTCVVVDVSHEDPEVKFN |
| | 10 | WYVDGVEVHNAKTKPREEQYGSTYRVVSVLTVLHQDWLDGKEY |
| | | KCAVSNKALAAPIEKTISKAKGQPREPQVYTSPPSRDELTKNQVSL |
| | | RCHVKGFYPSDIVVEWESNGQPENNYKTTKPVLDSDGSFFLYSKL |
| | | TVDKSRWQQGNVFSCSVMHEALANHYTQKSLSLSPGK |
| 23 | modFC_ | APEAAGGPSVFLFPPKPKDTLMISRDPEVTCVVVDVSHEDPEVKFN |
| | 11 | WYVDGVEVHDAKTKPREEQYGSTYRVVSVLRVLHVDWLNGKEY |
| | | KCAVSNKALAAPIEKTISKAKGQPREPQVYTSPPSRDELTKNQVSL |
| | | RCHVKGFYPSDIAVEWESNGQPENNYKTTKPVLDSDGSFFLYSKL |
| | | TVDKSRWQQGNVFSCSVMHEALANHYTQKSLSLSPGK |
| 24 | modFC_ | APEAAGGPSVFLFPPKPKDTLMISRTPEVTCVVVDVSHEDPEVKFN |
| | 12 | WYVDGVEVNNAKTKPREEQYGSTYRVVSVLQVLHQDWLDGKE |
| | | YKCAVSNKALAAPIEKTISKAKGQPREPQVYTSPPSRDELTKNQVS |
| | | LRCHVKGFYPSDIAVEWESNGQPENNYKTTKPVLDSDGSFFLYSK |
| | | LTVDKSRWQQGNVFSCSVMHEALANHYTQKSLSLSPGK |
| 25 | modFC_ | APEAAGGPSVFLFPPKPKDTLMISRDPEVTCVVVDVSHEDPEVKFN |
| | 13 | WYVDGVEVHNAKTKPREEQYGSTYRVVSVLRVLHVDWLNGKEY |
| | | KCAVSNKALAAPIEKTISKAKGQPREPQVYTSPPSRDELTKNQVSL |
| | | RCHVKGFYPSDIVVEWESNGQPENNYKTTKPVLDSDGSFFLYSKL |
| | | TVDKSRWQQGNVFSCSVMHEALANHYTQKSLSLSPGK |
| 26 | modFC_ | APEAAGGPSVFLFPPKPKDTLMISRTPEVTCVVVDVSHEDPEVKFN |
| | 14 | WYVDGVEVDNAKTKPREEQYGSTYRVVSVLTVLHVDWLNGKEY |
| | | KCAVSNKALAAPIEKTISKAKGQPREPQVYTSPPSRDELTKNQVSL |
| | | RCHVKGFYPSDIVVEWESNGQPENNYKTTKPVLDSDGSFFLYSKL |
| | | TVDKSRWQQGNVFSCSVMHEALANHYTQKSLSLSPGK |
| 27 | modFC_ | APEAAGGPSVFLFPPKPKDTLMISRDPEVTCVVVDVSHEDPEVKFN |
| | 15 | WYVDGVEVDNAKTKPREEQYGSTYRVVSVLTVLHQDWLNGKEY |
| | | KCAVSNKALAAPIEKTISKAKGQPREPQVYTSPPSRDELTKNQVSL |
| | | RCHVKGFYPSDIVVEWESNGQPENNYKTTKPVLDSDGSFFLYSKL |
| | | TVDKSRWQQGNVFSCSVMHEALANHYTQKSLSLSPGK |
| 28 | modFC_ | APEAAGGPSVFLFPPKPKDTLMISRDPEVTCVVVDVSHEDPEVKFN |
| | 16 | WYVDGVEVHNAKTKPREEQYGSTYRVVSVLTVLHVDWLNGKEY |
| | | KCAVSNKALAAPIEKTISKAKGQPREPQVYTSPPSRDELTKNQVSL |
| | | RCHVKGFYPSDIVVEWESNGQPENNYKTTKPVLDSDGSFFLYSKL |
| | | TVDKSRWQQGNVFSCSVMHEALANHYTQKSLSLSPGK |
| 29 | modFC_ | APEAAGGPSVFLFPPKPKDTLMISRDPEVTCVVVDVSHEDPEVKFN |
| | 17 | WYVDGVEVDDAKTKPREEQYGSTYRVVSVLRVLHQDWLNGKEY |
| | | KCAVSNKALAAPIEKTISKAKGQPREPQVYTSPPSRDELTKNQVSL |
| | | RCHVKGFYPSDIVVEWESNGQPENNYKTTKPVLDSDGSFFLYSKL |
| | | TVDKSRWQQGNVFSCSVMHEALANHYTQKSLSLSPGK |
| 30 | modFC_ | APEAAGGPSVFLFPPKPKDTLMISRDPEVTCVVVDVSHEDPEVKFN |
| | 18 | WYVDGVEVDNAKTKPREEQYGSTYRVVSVLRVLHVDWLNGKEY |
| | | KCAVSNKALAAPIEKTISKAKGQPREPQVYTSPPSRDELTKNQVSL |
| | | RCHVKGFYPSDIVVEWESNGQPENNYKTTKPVLDSDGSFFLYSKL |
| | | TVDKSRWQQGNVFSCSVMHEALANHYTQKSLSLSPGK |
| 31 | modFC_ | APEAAGGPSVFLFPPKPKDTLMISRTPEVTCVVVDVSHEDPEVKFN |
| | 19 | WYVDGVEVHNAKTKPREEQYGSTYRVVSVLQVLHVDWLNGKE |
| | | YKCAVSNKALAAPIEKTISKAKGQPREPQVYTSPPSRDELTKNQVS |
| | | LRCHVKGFYPSDIVVEWESNGQPENNYKTTKPVLDSDGSFFLYSK |
| | | LTVDKSRWQQGNVFSCSVMHEALANHYTQKSLSLSPGK |
| 32 | modFC_ | APEAAGGPSVFLFPPKPKDTLMISRTPEVTCVVVDVSHEDPEVKFN |
| | 20 | WYVDGVEVDNAKTKPREEQYGSTYRVVSVLQVLHQDWLNGKE |
| | | YKCAVSNKALAAPIEKTISKAKGQPREPQVYTSPPSRDELTKNQVS |
| | | LRCHVKGFYPSDIVVEWESNGQPENNYKTTKPVLDSDGSFFLYSK |
| | | LTVDKSRWQQGNVFSCSVMHEALANHYTQKSLSLSPGK |
| 33 | modFC_ | APEAAGGPSVFLFPPKPKDTLMISRDPEVTCVVVDVSHEDPEVKFN |
| | 21 | WYVDGVEVDNAKTKPREEQYGSTYRVVSVLRVLHVDWLNGKEY |
| | | KCAVSNKALAAPIEKTISKAKGQPREPQVYTSPPSRDELTKNQVSL |
| | | RCHVKGFYPSDIVVEWESNGQPENNYKTTKPVLDSDGSFFLYSKL |
| | | TVDKSRWQQGNVFSCSVMHEALANHYTQKSLSLSPGK |
| 34 | modFC_ | APEAAGGPSVFLFPPKPKDTLMISRTPEVTCVVVDVSHEDPEVKFN |
| | 22 | WYVDGVEVHNAKTKPREEQYNSTYRVVSVLTVLHQDWLNGKEY |
| | | KCKVSNKALPAPIEKTISKAKGQPREPQVYTKSPSRDELTKNQVSL |
| | | SCHVKGFYPSDIAVLWESYGTEWSSYKTTVPVLDSDGSFRLASYL |
| | | TVTKEEWQQGFVFSCSVMHEALHNHYTQKSLSLSPGK |

### 5.2.3 Architecture of FcRn Binding Polypeptides

In one aspect the FcRn binding polypeptide comprises a FcRn binding site linked to a scaffold protein comprising a transmembrane domain (e.g. scaffold protein). In some embodiments the FcRn binding site comprises modified Fc domain of an immunoglobulin that have the capability of binding the Fc binding site of an FcRn.

(a) Transmembrane Domains (e.g. Scaffold Proteins Containing Transmembrane Domains)

In various embodiments, FcRn binding polypeptides of the disclosure comprise a transmembrane domain of membrane-bound proteins or transmembrane proteins that comprise one or more transmembrane regions that are embedded in and traverse at least once a cellular membrane. Such a transmembrane region or a functional fragment thereof may be used as membrane anchors of a FcRn binding polypeptide. A transmembrane domain useful in a FcRn binding polypeptide of the disclosure may originate from a transmembrane protein that is associated with any of a variety of membranes of a cell including, but not limited to, a plasma membrane, an endoplasmic reticulum membrane, a Golgi complex membrane, a lysosomal membrane, a nuclear membrane, and a mitochondrial membrane. Examples of transmembrane protein associated with any of these different types of membranes are routinely found in proteomics data sets of EV samples (e.g. www.exocarta.org) and in some cases, when endocytosis signals of transmembrane proteins such as CD63 are mutated to divert localization from the endosome membrane to the plasma membrane, resulting secreted EVs contain higher amounts of diverted transmembrane proteins than endosome-targeted forms of the same proteins (see Fordjour et al, bioRxiv; 2019).

There are four general classes or types of transmembrane proteins (Types I-IV, see, Nelson and Cox, Principles of Biochemistry (2008)). A Type I transmembrane protein has its N-terminal region targeted to the endoplasmic reticulum (ER) lumen and its C-terminal region directed to the cytoplasm. A type II transmembrane protein has its N-terminal region targeted to the cytoplasmic domain and its C-terminal region directed to the ER lumen. A PT type transmembrane protein is a “multi-pass” or polytopic transmembrane protein that has more than one segment of the translated protein that spans the cellular membrane.

In various embodiments, the transmembrane domain in a FcRn binding polypeptide of the disclosure comprises all or part of a transmembrane region of a transmembrane protein that normally traverses the membrane of a cell with which the transmembrane protein is normally associated. The transmembrane domain of a FcRn binding polypeptide of the disclosure may comprise not only a membrane-spanning region of a transmembrane protein but also additional amino acids of the transmembrane protein that are located in flanking regions, either upstream (N-terminal) and/or downstream (C-terminal) to the membrane-spanning or membrane-embedded region of the transmembrane protein. For example, in particular embodiments, the entire transmembrane region of a transmembrane protein will be used. In additional embodiments, the entire transmembrane region and all or part of any upstream or downstream region of the membrane-embedded portion of a transmembrane protein may be used as the transmembrane domain of a FcRn binding polypeptide according to the disclosure. Additional amino acids located either upstream (N-terminal) and/or downstream (C-terminal) from the membrane-embedded portion of a transmembrane protein that may be part of a transmembrane anchor of a FcRn binding polypeptide of the disclosure may have a range of sizes including, but not limited to, 1 to 10 amino acids, 1 to 20 amino acids, 1 to 30 amino acids, 1 to 40 amino acids, 1 to 50 amino acids, 1 to 60 amino acids, 1 to 70 amino acids, 1 to 80 amino acids, 1 to 90 amino acids, 1 to 100 amino acids, 1 to 200 amino acids, 1 to 300 amino acids, 1 to 400 amino acids, 1 to 500 amino acids, 1 to 600 amino acids, 1 to 700 amino acids, 1 to 800 amino acids, and 1 to 900 amino acids. In some embodiments, a fragment transmembrane domain lacks at least 5, 10, 50, 100, 200, 300, 400, 500, 600, 700, or 800 amino acids from the N-terminus of the native protein. In some embodiments, a fragment transmembrane domain lacks at least 5, 10, 50, 100, 200, 300, 400, 500, 600, 700, or 800 amino acids from the C-terminus of the native protein. In some embodiments, the sequence encodes a fragment of the transmembrane domain lacking at least 5, 10, 50, 100, 200, 300, 400, 500, 600, 700, or 800 amino acids from both the N-terminus and C-terminus of the native protein. In some embodiments, the sequence encodes a fragment of the transmembrane domain protein lacking one or more functional or structural domains of the native protein.

The FcRn binding polypeptide comprising a transmembrane domain described herein, may also comprise the entire cytoplasmic region attached to a transmembrane region of a transmembrane protein or a truncation of the cytoplasmic region by one or more amino acids, for example, to eliminate an undesired signaling function of the cytoplasmic tail. For example, the presence of a kinase domain in the C-terminal portion of a cytoplasmic region of a transmembrane protein can serve as a signaling domain. Accordingly, if the membrane-embedded (transmembrane) region and all or part of the adjacent cytoplasmic C-terminal region of a kinase transmembrane protein is to be used as a transmembrane domain of a fusion protein of the disclosure, any known functional kinase signal can be eliminated or disrupted so that a fusion protein comprising the transmembrane region and any adjacent cytoplasmic does not activate the host cell.

Tables 2-4 below, provide a list of several non-limiting examples of scaffold proteins comprising single-pass (Table 2) and multi-pass (Table 3 and Table 4) transmembrane domains along with the Uniprot Database entries. A transmembrane domain that may be used in FcRn-binding polypeptide of the disclosure, can use a part of the transmembrane region sequence sufficient to anchor the FcRn binding polypeptide to a nanovesicle. Other portions of the transmembrane protein, including segments of the flanking regions upstream or downstream of the transmembrane region may be included in the FcRn binding polypeptides, so long as their inclusion enhances, or at least does not significantly diminish the display of the FcRn binding polypeptide on the surface of nanovesicles. Fusing a polypeptide comprising a FcRn binding site to the surface accessible end of a scaffold protein comprising a transmembrane domain can yield a structure which is flexible to bend and/or reconfigure but at the same time stable. Moreover, in some embodiments, the ectodomain of a scaffold protein may provide a long protrusion for reach, as the ectodomain of the scaffold protein protrudes from the membrane. In some embodiments, FcRn binding polypeptide does not comprise a transmembrane domain of Seq ID No.: 35.
(GLWTTITIFITLFLLSVCYSATVTFF) (e.g. the transmembrane domain of a membrane bound IgG).

TABLE 2
Examples of single-pass (bitopic) transmembrane
proteins that can be used as scaffold proteins

| Uniprot | | |
|---|---|---|
| Entry | Gene | Protein name |
| Q8NI60 | ADCK3 | Atypical kinase |
| P35590 | TIE1 | Tyrosine-protein kinase receptor |
| Q15256 | PTPRR | Receptor-type tyrosine-protein phosphatase R |
| Q9P2B2 | PTGFRN | Prostaglandin F2 receptor negative regulator |
| Q9Y4D7 | PLXND1 | Plexin-D1 |
| Q969P0 | IGSF8 | Immunoglobulin superfamily member 8 |
| P22607 | FGFR3 | Fibroblast growth factor receptor 3 |
| P37173 | TGFBR2 | TGF-beta receptor type-2 |
| P35613 | BSG | Basigin |
| P08195 | SLC3A2 | 4F2 cell-surface antigen heavy chain |
| Q9P2E7 | PCDH10 | Protocadherin-10 |
| Q9NZ08 | ERAP1 | Endoplasmic reticulum aminopeptidase 1 |
| P20645 | M6PR | Cation-dependent mannose-6-phosphate receptor |
| P16234 | PDGFRA | Platelet-derived growth factor receptor alpha |
| P09619 | PDGFRB | Platelet-derived growth factor receptor beta |
| P54709 | ATP1B3 | Sodium/potassium-transporting ATPase subunit beta-3 |
| P05556 | ITGB1 | Integrin beta-1 |
| Q6ZU64 | CCDC108 | Cilia- and flagella-associated protein 65 |
| Q9UHE5 | GLA | N-acetyltransferase 8 |
| O43464 | HTRA2 | Serine protease HTRA2, mitochondrial |
| P23467 | PTPRB | Receptor-type tyrosine-protein phosphatase beta |
| O95297 | MPZL1 | Myelin protein zero-like protein 1 |
| P13473 | LAMP2 | Lysosome-associated membrane glycoprotein 2 |
| P49755 | TMED10 | Transmembrane emp24 domain-containing protein 10 |
| Q8NBZ7 | UXS1 | UDP-glucuronic acid decarboxylase 1 |
| Q9Y662 | HS3ST3B1 | Heparan sulfate glucosamine 3-O-sulfotransferase 3B1 |
| Q14126 | DSG2 | Desmoglein-2 |
| P11717 | IGF2R | Cation-independent mannose-6-phosphate receptor |
| O94991 | SLITRK5 | SLIT and NTRK-like protein 5 |
| P42167 | TMPO | Lamina-associated polypeptide 2 |
| P02786 | TFRC | Transferrin receptor protein 1 |
| O75787 | ATP6AP2 | Renin receptor |
| Q12907 | LMAN2 | Vesicular integral-membrane protein VIP36 |
| Q86X29 | LSR | Lipolysis-stimulated lipoprotein receptor |
| Q8NDV1 | ST6GALNAC3 | Alpha-N-acetylgalactosaminide alpha-2,6-sialyltransferase 3 |
| Q7Z7H5 | TMED4 | Transmembrane emp24 domain-containing protein 4 |
| Q9BVK6 | TMED9 | Transmembrane emp24 domain-containing protein 9 |
| O60513 | B4GALT4 | Beta-1,4-galactosyltransferase 4 |
| Q8NCH0 | CHST14 | Carbohydrate sulfotransferase 14 |
| O15162 | PLSCR1 | Phospholipid scramblase 1 |
| P05026 | ATP1B1 | Sodium/potassium-transporting ATPase subunit beta-1 |
| P27824 | CANX | Calnexin |
| O95196 | CSPG5 | Chondroitin sulfate proteoglycan 5 |
| P10321 | HLA-C | HLA class I histocompatibility antigen, C alpha chain |
| P04439 | HLA-A | HLA class I histocompatibility antigen, A alpha chain |
| P98172 | EFNB1 | Ephrin-B1 |
| Q14703 | MBTPS1 | Membrane-bound transcription factor site-1 protease |
| P53801 | PTTG1IP | Pituitary tumor-transforming gene 1 protein-interacting |
| | | protein |
| Q9NPF2 | CHST11 | Carbohydrate sulfotransferase 11 |
| Q9NRB3 | CHST12 | Carbohydrate sulfotransferase 12 |
| P78310 | CXADR | Coxsackievirus and adenovirus receptor |
| P19022 | CDH2 | Cadherin-2 |
| P06756 | ITGAV | Integrin alpha-V |
| Q13308 | PTK7 | Inactive tyrosine-protein kinase 7 |
| Q92824 | PCSK5 | Proprotein convertase subtilisin/kexin type 5 |
| Q9BX67 | JAM3 | Junctional adhesion molecule C |
| O43505 | B3GNT1 | Beta-1,4-glucuronyltransferase 1 |
| Q9NY97 | B3GNT1 | N-acetyllactosaminide-beta-1,3-N- |
| | | acetylglucosaminyltransferase 2 |
| Q8IZ52 | CHPF | Chondroitin sulfate synthase 2 |
| Q96MM7 | HS6ST2 | Heparan-sulfate 6-O-sulfotransferase 2 |
| O00391 | QSOX1 | Sulfhydryl oxidase 1 |
| O94766 | B3GAT3 | Galactosylgalactosylxylosylprotein-3-beta- |
| | | glucuronosyltransferase 3 |
| P78410 | BTF3 | Butyrophilin subfamily 3 member A2 |
| O00478 | BTF3 | Butyrophilin subfamily 3 member A3 |
| P08648 | ITGA5 | Integrin alpha-5 |
| Q99523 | SORT1 | Sortilin |
| O60499 | STX10 | Syntaxin-10 |
| Q5ZPR3 | CD276 | CD276 antigen |
| O75054 | IGSF3 | Immunoglobulin superfamily member 3 |
| P05106 | ITGB3 | Integrin beta-3 |
| Q92542 | NCSTN | Nicastrin |
| P13224 | GP1BB | Platelet glycoprotein Ib beta chain |
| Q9H6X2 | ATR | Anthrax toxin receptor 1 |
| Q15904 | ATP6AP1 | V-type proton ATPase subunit S1 |
| Q68D85 | NCR3LG1 | Natural cytotoxicity triggering receptor 3 ligand 1 |
| Q9NQX7 | ITM2C | Integral membrane protein 2C |
| Q8WVX9 | FAR1 | Fatty acyl-CoA reductase 1 |
| Q9HDC9 | APMAP | Adipocyte plasma membrane-associated protein |
| Q07954 | LRP1 | Prolow-density lipoprotein receptor-related protein 1 |
| O00238 | BMPR1B | Bone morphogenetic protein receptor type-1B |
| P10586 | PTPRF | Receptor-type tyrosine-protein phosphatase F |
| Q13641 | TPBG | Trophoblast glycoprotein |
| Q8TCZ2 | CD99L2 | CD99 antigen-like protein 2 |
| O14786 | NRP1 | Neuropilin-1 |
| Q13740 | ALCAM | CD166 antigen |
| O75976 | CPD | Carboxypeptidase D |
| Q8TCG1 | KIAA1524 | Protein CIP2A |
| Q9Y2A7 | NCKAP1 | Nck-associated protein 1 |
| P23229 | ITGA6 | Integrin alpha-6 |
| P22413 | ENPP1 | Ectonucleotide pyrophosphatase/phosphodiesterase family |
| | | member 1 |
| O76095 | JTB | Protein JTB |
| P15260 | IFNGR1 | Interferon gamma receptor 1 |
| Q86XX4 | FRAS1 | Extracellular matrix protein FRAS1 |
| Q8IV08 | PLD3 | 5′-3′ exonuclease PLD3 |
| Q96L58 | B3GALT6 | Beta-1,3-galactosyltransferase 6 |
| Q9Y287 | ITM2B | Integral membrane protein 2B |
| P49257 | LMAN1 | Protein ERGIC-53 |
| Q9NQS3 | PRR3 | Nectin-3 |
| Q15768 | EFNB3 | Ephrin-B3 |
| Q7Z3J2 | C16orf62 | VPS35 endosomal protein sorting factor-like |
| P34741 | SDC2 | Syndecan-2 |
| Q14789 | GOLGB1 | Golgin subfamily B member 1 |
| Q16832 | TKT | Discoidin domain-containing receptor 2 |
| A4D1S0 | KLRG2 | Killer cell lectin-like receptor subfamily G member 2 |
| P51571 | SSR4 | Translocon-associated protein subunit delta |
| Q5KU26 | CLP1 | Collectin-12 |
| P04843 | RPN1 | Dolichyl-diphosphooligosaccharide-protein |
| | | glycosyltransferase subunit 1 |
| Q6UXM1 | LIG3 | Leucine-rich repeats and immunoglobulin-like domains |
| | | protein 3 |
| Q6ZRP7 | QSOX2 | Sulfhydryl oxidase 2 |
| P17301 | ITGA2 | Integrin alpha-2 |
| O15031 | PLXNB2 | Plexin-B2 |
| O75379 | VAMP4 | Vesicle-associated membrane protein 4 |
| Q92692 | PVRL2 | Nectin-2 |
| Q9Y4D8 | HECTD4 | Probable E3 ubiquitin-protein ligase HECTD4 |
| P43121 | MCAM | Cell surface glycoprotein MUC18 |
| Q5JTH9 | RRP12 | RRP12-like protein |
| P04626 | ERBB2 | Receptor tyrosine-protein kinase erbB-2 |
| Q15738 | NSDHL | Sterol-4-alpha-carboxylate 3-dehydrogenase, decarboxylating |
| O75051 | PLXNA2 | Plexin-A2 |
| Q9HCM2 | PLXNA4 | Plexin-A4 |
| Q9UIW2 | PLXNA1 | Plexin-A1 |
| O60476 | MAN1A2 | Mannosyl-oligosaccharide 1,2-alpha-mannosidase IB |
| A8MVW0 | FAM171A2 | Protein FAM171A2 |
| O60512 | B4GALT3 | Beta-1,4-galactosyltransferase 3 |
| P33908 | MAN1A1 | Mannosyl-oligosaccharide 1,2-alpha-mannosidase IA |
| Q96AJ9 | VTI1A | Vesicle transport through interaction with t-SNAREs homolog |
| | | 1A |
| P18084 | ITGB5 | Integrin beta-5 |
| P19021 | PAM | Peptidyl-glycine alpha-amidating monooxygenase |
| Q7KYR7 | BTN2A1 | Butyrophilin subfamily 2 member A1 |
| Q96IQ7 | CTH | V-set and immunoglobulin domain-containing protein 2 |
| Q9NZ53 | PODXL2 | Podocalyxin-like protein 2 |
| Q10472 | GALNT1 | Polypeptide N-acetylgalactosaminyltransferase 1 |
| P00533 | EGFR | Epidermal growth factor receptor |
| Q9BZF1 | OSBPL8 | Oxysterol-binding protein-related protein 8 |
| P67812 | SEC11A | Signal peptidase complex catalytic subunit |

TABLE 3
Examples of multi pass (polytopic) transmembrane proteins that
can be used scaffold proteins with accessible N- or C-termini

| UniProt | | |
|---|---|---|
| Entry | Gene | Protein names |
| P32418 | SLC8A1 | Sodium/calcium exchanger 1 |
| Q96QD8 | SLC38A2 | Sodium-coupled neutral amino acid transporter |
| | | 2 |
| O00155 | GPR25 | Probable G-protein coupled receptor 25 |
| Q92839 | HAS1 | Hyaluronan synthase 1 |
| Q9C0H2 | TTYH3 | Protein tweety homolog 3 |
| Q6RW13 | AGTRAP | Type-1 angiotensin II receptor-associated |
| | | protein |
| O15431 | SLC31A1 | High affinity copper uptake protein 1 |
| Q96EC8 | YIPF6 | Protein YIPF6 |
| Q99808 | SLC29A1 | Equilibrative nucleoside transporter 1 |
| Q99805 | TM9SF2 | Transmembrane 9 superfamily member 2 |
| O95477 | ABCA1 | Phospholipid-transporting ATPase ABCA1 |
| Q8NE79 | POP1 | Blood vessel epicardial substance |
| Q08722 | CD47 | Leukocyte surface antigen CD47 |
| Q92544 | TM9SF4 | Transmembrane 9 superfamily member 4 |
| P41440 | FLOT1 | Reduced folate transporter |
| Q9BVC6 | TMEM109 | Transmembrane protein 109 |
| Q13433 | SLC39A6 | Zinc transporter ZIP6 |
| Q15043 | SLC39A14 | Zinc transporter ZIP14 |
| Q9BSA4 | TTYH2 | Protein tweety homolog 2 |
| P43007 | SLC1A4 | Neutral amino acid transporter A |
| O95490 | LPHN2 | Adhesion G protein-coupled receptor L2 |
| Q9NZH0 | GPRC5B | G-protein coupled receptor family C group 5 |
| | | member B |
| Q13530 | SERINC3 | Serine incorporator 3 |
| P53794 | SLC5A3 | Sodium/myo-inositol cotransporter |
| Q9Y548 | YIPF1 | YIP1 family member 1 |
| Q8NBN3 | TMEM87A | Transmembrane protein 87A |
| Q08357 | SLC20A2 | Sodium-dependent phosphate transporter 2 |
| O75908 | ACAT2 | Sterol O-acyltransferase 2 |
| Q8TCT7 | IMP4 | Signal peptide peptidase-like 2B |

TABLE 4
Examples of multi-pass (polytopic) transmembrane proteins that can
contain transmembrane domains that be used scaffold proteins

| UniProt | | |
|---|---|---|
| Entry | Gene | Protein name |
| P60033 | CD81 | CD81 antigen |
| P21926 | CD9 | CD9 antigen |
| O43657 | TSPAN6 | Tetraspanin-6 |
| O14828 | SCAMP3 | Secretory carrier-associated membrane protein 3 |
| Q15758 | SLC1A5 | Neutral amino acid transporter B |
| O15440 | ABCC5 | Multidrug resistance-associated protein 5 |
| O15127 | SCAMP2 | Secretory carrier-associated membrane protein 2 |
| P05023 | ATP1A1 | Sodium/potassium-transporting ATPase subunit alpha-1 |
| Q969E2 | SCAMP4 | Secretory carrier-associated membrane protein 4 |
| P50993 | ATP1A2 | Sodium/potassium-transporting ATPase subunit alpha-2 |
| P13637 | ATP1A3 | Sodium/potassium-transporting ATPase subunit alpha-3 |
| O75954 | TSPAN9 | Tetraspanin-9 |
| P53985 | SLC16A1 | Monocarboxylate transporter |
| P08962 | CD63 | CD63 antigen |
| P23942 | PRPH | Peripherin-2 |
| O43759 | SYNGR1 | Synaptogyrin-1 |
| Q93050 | ATP6V0A1 | V-type proton ATPase 116 kDa subunit a isoform 1 |
| P30825 | SLC7A1 | High affinity cationic amino acid transporter 1 |
| P17302 | GJA1 | Gap junction alpha-1 protein |
| O43760 | SYNGR2 | Synaptogyrin-2 |
| O60637 | TSPAN3 | Tetraspanin-3 |
| O15126 | SCAMP1 | Secretory carrier-associated membrane protein 1 |
| P28328 | PAF1 | Peroxisome biogenesis factor 2 |
| P41732 | TSPAN7 | Tetraspanin-7 |
| Q9P003 | CNIH4 | Protein cornichon homolog 4 |
| O14817 | TSPAN4 | Tetraspanin-4 |
| Q9NRX5 | SERINC1 | Serine incorporator 1 |
| Q5T4S7 | UBR4 | E3 ubiquitin-protein ligase UBR4 |
| Q01650 | SLC7A5 | Large neutral amino acids transporter small subunit 1 |
| Q13491 | GPM6B | Neuronal membrane glycoprotein M6-b |
| Q8NG11 | TSPAN14 | Tetraspanin-14 |
| Q8N144 | GJC1 | Gap junction delta-3 protein |
| P36383 | GJC1 | Gap junction gamma-1 protein |
| Q9NQC3 | RTN4 | Reticulon-4 |
| Q8IWA5 | SLC44A2 | Choline transporter-like protein 2 |
| Q92536 | SLC7A6 | Y + L amino acid transporter 2 |
| P48509 | CD151 | CD151 antigen |
| P51674 | GPM6A | Neuronal membrane glycoprotein M6-a |
| P08247 | SYP | Synaptophysin |
| Q9BVI4 | NOC4L | Nucleolar complex protein 4 homolog |
| P11166 | SLC2A1 | Solute carrier family 2, facilitated glucose transporter member 1 |
| Q8WTV0 | SCARB1 | Scavenger receptor class B member 1 |
| O15321 | TM9SF1 | Transmembrane 9 superfamily member 1 |
| Q969M3 | YIPF5 | Protein YIPF5 |
| O60831 | PRAF2 | PRA1 family protein 2 |
| Q8WWI5 | SLC44A1 | Choline transporter-like protein 1 |
| Q7Z3C6 | ATG9A | Autophagy-related protein 9A |
| Q16625 | OCLN | Occludin |
| O15258 | RER1 | Protein RER1 |
| Q12893 | TMEM115 | Transmembrane protein 115 |
| P62079 | TSPAN5 | Tetraspanin-5 |
| Q9BSR8 | YIPF4 | Protein YIPF4 |
| P43003 | SLC1A3 | Excitatory amino acid transporter 1 |
| P54219 | VAT1 | Chromaffin granule amine transporter |
| P05141 | SLC25A5 | ADP/ATP translocase 2 |
| Q658P3 | STEAP3 | Metalloreductase STEAP3 |
| Q9H813 | TMEM206 | Proton-activated chloride channel |
| O75915 | ARL6IP5 | PRA1 family protein 3 |
| Q9Y6M5 | SLC30A1 | Zinc transporter 1 |
| Q86T03 | TMEM55B | Type 1 phosphatidylinositol 4,5-bisphosphate 4-phosphatase |
| Q9Y5Y0 | FLVCR1 | Feline leukemia virus subgroup C receptor-related protein 1 |
| P55011 | SLC12A2 | Solute carrier family 12 member 2 |
| Q8NCG7 | DAGLB | Sn1-specific diacylglycerol lipase beta |
| P20020 | ATP2B1 | Plasma membrane calcium-transporting ATPase 1 |
| P11169 | SLC2A3 | Solute carrier family 2, facilitated glucose transporter member 3 |
| Q9HD45 | TM9SF3 | Transmembrane 9 superfamily member 3 |
| Q8NE01 | CNNM3 | Metal transporter CNNM3 |
| Q9H2V7 | SPNS1 | Protein spinster homolog 1 |
| P23634 | ATP2B4 | Plasma membrane calcium-transporting ATPase 4 |
| Q9BXP2 | SLC12A9 | Solute carrier family 12 member 9 |
| Q3ZAQ7 | VMA21 | Vacuolar ATPase assembly integral membrane protein VMA21 |
| Q96RQ1 | ERGIC2 | ER-Golgi intermediate compartment protein 2 |
| Q9Y487 | ATP6V0A2 | V-type proton ATPase 116 kDa subunit a isoform 2 |
| Q9H3U5 | MFSD1 | Major facilitator superfamily domain-containing protein 1 |
| Q53GQ0 | HSD17B12 | Very-long-chain 3-oxoacyl-CoA reductase |
| Q9C0B5 | ZDHHC5 | Palmitoyltransferase ZDHHC5 |
| Q9BSJ8 | ESYT1 | Extended synaptotagmin-1 |
| Q8IZA0 | KIAA0319L | Dyslexia-associated protein KIAA0319-like protein |
| P12236 | SLC25A6 | ADP/ATP translocase 3 |
| Q86VR2 | FAM134C | Reticulophagy regulator 3 |
| Q9UP95 | SLC12A4 | Solute carrier family 12 member 4 |
| Q9UL54 | TAOK2 | Serine/threonine-protein kinase TAO2 |
| P51798 | CLCN7 | Chloride channel 7 alpha subunit |
| Q16739 | UGCG | Ceramide glucosyltransferase |
| Q6P9B9 | INTS5 | Integrator complex subunit 5 |
| Q8WUM9 | SLC20A1 | Sodium-dependent phosphate transporter 1 |
| Q9NTJ5 | SACM1L | Phosphatidylinositide phosphatase SAC1 |
| Q8NER1 | TRPV1 | Trans. receptor potential cation channel subfamily V member 1 |
| Q9UPY5 | SLC7A11 | Cystine/glutamate transporter |
| Q8NB49 | ATP11C | Phospholipid-transporting ATPase IG |

Further non-limiting examples of other scaffold proteins comprising a transmembrane domain that can be used with the present disclosure include: aminopeptidase N (CD 13); Neprilysin, AKA membrane metalloendopeptidase (MME); ectonucleotide pyrophosphatase/phosphodiesterase family member 1 (ENPP1); Neuropilin-1 (NRP1); PDGFR, GPI anchor proteins, lactadherin, LAMP2, and LAMP2B)

In some embodiments, the transmembrane domain comprises an amino acid sequence that is at least 70%, at least 75%, at least 80%, at least 85%, at least 90%, at least 95%, at least 96%, at least 97%, at least 98%, at least 99%, or 100% identical to the transmembrane domain of a wild-type ephrin receptor (e.g., an ephrin receptor TM domain comprising an amino acid sequence selected from the group consisting of SEQ TD NOs: 35-48). In some embodiments, the transmembrane domain of the polypeptide is the transmembrane domain of a wild-type ephrin receptor (e.g., an ephrin receptor TM domain comprising an amino acid sequence selected from the group consisting of SEQ TD NOs: 35-48).

In some embodiments, the transmembrane domain comprises an amino acid sequence that is at least 70%, at least 75%, at least 8%%, at least 85%, at least 90%, at least 95%, at least 96%, at least 97%, at least 98%, at least 99%, or 100% identical to the transmembrane domain of a wild-type FPRP (e.g., an FPRP TM domain comprising an amino acid sequence of SEQ TD NO: 49). In some embodiments, the transmembrane domain of the polypeptide is the transmembrane domain of a wild-type FPRP (e.g., an FPRP TM domain comprising an amino acid sequence of SEQ ID NO: 49).

TABLE 5
Non-limiting examples of TM domains.

| | Protein | | |
|---|---|---|---|
| SEQ | (UniProt ID | | |
| ID: | No.) | Region | Sequence |
| 35 | EPHA1 (P21709) | 548-568 | IVAVIFGLLLGAALLLGILVF |
| 36 | EPHA2 (P29317) | 538-558 | IGGVAVGVVLLLVLAGVGFFI |
| 37 | EPHA3 (P29320) | 542-565 | VVMIAISAAVAIILLTVVIYVL |
| | | | IG |
| 38 | EPHA4 (P54764) | 548-569 | VLLVSVSGSVVLVVILIAAFVI |
| 39 | EPHA5 (P54756) | 574-594 | VIAVSVTVGVILLAVVIGVLL |
| 40 | EPHA6 (Q9UF33) | 551-571 | IATAAVGGFTLLVILTLFFLI |
| 41 | EPHA7 (Q15375) | 556-576 | IIIAVVAVAGTIILVFMVFGF |
| 42 | EPHA8 (P29322) | 543-563 | IVWICLTLITGLVVLLLLLIC |
| 43 | EPHA10 (Q5JZY3) | 566-586 | IVVTVVTISALLVLGSVMSVL |
| 44 | EPHB1 (P54762) | 541-563 | LIAGSAAAGVVFVVSLVAISIVC |
| 45 | EPHB2 (P29323) | 544-564 | IIGSSAAGLVFLIAVVVIAIV |
| 46 | EPHB3 (P54753) | 560-580 | IVGSATAGLVFVVAVVVIAIV |
| 47 | EPHB4 (P54760) | 540-560 | LIAGTAVVGVVLVLVVIVVAV |
| 48 | EPHB6 (O15197) | 595-615 | LVIGSILGALAFLLLAAITVL |
| 49 | FPRP | 833-853 | LLIGVGLSTVIGLLSCLIGYC |

In some embodiments, the transmembrane domain comprises the amino acid sequence of the transmembrane domain of a wild-type ephrin receptor (e.g., an ephrin receptor transmembrane domain comprising an amino acid sequence selected from the group consisting of SEQ ID NOs: 35-48) except one amino acid mutation, two amino acid mutations, three amino acid mutations, four amino acid mutations, five amino acid mutations, six amino acid mutations, seven amino acid mutations, or more than seven amino acid mutations. The mutation(s) can be substitution(s), insertion(s), deletion(s), or any combination thereof.

In some embodiments, the transmembrane domain comprises the amino acid sequence of the transmembrane domain of a wild-type FPRP (e.g., an FPRP transmembrane domain comprising an amino acid sequence of SEQ ID NO: 50) except one amino acid mutation, two amino acid mutations, three amino acid mutations, four amino acid mutations, five amino acid mutations, six amino acid mutations, seven amino acid mutations, or more than seven amino acid mutations. The mutation(s) can be substitution(s), insertion(s), deletion(s), or any combination thereof.

In some embodiments, the FcRn binding polypeptide comprises a transmembrane domain homo-domain dimerization motif which increases interaction between two or more of the polypeptides at the transmembrane domain. In certain embodiments, the transmembrane domain homo-domain dimer motif is a transmembrane leucine zipper motif. In certain embodiments, the transmembrane domain homo-dimer motif is a transmembrane glycine zipper motif. Methods to modify and assay transmembrane domain dimerization are known in the art, see, e.g., Bocharov et al. J Biol Chem. 2008 Oct. 24; 283(43):29385-95.

In some embodiments, the transmembrane domain comprises the amino acid sequence of the transmembrane domain of a wild-type ephrin receptor (e.g., an ephrin receptor transmembrane domain comprising an amino acid sequence selected from the group consisting of SEQ ID NOs: 35-48) and its length is 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NOs: 35-48.

In some embodiments, the transmembrane domain comprises the amino acid sequence of the transmembrane domain of a wild-type FPRP (e.g., an FPRP transmembrane domain comprising an amino acid sequence of SEQ ID NO:50) and its length is 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO:50.

### (b) Selecting Transmembrane Scaffold Proteins

Several factors can be considered in selecting a scaffold protein for use as a transmembrane domain in a FcRn binding polypeptide of the disclosure. Among these factors are a recognition of what the particular transmembrane protein type (Type I, II, or polytopic) is of the protein being considered for use as the source of the transmembrane domain, a recognition of the natural subcellular location of the transmembrane protein, and a recognition that the FcRn binding polypeptide and the transmembrane domain in a fusion protein according to the disclosure, may affect each other's function in the overall process described herein for display of the FcRn binding polypeptide on the surface of nanovesicles.

As noted above, the four types of scaffold proteins can be distinguished from one another by the relative orientation of the N- and C-termini with respect to the cytoplasm and the endoplasmic reticulum or the nanovesicle (e.g. an EV) lumen and whether the transmembrane region of the protein traverses a nanovesicle (e.g. an EV) membrane only once (“single pass” transmembrane region) or comprises two or more membrane-spanning regions so that the protein as a whole passes through a membrane more than once (multi-pass transmembrane region).

Knowing that a transmembrane region is derived from a particular type of transmembrane protein suggests a preferred orientation and location for the transmembrane domain relative to the FcRn-binding site in a FcRn binding polypeptide of the disclosure. This is particularly important with respect to Type I and Type II transmembrane proteins, which have fixed orientations and locations for their N- and C-termini with respect to the cytoplasm and nanovesicle (e.g. an EV) lumen on either side of the transmembrane region. For example, when a transmembrane region from a Type I transmembrane protein is used as the scaffold protein (referred to as a T1 scaffold) of a FcRn binding polypeptide of the disclosure, the FcRn binding site is N-terminal to the transmembrane domain (as depicted in FIG. 2). Thus, the most common configurations of a FcRn binding polypeptide of the present disclosure that have a Type I-derived transmembrane domain will comprise an N-terminal to C-terminal linear structure illustrated as follows:

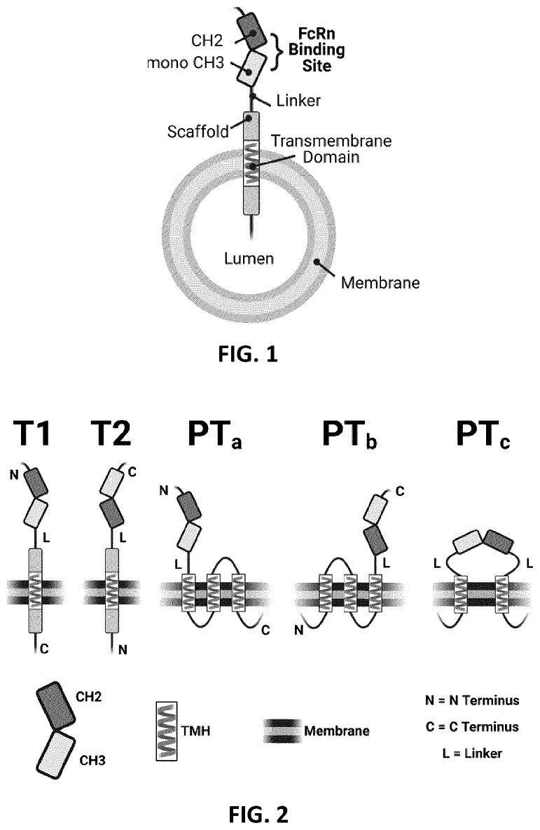

(FcRn binding site)-L-(T1 scaffold), (1)

where the L in the formulae represents a direct peptide bond linking two domains or a linker sequence of one or more amino acid residues. See FIG. 1 for a schematic of a nanovesicle comprising an FcRn binding polypeptide that contains a type I transmembrane domain.

In addition, a FcRn binding polypeptide comprising a Type I-derived transmembrane domain preferably comprises an N-terminal signal sequence (e.g. signal peptide), which is characteristic of Type I transmembrane proteins to direct the N-terminus of the fusion protein through the ER membrane and into the ER lumen.

In specific embodiments, the FcRn binding polypeptide is linked to a T1 scaffold protein that comprises an ectodomain and a transmembrane domain that are derived from EphA1. In some embodiments, the FcRn binding polypeptide comprises an amino acid sequence identical or similar to the entire ectodomain and transmembrane domain region of wild-type EphA1 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA1, and wherein said polypeptide exhibits decreased or no binding to ephrins as compared to the parental Eph receptor. In some embodiments, the polypeptide comprises an amino acid sequence identical or similar to SEQ ID No:50 or a fragment thereof and has at least 70%, at least 80%, at least 85%, at least 90%, at least 95%, at least 96%, at least 97%, at least 98%, or at least 99% sequence identity to SEQ ID No: 50, and wherein said polypeptide exhibits decreased or no binding to ephrins as compared to the parental Eph receptor. In some embodiments, the portion of the polypeptide derived from EphA1 is fused to one or more heterologous proteins.

In specific embodiments, the FcRn binding polypeptide is linked to a T1 scaffold protein that comprises an ectodomain and a transmembrane domain that are derived from EphA2. In some embodiments, the FcRn binding polypeptide comprises an amino acid sequence identical or similar to the entire ectodomain and transmembrane domain region of wild-type EphA2 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA2, and wherein said polypeptide exhibits decreased or no binding to ephrins as compared to the parental Eph receptor. In some embodiments, the polypeptide comprises an amino acid sequence identical or similar to SEQ ID No: 51 or a fragment thereof and has at least 70%, at least 80%, at least 85%, at least 90%, at least 95%, at least 96%, at least 97%, at least 98%, or at least 99% sequence identity to SEQ ID No: 51, and wherein said polypeptide exhibits decreased or no binding to ephrins as compared to the parental Eph receptor. In some embodiments, the portion of the polypeptide derived from EphA2 is fused to one or more heterologous proteins.

In specific embodiments, the FcRn binding polypeptide is linked to a T1 scaffold protein that comprises an ectodomain and a transmembrane domain that are derived from EphA3. In some embodiments, the FcRn binding polypeptide comprises an amino acid sequence identical or similar to the entire ectodomain and transmembrane domain region of wild-type EphA3 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA3, and wherein said polypeptide exhibits decreased or no binding to ephrins as compared to the parental Eph receptor. In some embodiments, the polypeptide comprises an amino acid sequence identical or similar to SEQ ID No: 52 or a fragment thereof and has at least 70%, at least 80%, at least 85%, at least 90%, at least 95%, at least 96%, at least 97%, at least 98%, or at least 99% sequence identity to SEQ ID No: 52, and wherein said polypeptide exhibits decreased or no binding to ephrins as compared to the parental Eph receptor. In some embodiments, the portion of the polypeptide derived from EphA3 is fused to one or more heterologous proteins.

In specific embodiments, the FcRn binding polypeptide is linked to a T1 scaffold protein that comprises an ectodomain and a transmembrane domain that are derived from EphA4. In some embodiments, the FcRn binding polypeptide comprises an amino acid sequence identical or similar to the entire ectodomain and transmembrane domain region of wild-type EphA4 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA4, and wherein said polypeptide exhibits decreased or no binding to ephrins as compared to the parental Eph receptor. In some embodiments, the polypeptide comprises an amino acid sequence identical or similar to SEQ ID No: 53 or a fragment thereof and has at least 70%, at least 80%, at least 85%, at least 90%, at least 95%, at least 96%, at least 97%, at least 98%, or at least 99% sequence identity to SEQ ID No: 53, and wherein said polypeptide exhibits decreased or no binding to ephrins as compared to the parental Eph receptor. In some embodiments, the portion of the polypeptide derived from EphA4 is fused to one or more heterologous proteins.

In specific embodiments, the FcRn binding polypeptide is linked to a T1 scaffold protein that comprises an ectodomain and a transmembrane domain that are derived from EphA5. In some embodiments, the FcRn binding polypeptide comprises an amino acid sequence identical or similar to the entire ectodomain and transmembrane domain region of wild-type EphA5 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA5, and wherein said polypeptide exhibits decreased or no binding to ephrins as compared to the parental Eph receptor. In some embodiments, the polypeptide comprises an amino acid sequence identical or similar to SEQ ID No: 54 or a fragment thereof and has at least 70%, at least 80%, at least 85%, at least 90%, at least 95%, at least 96%, at least 97%, at least 98%, or at least 99% sequence identity to SEQ ID No: 54, and wherein said polypeptide exhibits decreased or no binding to ephrins as compared to the parental Eph receptor. In some embodiments, the portion of the polypeptide derived from EphA5 is fused to one or more heterologous proteins.

In specific embodiments, the FcRn binding polypeptide is linked to a T1 scaffold protein that comprises an ectodomain and a transmembrane domain that are derived from EphA6. In some embodiments, the FcRn binding polypeptide comprises an amino acid sequence identical or similar to the entire ectodomain and transmembrane domain region of wild-type EphA6 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA6, and wherein said polypeptide exhibits decreased or no binding to ephrins as compared to the parental Eph receptor. In some embodiments, the polypeptide comprises an amino acid sequence identical or similar to SEQ ID No: 55 or a fragment thereof and has at least 70%, at least 80%, at least 85%, at least 90%, at least 95%, at least 96%, at least 97%, at least 98%, or at least 99% sequence identity to SEQ ID No: 55, and wherein said polypeptide exhibits decreased or no binding to ephrins as compared to the parental Eph receptor. In some embodiments, the portion of the polypeptide derived from EphA6 is fused to one or more heterologous proteins.

In specific embodiments, the FcRn binding polypeptide is linked to a T1 scaffold protein that comprises an ectodomain and a transmembrane domain that are derived from EphA7. In some embodiments, the FcRn binding polypeptide comprises an amino acid sequence identical or similar to the entire ectodomain and transmembrane domain region of wild-type EphA7 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA7, and wherein said polypeptide exhibits decreased or no binding to ephrins as compared to the parental Eph receptor. In some embodiments, the polypeptide comprises an amino acid sequence identical or similar to SEQ ID No: 56 or a fragment thereof and has at least 70%, at least 80%, at least 85%, at least 90%, at least 95%, at least 96%, at least 97%, at least 98%, or at least 99% sequence identity to SEQ ID No: 56, and wherein said polypeptide exhibits decreased or no binding to ephrins as compared to the parental Eph receptor. In some embodiments, the portion of the polypeptide derived from EphA7 is fused to one or more heterologous proteins.

In specific embodiments, the FcRn binding polypeptide is linked to a T1 scaffold protein that comprises an ectodomain and a transmembrane domain that are derived from EphA8. In some embodiments, the FcRn binding polypeptide comprises an amino acid sequence identical or similar to the entire ectodomain and transmembrane domain region of wild-type EphA8 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA8, and wherein said polypeptide exhibits decreased or no binding to ephrins as compared to the parental Eph receptor. In some embodiments, the polypeptide comprises an amino acid sequence identical or similar to SEQ ID No: 57 or a fragment thereof and has at least 70%, at least 80%, at least 85%, at least 90%, at least 95%, at least 96%, at least 97%, at least 98%, or at least 99% sequence identity to SEQ ID No: 57, and wherein said polypeptide exhibits decreased or no binding to ephrins as compared to the parental Eph receptor. In some embodiments, the portion of the polypeptide derived from EphA8 is fused to one or more heterologous proteins.

In specific embodiments, the FcRn binding polypeptide is linked to a T1 scaffold protein that comprises an ectodomain and a transmembrane domain that are derived from EphA10. In some embodiments, the FcRn binding polypeptide comprises an amino acid sequence identical or similar to the entire ectodomain and transmembrane domain region of wild-type EphA10 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphA10, and wherein said polypeptide exhibits decreased or no binding to ephrins as compared to the parental Eph receptor. In some embodiments, the polypeptide comprises an amino acid sequence identical or similar to SEQ ID No: 58 or a fragment thereof and has at least 70%, at least 80%, at least 85%, at least 90%, at least 95%, at least 96%, at least 97%, at least 98%, or at least 99% sequence identity to SEQ ID No: 58, and wherein said polypeptide exhibits decreased or no binding to ephrins as compared to the parental Eph receptor. In some embodiments, the portion of the polypeptide derived from EphA10 is fused to one or more heterologous proteins.

In specific embodiments, the FcRn binding polypeptide is linked to a T1 scaffold protein that comprises an ectodomain and a transmembrane domain that are derived from EphB1. In some embodiments, the FcRn binding polypeptide comprises an amino acid sequence identical or similar to the entire ectodomain and transmembrane domain region of wild-type EphB1 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphB1, and wherein said polypeptide exhibits decreased or no binding to ephrins as compared to the parental Eph receptor. In some embodiments, the polypeptide comprises an amino acid sequence identical or similar to SEQ ID No: 59 or a fragment thereof and has at least 70%, at least 80%, at least 85%, at least 90%, at least 95%, at least 96%, at least 97%, at least 98%, or at least 99% sequence identity to SEQ ID No: 59, and wherein said polypeptide exhibits decreased or no binding to ephrins as compared to the parental Eph receptor. In some embodiments, the portion of the polypeptide derived from EphB1 is fused to one or more heterologous proteins.

In specific embodiments, the FcRn binding polypeptide is linked to a T1 scaffold protein that comprises an ectodomain and a transmembrane domain that are derived from EphB2. In some embodiments, the FcRn binding polypeptide comprises an amino acid sequence identical or similar to the entire ectodomain and transmembrane domain region of wild-type EphB2 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphB2, and wherein said polypeptide exhibits decreased or no binding to ephrins as compared to the parental Eph receptor. In some embodiments, the polypeptide comprises an amino acid sequence identical or similar to SEQ ID No: 60 or a fragment thereof and has at least 70%, at least 80%, at least 85%, at least 90%, at least 95%, at least 96%, at least 97%, at least 98%, or at least 99% sequence identity to SEQ ID No: 60, and wherein said polypeptide exhibits decreased or no binding to ephrins as compared to the parental Eph receptor. In some embodiments, the portion of the polypeptide derived from EphB2 is fused to one or more heterologous proteins.

In specific embodiments, the FcRn binding polypeptide is linked to a T1 scaffold protein that comprises an ectodomain and a transmembrane domain that are derived from EphB3. In some embodiments, the FcRn binding polypeptide comprises an amino acid sequence identical or similar to the entire ectodomain and transmembrane domain region of wild-type EphB3 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphB3, and wherein said polypeptide exhibits decreased or no binding to ephrins as compared to the parental Eph receptor. In some embodiments, the polypeptide comprises an amino acid sequence identical or similar to SEQ ID No: 61 or a fragment thereof and has at least 70%, at least 80%, at least 85%, at least 90%, at least 95%, at least 96%, at least 97%, at least 98%, or at least 99% sequence identity to SEQ ID No: 61, and wherein said polypeptide exhibits decreased or no binding to ephrins as compared to the parental Eph receptor. In some embodiments, the portion of the polypeptide derived from EphB3 is fused to one or more heterologous proteins

In specific embodiments, the FcRn binding polypeptide is linked to a T1 scaffold protein that comprises an ectodomain and a transmembrane domain that are derived from EphB4. In some embodiments, the FcRn binding polypeptide comprises an amino acid sequence identical or similar to the entire ectodomain and transmembrane domain region of wild-type EphB4 or a fragment thereof and has at least 70%, at least 80%, at least 85%, at least 90%, at least 95%, at least 96%, at least 97%, at least 98%, or at least 99% sequence identity to the amino acid sequence of the entire ectodomain and transmembrane domain region of wild-type EphB4, and wherein said polypeptide exhibits decreased or no binding to ephrins as compared to the parental Eph receptor. In some embodiments, the polypeptide comprises an amino acid sequence identical or similar to SEQ ID No: 62 or a fragment thereof and has at least 70%, at least 80%, at least 85%, at least 90%, at least 95%, at least 96%, at least 97%, at least 98%, or at least 99% sequence identity to SEQ ID No: 62, and wherein said polypeptide exhibits decreased or no binding to ephrins as compared to the parental Eph receptor. In some embodiments, the portion of the polypeptide derived from EphB4 is fused to one or more heterologous proteins.

In specific embodiments, the FcRn binding polypeptide is linked to a T1 scaffold protein that comprises an ectodomain and a transmembrane domain that are derived from EphB6. In some embodiments, the FcRn binding polypeptide comprises an amino acid sequence identical or similar to the entire ectodomain and transmembrane domain region of wild-type EphB36 or a fragment thereof and has at least 70%, at least 80%, at least 85%, at least 90%, at least 95% at least 96%, at least 97% at least 98%, or at least 99% sequence identity to the amino acid sequence of the entire ectodomain and transmembrane domain region of wild-type EphB6, and wherein said polypeptide exhibits decreased or no binding to ephrins as compared to the parental Eph receptor. In some embodiments, the polypeptide comprises an amino acid sequence identical or similar to SEQ TD No: 63 or a fragment thereof and has at least 70%, at least 80%, at least 85%, at least 90%, at least 95%, at least 96%, at least 97% at least 98%, or at least 99% sequence identity to SEQ TD No: 63, and wherein said polypeptide exhibits decreased or no binding to ephrins as compared to the parental Eph receptor. In some embodiments, the portion of the polypeptide derived from EphB36 is fused to one or more heterologous proteins.

TABLE 6
Exemplary Eph receptor derived scaffold proteins comprising
ectodomain and transmembrane domain).

| SEQ | | |
|---|---|---|
| ID: | Protein | Sequence |
| 50 | EPHA1 | EVTLMDTSKAQGELGWLLDPPKDGWSEQQQILNGTPLYMYQDCPMQGRRDTDHW |
| | (27-596) | LRSNWIYRGEEASRVHVELQFTVRDCKSFPGGAGPLGCKETFNLLYMESDQDVGIQLR |
| | | RPLFQKVTTVAADQSFTIRDLVSGSVKLNVERCSLGRLTRRGLYLAFHNPGACVALVSV |
| | | RVFYQRCPETLNGLAQFPDTLPGPAGLVEVAGTCLPHARASPRPSGAPRMHCSPDGE |
| | | WLVPVGRCHCEPGYEEGGSGEACVACPSGSYRMDMDTPHCLTCPQQSTAESEGATI |
| | | CTCESGHYRAPGEGPQVACTGPPSAPRNLSFSASGTQLSLRWEPPADTGGRQDVRYS |
| | | VRCSQCQGTAQDGGPCQPCGVGVHFSPGARGLTTPAVHVNGLEPYANYTFNVEAQ |
| | | NGVSGLGSSGHASTSVSISMGHAESLSGLSLRLVKKEPRQLELTWAGSRPRSPGANLTY |
| | | ELHVLNQDEERYQMVLEPRVLLTELQPDTTYIVRVRMLTPLGPGPFSPDHEFRTSPPVS |
| | | RGLTGGEIVAVIFGLLLGAALLLGILVFRSRRAQRQRQQRQRDRATDVDREDKLWL |
| 51 | EPHA2 | EVVLLDFAAAGGELGWLTHPYGKGWDLMQNIMNDMPIYMYSVCNVMSGDQDNW |
| | (28-585) | LRTNWVYRGEAERIFIELKFTVRDCNSFPGGASSCKETFNLYYAESDLDYGTNFQKRLFT |
| | | KIDTIAPDEITVSSDFEARHVKLNVEERSVGPLTRKGFYLAFQDIGACVALLSVRVYYKKC |
| | | PELLQGLAHFPETIAGSDAPSLATVAGTCVDHAVVPPGGEEPRMHCAVDGEWLVPIG |
| | | QCLCQAGYEKVEDACQACSPGFFKFEASESPCLECPEHTLPSPEGATSCECEEGFFRAP |
| | | QDPASMPCTRPPSAPHYLTAVGMGAKVELRWTPPQDSGGREDIVYSVTCEQCWPES |
| | | GECGPCEASVRYSEPPHGLTRTSVTVSDLEPHMNYTFTVEARNGVSGLVTSRSFRTASV |
| | | SINQTEPPKVRLEGRSTTSLSVSWSIPPPQQSRVWKYEVTYRKKGDSNSYNVRRTEGFS |
| | | VTLDDLAPDTTYLVQVQALTQEGQGAGSKVHEFQTLSPEGSGNLAVIGGVAVGVVLLL |
| | | VLAGVGFFIHRRRKNQRARQSPEDVYFSKSEQLKPL |
| 52 | EPHA3 | EVNLLDSKTIQGELGWISYPSHGWEEISGVDEHYTPIRTYQVCNVMDHSQNNWLRTN |
| | (29-590) | WVPRNSAQKIYVELKFTLRDCNSIPLVLGTCKETFNLYYMESDDDHGVKFREHQFTKID |
| | | TIAADESFTQMDLGDRILKLNTEIREVGPVNKKGFYLAFQDVGACVALVSVRVYFKKCP |
| | | FTVKNLAMFPDTVPMDSQSLVEVRGSCVNNSKEEDPPRMYCSTEGEWLVPIGKCSCN |
| | | AGYEERGFMCQACRPGFYKALDGNMKCAKCPPHSSTQEDGSMNCRCENNYFRADK |
| | | DPPSMACTRPPSSPRNVISNINETSVILDWSWPLDTGGRKDVTFNIICKKCGWNIKQC |
| | | EPCSPNVRFLPRQFGLTNTTVTVTDLLAHTNYTFEIDAVNGVSELSSPPRQFAAVSITTN |
| | | QAAPSPVLTIKKDRTSRNSISLSWQEPEHPNGIILDYEVKYYEKQEQETSYTILRARGTNV |
| | | TISSLKPDTIYVFQIRARTAAGYGTNSRKFEFETSPDSFSISGESSQVVMIAISAAVAIILLT |
| | | VVIYVLIGRFCGYKSKHGADEKRLHFGNGHLKL |
| 53 | EPHA4 | EVTLLDSRSVQGELGWIASPLEGGWEEVSIMDEKNTPIRTYQVCNVMEPSQNNWLRT |
| | (30-590) | DWITREGAQRVYIEIKFTLRDCNSLPGVMGTCKETFNLYYYESDNDKERFIRENQFVKID |
| | | TIAADESFTQVDIGDRIMKLNTEIRDVGPLSKKGFYLAFQDVGACIALVSVRVFYKKCPL |
| | | TVRNLAQFPDTITGADTSSLVEVRGSCVNNSEEKDVPKMYCGADGEWLVPIGNCLCN |
| | | AGHEERSGECQACKIGYYKALSTDATCAKCPPHSYSVWEGATSCTCDRGFFRADNDA |
| | | ASMPCTRPPSAPLNLISNVNETSVNLEWSSPQNTGGRQDISYNVVCKKCGAGDPSKC |
| | | RPCGSGVHYTPQQNGLKTTKVSITDLLAHTNYTFEIWAVNGVSKYNPNPDQSVSVTVT |
| | | TNQAAPSSIALVQAKEVTRYSVALAWLEPDRPNGVILEYEVKYYEKDQNERSYRIVRTA |
| | | ARNTDIKGLNPLTSYVFHVRARTAAGYGDFSEPLEVTTNTVPSRIIGDGANSTVLLVSVS |
| | | GSVVLVVILIAAFVISRRRSKYSKAKQEADEEKHLN |
| 54 | EPHA5 | EVNLLDSRTVMGDLGWIAFPKNGWEEIGEVDENYAPIHTYQVCKVMEQNQNNWLL |
| | (60-619) | TSWISNEGASRIFIELKFTLRDCNSLPGGLGTCKETFNMYYFESDDQNGRNIKENQYIKI |
| | | DTIAADESFTELDLGDRVMKLNTEVRDVGPLSKKGFYLAFQDVGACIALVSVRVYYKKC |
| | | PSVVRHLAVFPDTITGADSSQLLEVSGSCVNHSVTDEPPKMHCSAEGEWLVPIGKCMC |
| | | KAGYEEKNGTCQVCRPGFFKASPHIQSCGKCPPHSYTHEEASTSCVCEKDYFRRESDPP |
| | | TMACTRPPSAPRNAISNVNETSVFLEWIPPADTGGRKDVSYYIACKKCNSHAGVCEEC |
| | | GGHVRYLPRQSGLKNTSVMMVDLLAHTNYTFEIEAVNGVSDLSPGARQYVSVNVTTN |
| | | QAAPSPVTNVKKGKIAKNSISLSWQEPDRPNGIILEYEIKYFEKDQETSYTIIKSKETTITAE |
| | | GLKPASVYVFQIRARTAAGYGVFSRRFEFETTPVFAASSDQSQIPVIAVSVTVGVILLAV |
| | | VIGVLLSGSCCECGCGRASSLCAVAHPSLIW |
| 55 | EPHA6 | QVVLLDTTTVLGELGWKTYPLNGWDAITEMDEHNRPIHTYQVCNVMEPNQNNWLR |
| | (34-589) | TNWISRDAAQKIYVEMKFTLRDCNSIPWVLGTCKETFNLFYMESDESHGIKFKPNQYT |
| | | KIDTIAADESFTQMDLGDRILKLNTEIREVGPIERKGFYLAFQDIGACIALVSVRVFYKKCP |
| | | FTVRNLAMFPDTIPRVDSSSLVEVRGSCVKSAEERDTPKLYCGADGDWLVPLGRCICST |
| | | GYEEIEGSCHACRPGFYKAFAGNTKCSKCPPHSLTYMEATSVCQCEKGYFRAEKDPPS |
| | | MACTRPPSAPRNVVFNINETALILEWSPPSDTGGRKDLTYSVICKKCGLDTSQCEDCGG |
| | | GLRFIPRHTGLINNSVIVLDFVSHVNYTFEIEAMNGVSELSFSPKPFTAITVTTDQDAPSLI |
| | | GVVRKDWASQNSIALSWQAPAFSNGAILDYEIKYYEKEHEQLTYSSTRSKAPSVIITGLK |
| | | PATKYVFHIRVRTATGYSGYSQKFEFETGDETSDMAAEQGQILVIATAAVGGFTLLVILT |
| | | LFFLITGRCQWYIKAKMKSEEKRRNHLQNGHL |
| 56 | EPHA7 | AKEVLLLDSKAQQTELEWISSPPNGWEEISGLDENYTPIRTYQVCQVMEPNQNNWLR |
| | (30-607) | TNWISKGNAQRIFVELKFTLRDCNSLPGVLGTCKETFNLYYYETDYDTGRNIRENLYVKI |
| | | DTIAADESFTQGDLGERKMKLNTEVREIGPLSKKGFYLAFQDVGACIALVSVKVYYKKC |
| | | WSIIENLAIFPDTVTGSEFSSLVEVRGTCVSSAEEEAENAPRMHCSAEGEWLVPIGKCIC |
| | | KAGYQQKGDTCEPCGRGFYKSSSQDLQCSRCPTHSFSDKEGSSRCECEDGYYRAPSDP |
| | | PYVACTRPPSAPQNLIFNINQTTVSLEWSPPADNGGRNDVTYRILCKRCSWEQGECVP |
| | | CGSNIGYMPQQTGLEDNYVTVMDLLAHANYTFEVEAVNGVSDLSRSQRLFAAVSITT |
| | | GQAAPSQVSGVMKERVLQRSVELSWQEPEHPNGVITEYEIKYYEKDQRERTYSTVKTK |
| | | STSASINNLKPGTVYVFQIRAFTAAGYGNYSPRLDVATLEEATGKMFEATAVSSEQNPV |
| | | IIIAVVAVAGTIILVFMVFGFIIGRRHCGYSKADQEGDEELYFHFKFPGTKT |
| 57 | EPHA8 | EVNLLDTSTIHGDWGWLTYPAHGWDSINEVDESFQPIHTYQVCNVMSPNQNNWLR |
| | (31-589) | TSWVPRDGARRVYAEIKFTLRDCNSMPGVLGTCKETFNLYYLESDRDLGASTQESQFLK |
| | | IDTIAADESFTGADLGVRRLKLNTEVRSVGPLSKRGFYLAFQDIGACLAILSLRIYYKKCPA |
| | | MVRNLAAFSEAVTGADSSSLVEVRGQCVRHSEERDTPKMYCSAEGEWLVPIGKCVCS |
| | | AGYEERRDACVACELGFYKSAPGDQLCARCPPHSHSAAPAAQACHCDLSYYRAALDPP |
| | | SSACTRPPSAPVNLISSVNGTSVTLEWAPPLDPGGRSDITYNAVCRRCPWALSRCEACG |
| | | SGTRFVPQQTSLVQASLLVANLLAHMNYSFWIEAVNGVSDLSPEPRRAAVVNITTNQA |
| | | APSQVVVIRQERAGQTSVSLLWQEPEQPNGIILEYEIKYYEKDKEMQSYSTLKAVTTRA |
| | | TVSGLKPGTRYVFQVRARTSAGCGRFSQAMEVETGKPRPRYDTRTIVWICLTLITGLVV |
| | | LLLLLICKKRHCGYSKAFQDSDEEKMHYQNGQA |
| 58 | EPHA10 | EVILLDSKASQAELGWTALPSNGWEEISGVDEHDRPIRTYQVCNVLEPNQDNWLQTG |
| | (35-604) | WISRGRGQRIFVELQFTLRDCSSIPGAAGTCKETFNVYYLETEADLGRGRPRLGGSRPR |
| | | KIDTIAADESFTQGDLGERKMKLNTEVREIGPLSRRGFHLAFQDVGACVALVSVRVYYK |
| | | QCRATVRGLATFPATAAESAFSTLVEVAGTCVAHSEGEPGSPPRMHCGADGEWLVPV |
| | | GRCSCSAGFQERGDFCEACPPGFYKVSPRRPLCSPCPEHSRALENASTFCVCQDSYARS |
| | | PTDPPSASCTRPPSAPRDLQYSLSRSPLVLRLRWLPPADSGGRSDVTYSLLCLRCGREGP |
| | | AGACEPCGPRVAFLPRQAGLRERAATLLHLRPGARYTVRVAALNGVSGPAAAAGTTY |
| | | AQVTVSTGPGAPWEEDEIRRDRVEPQSVSLSWREPIPAGAPGANDTEYEIRYYEKGQS |
| | | EQTYSMVKTGAPTVTVTNLKPATRYVFQIRAASPGPSWEAQSFNPSIEVQTLGEAASG |
| | | SRDQSPAIVVTVVTISALLVLGSVMSVLAIWRRPCSYGKGGGDAHD |
| 59 | EPHB1 | ETLMDTRTATAELGWTANPASGWEEVSGYDENLNTIRTYQVCNVFEPNQNNWLLTT |
| | (21-591) | FINRRGAHRIYTEMRFTVRDCSSLPNVPGSCKETFNLYYYETDSVIATKKSAFWSEAPYL |
| | | KVDTIAADESFSQVDFGGRLMKVNTEVRSFGPLTRNGFYLAFQDYGACMSLLSVRVFF |
| | | KKCPSIVQNFAVFPETMTGAESTSLVIARGTCIPNAEEVDVPIKLYCNGDGEWMVPIGR |
| | | CTCKPGYEPENSVACKACPAGTFKASQEAEGCSHCPSNSRSPAEASPICTCRTGYYRAD |
| | | FDPPEVACTSVPSGPRNVISIVNETSIILEWHPPRETGGRDDVTYNIICKKCRADRRSCSR |
| | | CDDNVEFVPRQLGLTECRVSISSLWAHTPYTFDIQAINGVSSKSPFPPQHVSVNITTNQ |
| | | AAPSTVPIMHQVSATMRSITLSWPQPEQPNGIILDYEIRYYEKEHNEFNSSMARSQTN |
| | | TARIDGLRPGMVYVVQVRARTVAGYGKFSGKMCFQTLTDDDYKSELREQLPLIAGSAA |
| | | AGVVFVVSLVAISIVCSRKRAYSKEAVYSDKLQHYSTGRGSPGM |
| 60 | EPHB2 | VEETLMDSTTATAELGWMVHPPSGWEEVSGYDENMNTIRTYQVCNVFESSQNNWL |
| | (19-589) | RTKFIRRRGAHRIHVEMKFSVRDCSSIPSVPGSCKETFNLYYYEADFDSATKTFPNWME |
| | | NPWVKVDTIAADESFSQVDLGGRVMKINTEVRSFGPVSRSGFYLAFQDYGGCMSLIA |
| | | VRVFYRKCPRIIQNGAIFQETLSGAESTSLVAARGSCIANAEEVDVPIKLYCNGDGEWLV |
| | | PIGRCMCKAGFEAVENGTVCRGCPSGTFKANQGDEACTHCPINSRTTSEGATNCVCR |
| | | NGYYRADLDPLDMPCTTIPSAPQAVISSVNETSLMLEWTPPRDSGGREDLVYNIICKSC |
| | | GSGRGACTRCGDNVQYAPROLGLTEPRIYISDLLAHTQYTFEIQAVNGVTDQSPFSPQF |
| | | ASVNITTNQAAPSAVSIMHQVSRTVDSITLSWSQPDQPNGVILDYELQYYEKELSEYNA |
| | | TAIKSPTNTVTVQGLKAGAIYVFQVRARTVAGYGRYSGKMYFQTMTEAEYQTSIQEKL |
| | | PLIIGSSAAGLVFLIAVVVIAIVCNRRGFERADSEYTDKLQHYTSGHM |
| 61 | EPHB3 | EETLMDTKWVTSELAWTSHPESGWEEVSGYDEAMNPIRTYQVCNVRESSQNNWLR |
| | (39-605) | TGFIWRRDVQRVYVELKFTVRDCNSIPNIPGSCKETFNLFYYEADSDVASASSPFWME |
| | | NPYVKVDTIAPDESFSRLDAGRVNTKVRSFGPLSKAGFYLAFQDQGACMSLISVRAFYK |
| | | KCASTTAGFALFPETLTGAEPTSLVIAPGTCIPNAVEVSVPLKLYCNGDGEWMVPVGAC |
| | | TCATGHEPAAKESQCRPCPPGSYKAKQGEGPCLPCPPNSRTTSPAASICTCHNNFYRA |
| | | DSDSADSACTTVPSPPRGVISNVNETSLILEWSEPRDLGGRDDLLYNVICKKCHGAGGA |
| | | SACSRCDDNVEFVPRQLGLTERRVHISHLLAHTRYTFEVQAVNGVSGKSPLPPRYAAV |
| | | NITTNQAAPSEVPTLRLHSSSGSSLTLSWAPPERPNGVILDYEMKYFEKSEGIASTVTSQ |
| | | MNSVQLDGLRPDARYVVQVRARTVAGYGQYSRPAEFETTSERGSGAQQLQEQLPLIV |
| | | GSATAGLVFVVAVVVIAIVCLRKQRHGSDSEYTEKLQQYIAPGM |
| 62 | EPHB4 | EETLLNTKLETADLKWVTFPQVDGQWEELSGLDEEQHSVRTYEVCDVQRAPGQAHW |
| | (19-583) | LRTGWVPRRGAVHVYATLRFTMLECLSLPRAGRSCKETFTVFYYESDADTATALTPAW |
| | | MENPYIKVDTVAAEHLTRKRPGAEATGKVNVKTLRLGPLSKAGFYLAFQDQGACMAL |
| | | LSLHLFYKKCAQLTVNLTRFPETVPRELVVPVAGSCVVDAVPAPGPSPSLYCREDGQW |
| | | AEQPVTGCSCAPGFEAAEGNTKCRACAQGTFKPLSGEGSCQPCPANSHSNTIGSAVC |
| | | QCRVGYFRARTDPRGAPCTTPPSAPRSVVSRLNGSSLHLEWSAPLESGGREDLTYALRC |
| | | RECRPGGSCAPCGGDLTFDPGPRDLVEPWVVVRGLRPDFTYTFEVTALNGVSSLATGP |
| | | VPFEPVNVTTDREVPPAVSDIRVTRSSPSSLSLAWAVPRAPSGAVLDYEVKYHEKGAEG |
| | | PSSVRFLKTSENRAELRGLKRGASYLVQVRARSEAGYGPFGQEHHSQTQLDESEGWRE |
| | | QLALIAGTAVVGVVLVLVVIVVAVLCLRKQSNGREAEYSDKHGQYLI |
| 63 | EPHB6 | EEVLLDTTGETSEIGWLTYPPGGWDEVSVLDDQRRLTRTFEACHVAGAPPGTGQDN |
| | (33-649) | WLQTHFVERRGAQRAHIRLHFSVRACSSLGVSGGTCRETFTLYYRQAEEPDSPDSVSS |
| | | WHLKRWTKVDTIAADESFPSSSSSSSSSSSAAWAVGPHGAGQRAGLQLNVKERSFGP |
| | | LTQRGFYVAFQDTGACLALVAVRLFSYTCPAVLRSFASFPETQASGAGGASLVAAVGTC |
| | | VAHAEPEEDGVGGQAGGSPPRLHCNGEGKWMVAVGGCRCQPGYQPARGDKACQ |
| | | ACPRGLYKSSAGNAPCSPCPARSHAPNPAAPVCPCLEGFYRASSDPPEAPCTGPPSAP |
| | | QELWFEVQGSALMLHWRLPRELGGRGDLLFNVVCKECEGRQEPASGGGGTCHRCRD |
| | | EVHFDPRQRGLTESRVLVGGLRAHVPYILEVQAVNGVSELSPDPPQAAAINVSTSHEVP |
| | | SAVPVVHQVSRASNSITVSWPQPDQTNGNILDYQLRYYDQAEDESHSFTLTSETNTAT |
| | | VTQLSPGHIYGFQVRARTAAGHGPYGGKVYFQTLPQGELSSQLPERLSLVIGSILGALAF |
| | | LLLAAITVLAVVFQRKRRGTGYTEQLQQYSSPGLGVKYYIDPS |

When a transmembrane region of a Type II transmembrane protein is employed as a scaffold protein (referred to as T2 Scaffold), a FcRn-binding site preferably comprises an arrangement of domains wherein the Type II-derived transmembrane domain is N-terminal to the FcRn-binding site. For example, a FcRn fusion protein may comprise an arrangement of domains wherein, in an N-terminal to C-terminal direction, a scaffold protein or fragment thereof comprising a Type II-derived transmembrane domain is linked to a CH2 domain (of the modified Fc domain), which in turn is linked to a monomeric CH3 domain (of the modified Fc domain) (as depicted in FIG. 2).

Thus, the most common configurations of a FcRn binding polypeptide of the present disclosure that comprise a Type II-derived transmembrane domain will contain an N-terminal to C-terminal linear structure illustrated as follows:

(T2 Scaffold)-L-(FcRn binding site), (2)

where L in the formulae represents a direct peptide bond linking two domains or a linker sequence of one or more amino acid residues.

As a non-limiting example, FcRn binding polypeptides are constructed by fusing a modified Fc polypeptide to the C-terminal of polytopic Type II derived transmembrane protein scaffold (T2 Scaffold) of AT1B3 (Uniprot P54709) which shares at least about 70%, at least about 80%, at least about 85%, at least about 90%, at least about 95%, at least about 96%, at least about 97%, at least about 98%, or at least about 99% sequence identity with AT1B3 according to SEQ ID NO: 64 or with a functional fragment thereof

| | | |
|---|---|---|
| 64 | AT1B3 - | MTKNEKKSLNQSLAEWKLFIYNPTTGEFLGRTAKSWGL |
| | L - | ILLFYLVFYGFLAALFSFTMWVMLQTLNDEVPKYRDQI |
| | monoFC | PSPGLMVFPKPVTALEYTFSRSDPTSYAGYIEDLKKFL |
| | | KPYTLEEQKNLTVCPDGALFEQKGPVYVACQFPISLLQ |
| | | ACSGMNDPDFGYSQGNPCILVKMNRIIGLKPEGVPRID |
| | | CVSKNEDIPNVAVYPHNGMIDLKYFPYYGKKLHVGYLQ |
| | | PLVAVQVSFAPNNTGKEVTVECKIDGSANLKSQDDRDK |
| | | FLGRVMFKITARAGGGSGGGGSGGGGSGGGGSGGAPEA |
| | | AGGPSVFLFPPKPKDTLMISRTPEVTCVVVDVSHEDPE |
| | | VKFNWYVDGVEVHNAKTKPREEQYNSTYRVVSVLTVLH |
| | | QDWLNGKEYKCKVSNKALPAPIEKTISKAKGQPREPQV |
| | | YTKSPSRDELTKNQVSLSCHVKGFYPSDIAVLWESYGT |
| | | EWSSYKTTVPVLDSDGSFRLASYLTVTKEEWQQGFVFS |
| | | CSVMHEALHNHYTQKSLSLSPGK |

In certain embodiments, when a transmembrane region from a multi-span (polytopic) transmembrane protein is employed as a scaffold protein (referred to as PT scaffold), the location of the transmembrane domain with respect to the FcRn binding site will vary according to how many membrane-spanning regions of the transmembrane region are selected and what is the orientation of the membrane-spanning region(s) selected, N-terminal to C-terminal, relative to the cytoplasmic and ER sides of the cellular membrane (as depicted in FIG. 2). Exemplary PT scaffolds with non-cytoplasmic termini are listed in Table 3. It will be clear to the skilled person which terminus of the PT scaffold or fragment thereof is non-cytoplasmic.

Accordingly, possible linear configurations for a FcRn binding polypeptide of the disclosure utilizing transmembrane domains derived from scaffold proteins (PT scaffold) comprising polytopic transmembrane domain may be illustrated as follows and may include the use of plural transmembrane domains:

(FcRn binding site)-L-(PT Scaffold), and/or (3)

(PT Scaffold)-L-(FcRn binding site), (4)

where L in each formulae represents a direct peptide bond linking two domains or a linker of one or more amino acid residues.

In some embodiments a PT scaffold protein comprising polytopic transmembrane domains is located at the C-terminal domain relative to FcRn binding site, similar to the arrangement for using a scaffold protein comprising a Type I-derived transmembrane domain, while in other embodiments, a PT scaffold protein comprising polytopic transmembrane domains is located at the N-terminal domain relative to FcRn binding site. Unlike scaffold proteins comprising a Type I transmembrane domain, scaffold proteins comprising a PT transmembrane may not require an N-terminal signal sequence to direct the N-terminus of the PT scaffold into the ER membrane and through to the ER lumen. For a FcRn-binding polypeptide protein comprising a PT-derived transmembrane domain, however, an N-terminal signal sequence may still be required to achieve the desired position of the FcRn binding polypeptide on the nanovesicle surface.

As a non-limiting example, FcRn binding polypeptides are constructed by fusing a modified Fc polypeptide to the C-terminal of a polytopic transmembrane domain derived from protein scaffold of Zip2 (including a modification to reduce metal transport at position H63A relative to the wildtype sequence) which shares at least about 70%, at least about 80%, at least about 85%, at least about 90%, at least about 95%, at least about 96%, at least about 97%, at least about 98%, or at least about 99% sequence identity with Zip2 according to SEQ ID NO: 65 or with a functional fragment thereof.

As an additional non-limiting example, FcRn binding polypeptides are constructed by fusing a modified Fc polypeptide to the N-terminal of PT scaffold of Zip2 (including a modification to reduce metal transport at position H63A relative to the wildtype sequence) which shares at least about 70%, at least about 80%, at least about 85%, at least about 90%, at least about 95%, at least about 96%, at least about 97%, at least about 98%, or at least about 99% sequence identity with Zip2 according to SEQ ID NO: 66 or with a functional fragment thereof.

As a further non-limiting example, FcRn binding polypeptides are constructed by fusing a modified Fc polypeptide to both the N-terminal and C-terminal of a PT scaffold of Zip2 (including a modification to reduce metal transport at position H63A relative to the wildtype sequence) as both termini are located at the surface of the nanovesicle which shares at least about 70%, at least about 80%, at least about 85%, at least about 90%, at least about 95%, at least about 96%, at least about 97% at least about 98%, or at least about 99% sequence identity with Zip2 according to SEQ ID NO: 67 or with a functional fragment thereof.

| | | |
|---|---|---|
| 65 | Zip2 + L + C- | MEQLLGIKLGCLFALLALTLGCGLTPICFKWFQIDAARGHHRLVLRLLGCISAGVF |
| | terminal | LGAGFMAMTAEALEEIESQIQKFMVQNRSASERNSSGDADSAHMEYPYGELII |
| | monoFC | SLGFFFVFFLESLALQCCPGAAGGSTVQDEEWGGAHIFELHSHGHLPSPSKGPL |
| | | RALVLLLSLSFHSVFEGLAVGLQPTVAATVQLCLAVLAHKGLVVFGVGMRLVHL |
| | | GTSSRWAVFSILLLALMSPLGLAVGLAVTGGDSEGGRGLAQAVLEGVAAGTFLY |
| | | VTFLEILPRELASPEAPLAKWSCVAAGFAFMAFIALWAGGGSGGGGSGGGGSG |
| | | GGGSGGAPEAAGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSNKALG |
| | | APIEKTISKAKGQPREPQVYTKPPSRDELTKNQVSLSCLVKGFYPSDIAVEWESN |
| | | GQPENNYKTTVPVLDSDGSFRLASYLTVDKSRWQQGNVFSCSVMHEALHNHY |
| | | TQKSLSLSP |
| 66 | N-terminal | MAPEAAGGPSVFLFPPKPKDTLMISRTPEVTCVVVDVSHEDPEVKFNWYVDGV |
| | monoFC + L + | EVHNAKTKPREEQYNSTYRVVSVLTVLHQDWLNGKEYKCKVSNKALGAPIEKTI |
| | Zip2 | SKAKGQPREPQVYTKPPSRDELTKNQVSLSCLVKGFYPSDIAVEWESNGQPEN |
| | | NYKTTVPVLDSDGSFRLASYLTVDKSRWQQGNVFSCSVMHEALHNHYTQKSLS |
| | | LSPGGGSGGGGSGGGGSGGGGSGGEQLLGIKLGCLFALLALTLGCGLTPICFKW |
| | | FQIDAARGHHRLVLRLLGCISAGVFLGAGFMAMTAEALEEIESQIQKFMVQNR |
| | | SASERNSSGDADSAHMEYPYGELIISLGFFFVFFLESLALQCCPGAAGGSTVQDE |
| | | EWGGAHIFELHSHGHLPSPSKGPLRALVLLLSLSFHSVFEGLAVGLQPTVAATVQ |
| | | LCLAVLAHKGLVVFGVGMRLVHLGTSSRWAVFSILLLALMSPLGLAVGLAVTGG |
| | | DSEGGRGLAQAVLEGVAAGTFLYVTFLEILPRELASPEAPLAKWSCVAAGFAFM |
| | | AFIALWA |
| 67 | N-terminal | MAPEAAGGPSVFLFPPKPKDTLMISRTPEVTCVVVDVSHEDPEVKFNWYVDGV |
| | monoFC + L + | EVHNAKTKPREEQYNSTYRVVSVLTVLHQDWLNGKEYKCKVSNKALGAPIEKTI |
| | Zip2 + L + C- | SKAKGQPREPQVYTKPPSRDELTKNQVSLSCLVKGFYPSDIAVEWESNGQPEN |
| | terminal | NYKTTVPVLDSDGSFRLASYLTVDKSRWQQGNVFSCSVMHEALHNHYTQKSLS |
| | monoFc | LSPGGGSGGGGSGGGGSGGGGSGGEQLLGIKLGCLFALLALTLGCGLTPICFKW |
| | | FQIDAARGHHRLVLRLLGCISAGVFLGAGFMAMTAEALEEIESQIQKFMVQNR |
| | | SASERNSSGDADSAHMEYPYGELIISLGFFFVFFLESLALQCCPGAAGGSTVQDE |
| | | EWGGAHIFELHSHGHLPSPSKGPLRALVLLLSLSFHSVFEGLAVGLQPTVAATVQ |
| | | LCLAVLAHKGLVVFGVGMRLVHLGTSSRWAVFSILLLALMSPLGLAVGLAVTGG |
| | | DSEGGRGLAQAVLEGVAAGTFLYVTFLEILPRELASPEAPLAKWSCVAAGFAFM |
| | | AFIALWAGGGSGGGGSGGGGSGGGGSGGAPEAAGGPSVFLFPPKPKDTLMIS |
| | | RTPEVTCVVVDVSHEDPEVKFNWYVDGVEVHNAKTKPREEQYNSTYRVVSVLT |
| | | VLHQDWLNGKEYKCKVSNKALGAPIEKTISKAKGQPREPQVYTKPPSRDELTKN |
| | | QVSLSCLVKGFYPSDIAVEWESNGQPENNYKTTVPVLDSDGSFRLASYLTVDKS |
| | | RWQQGNVFSCSVMHEALHNHYTQKSLSLSP |

In certain embodiments, when a transmembrane region from a multi-span (polytopic) scaffold protein comprising both N- and C-termini that are oriented towards cytoplasm, the FcRn binding site can be placed on an extracellular loop in between two adjacent membrane-spanning fragments of the transmembrane domain. Exemplary PT scaffolds with cytoplasmic termini are listed in Table 4. It will be clear to the skilled person which loops from the PT scaffold transmembrane domain are extracellular as opposed to cytoplasmic.

Accordingly, possible linear configurations for a FcRn binding polypeptide of the disclosure utilizing transmembrane domains derived from scaffold proteins (PT scaffolds) comprising a polytopic transmembrane domain (as depicted in FIG. 2) may be illustrated as follows and may include the use of plural transmembrane domains:

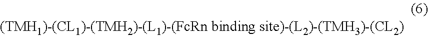

where each L in the formulae represents a direct peptide bond linking two domains or a linker of one or more amino acid residues and TMH denotes a “transmembrane helix” and CL denotes a “cytoplasmic loop”.

### (c) Standard Assays to Determine Polypeptides Useful as Scaffold Proteins

In addition to the particular features of the disclosure elucidated in the examples below, it is evident that proteins comprising a transmembrane region, can be employed in assays to determine whether or not a particular scaffold protein is useful as a FcRn binding polypeptide according to the disclosure. In such a scaffold protein assay, a recombinant nucleic acid molecule is produced by standard methods (for example, nucleic acid synthesis, recombinant DNA techniques, and/or polymerase change reaction (PCR) methods) that encodes the amino acid sequence of a fusion protein comprising a FcRn binding site fused in frame with a candidate transmembrane domain. The candidate scaffold protein comprises a portion of a membrane protein that normally resides in or traverses a cellular or intracellular membrane in accordance with the features of a transmembrane domain described herein. Thus, by way of non-limiting example, in order to test or assess any candidate polypeptide as a scaffold protein, a nucleic acid encoding the candidate transmembrane domain is linked in frame to a nucleic acid encoding the common portion of a FcRn binding polypeptide comprising a FcRn binding site. The resulting recombinant nucleic acid encoding the candidate FcRn binding polypeptide fusion protein can then be inserted into an expression vector. Cells of a mammalian cell line, such as HEK 293 cells used in the examples below, can be transfected with the expression vectors. The transfected cells can then be isolated and grown in culture under conditions that permit expression of the proteins encoded on the expression vectors. Samples of the culture media or nanovesicles isolated therefrom, can be assayed for the amount of nanovesicle (e.g. EV) anchored FcRn binding polypeptides, (for example using enzyme linked immunosorbent assay (ELISA)), flow cytometry (e.g. using a fluorescent anti-Fc domain antibody) or functional binding to FcRn in acidic pH (e.g. using the Lumit™ FcRn competition assay). An enhancement in the level of FcRn binding polypeptide in the media of transfected cells as compared to the level of FcRn binding polypeptide in the media of untransfected control cells indicates that the scaffold protein, and therefore the candidate FcRn binding polypeptide is useful as scaffold in accordance with the disclosure. Preferably, the level of FcRn binding polypeptide present in nanovesicles secreted into the media of cultures of cells expressing the fusion protein is at least 1.5-fold higher than that of the level in the media of control cells. Enhancing the level of FcRn binding polypeptide secreted from nanovesicles (e.g. exosomes) is also a therapeutically and commercially important property and an increase by 1.5-fold or more can provide a significant reduction in production costs and a significant increase in the availability of the therapeutically and commercially important nanovesicles.

In some embodiments, the FcRn binding polypeptides comprising the FcRn binding site and a scaffold protein may also contain additional polypeptide domains or sequences. Such additional polypeptide domains may exert various functions, for instance such domains may (i) contribute to increasing the surface concentration of the FcRn binding polypeptide (ii) lead to clustering of the scaffold proteins thereby increasing the avidity of the FcRn binding polypeptides, (iii) function as linkers to optimize the interaction between the scaffold proteins and the FcRn binding site, and/or (iv) improve anchoring in the nanovesicle membrane, as well as various other functions.

### 5.2.4 Functional Moieties

In a further aspect, FcRn binding polypeptide provided herein, in addition to being able to conditionally bind FcRn, can also comprise one or more functional moieties (e.g., fusion moieties, preferably a targeting domain that is capable of targeting a nanovesicle (e.g., EV or hybridosome) comprising the polypeptide to a specific organ, tissue, or cell type. In a preferred embodiment, the one or more functional moieties are proteins (e.g., peptides or polypeptides). In a preferred embodiment, the one or more functional moieties are fused in-frame to the remaining portion of the polypeptide. In certain embodiments, the one or more functional moieties are covalently fused to the remaining portion of the polypeptide via a linker.

Such one or more functional moieties can be N- or C-terminal to (e.g., N-terminally and/or C-terminally fused to) the remaining portion of the polypeptide or placed between the different domains of the remaining portion of the polypeptide. In certain embodiments, the one or more functional moieties are presented towards the external space of a nanovesicle. In some embodiments, the one or more functional moieties are N-terminal to (e.g., N-terminally fused to) the transmembrane domain of the scaffold protein. In some embodiments, the one or more functional moieties are N-terminal to (e.g., N-terminally fused to) the modified Fc domain. In some embodiments, the one or more functional moieties are C-terminal to (e.g., N-terminally fused to) the modified Fc domain. In some embodiments, the one or more functional moieties are C-terminal to (e.g., C-terminally fused to) the transmembrane domain of the scaffold protein. In some embodiments, the one or more functional moieties are N-terminal to (e.g., N-terminally fused to) the transmembrane domain of the scaffold protein. In certain embodiments, the one or more functional moieties are presented towards the lumen of a nanovesicle. In some embodiments, the one or more functional moieties are C-terminal to (e.g., C-terminally fused to) the transmembrane domain of the scaffold protein. In some embodiments, the one or more functional moieties are C-terminal to (e.g., C-terminally fused to) the modified Fc domain.

Exemplary functional moieties include, without being limited to, targeting domains and purification domains such as affinity tags. The functional moieties may be a large polypeptide or a peptide. In some embodiments, a FcRn binding polypeptide comprises a FcRn binding site and optionally a targeting moiety, each of which can be independently modified.

In some embodiments targeting domains are preferably located on the surface of a nanovesicle. A targeting domain aids directing the nanovesicle towards a specific organ, tissue, or cell and is preferably specific to an organ, a tissue, or a cell. One or more targeting domains may be fused to the remaining portion of the FcRn binding polypeptide. The presence of more than one targeting domain may increase specificity for the targeted organ, tissue, or cell. In some embodiments, the targeting domain is or comprises one or more antigen binding molecules. In some embodiments, the targeting domain specifically targets an antigen expressed on cancer, metastatic, dendritic, stem or immunological cell. Exemplary antigens expressed on tumor cells include, without being limited to, BAGE, BCMA, CEA, CD19, CD20, CD33, CD123, CEA, FAP, HER2, LMP1, LMP2, MAGE, Mart1/MelanA, NY-ESO, PSA, PSMA, RAGE and survivin.

In some embodiments targeting domains are located in the lumen of a nanovesicle. A targeting domain aids attaching cytoplasmic components (e.g. proteins, protein-complex, viruses) to the scaffold prior to invagination and vesicle formation. The presence of more than one targeting domain may increase loading efficiency of cytoplasmic components into the lumen of the nanovesicle during biogenesis. In some embodiments, the targeting domain is or comprises one or more antigen binding molecules. In some embodiments, the targeting domain specifically targets an antigen expressed on adeno-associated viruses.

In certain embodiments, the targeting domain is selected from the group consisting of: scFv, (scFv)2, Fab, Fab′, F(ab′)2, Fv, dAb, Fd fragments, diabodies, F(ab′)3, disulfide linked Fv, sdAb (VHH or nanobody), CDR, di-scFv, bi-scFv, tascFv (tandem scFv), triabody, tetrabody, V-NAR domain, Fcab, IgGACH2, DVD-Ig, probody, a DARPin, a Centyrin, an affibody, an affilin, an affitin, an anticalin, an avimer, a Fynomer, a Kunitz domain peptide, a monobody (or adnectin), a tribody, and a nanofitin. In certain embodiments, the targeting domain is selected from the group consisting of: scFv, (scFv)₂, Fab, Fab′, F(ab′)2, F(abl)₂, Fv, dAb, Fd fragments, diabodies, F(ab)2, F(ab′), F(ab′)3, Fd, Fv, disulfide linked Fv, dAb, sdAb, nanobody, CDR, di-scFv, bi-scFv, tascFv (tandem scFv), AVIBODY (e.g., diabody, triabody, tetrabody), T-cell engager (BiTE), V-NAR domain, Fcab, IgGACH2, DVD-Ig, probody, intrabody, DARPin, Centyrin, affibody, affilin, affitin, anticalin, avimer, Fynomer, Kunitz domain peptide, monobody, adnectin, tribody, and nanofitin.

In certain embodiments, the targeting domain specifically binds to a marker. In specific embodiments, the marker is a tumor-associated antigen. In a specific embodiment, the tumor-associated antigen is selected from the group consisting of human epidermal growth factor receptor 2 (HER2), CD20, CD33, B-cell maturation antigen (BCMA), prostate-specific membrane (PSMA), DLL3, ganglioside GD2 (GD2), CD 123, anoctamin-1 (Ano1), mesothelin, carbonic anhydrase IX (CAIX), tumor-associated calcium signal transducer 2 (TROP2), carcinoembryonic antigen (CEA), claudin-18.2, receptor tyrosine kinase-like orphan receptor 1 (ROR1), trophoblast glycoprotein (5T4), glycoprotein nonmetastatic melanoma protein B (GPNMB), folate receptor-alpha (FR-alpha), pregnancy-associated plasma protein A (PAPP-A), CD37, epithelial cell adhesion molecule (EpCAM), CD2, CD 19, CD30, CD38, CD40, CD52, CD70, CD79b, fms-like tyrosine kinase 3 (FLT3), glypican 3 (GPC3), B7 homolog 6 (B7H6), C—C chemokine receptor type 4 (CCR4), C—X—C motif chemokine receptor 4 (CXCR4), receptor tyrosine kinase-like orphan receptor 2 (ROR2), CD133, HLA class I histocompatibility antigen, alpha chain E (HLA-E), epidermal growth factor receptor (EGFR/ERBB-1), insulin like growth factor 1-receptor (IGF1R), and human epidermal growth factor receptor 3.

In some aspects, methods of targeting nanovesicles to a specific organ, tissue or cell are provided, comprising the steps of fusing a targeting domain to the portion of a FcRn binding polypeptide of the disclosure and getting the polypeptide expressed in nanovesicles.

Antigen binding molecules serving as targeting domains, may be monospecific, bispecific or multispecific, i.e., they may target one or more epitopes of the same target or different targets. The more specificities that are displayed on the nanovesicle, the more specific its targeting is. In some embodiments, the antigen binding molecule is selected from the group consisting of:
i) a full-length antibody molecule (such as an IgG, an IgM, an IgA, an IgM or an IgE);
ii) an antibody fragment such as a CDR, a Dab, a Fab, a Fab′, a F(ab)′2, a Fd fragment, a Fv fragment, a disulfide linked Fv, a scFab, a nanobody, a minimal recognition unit, a VHH or a V-NAR domain;
iii) a non-antibody scaffold such as an affibody, an affitin molecule, an affitin, an AdNectin, an anticalin, an avimer, a centyrin, a lipocalin mutein, a DARPin, a fynomer, a Knottin, a Kunitz-type domain, a nanofitin, a tetranectin or a trans-body;
iv) a fusion polypeptide comprising one or more antibody domains, such as a bi-scFv, aBITE, a diabody, di-scFv, probody, tascFv (tandem scFv), triabody, tribody, tetrabody, IgGACH2, DVD-Ig, MATCH, a minibody, a scFv, a scFv-Fc, bispecific F(ab′)2, F(ab′)3, monovalent IgG;
v) a soluble T-cell receptor (sTCR);
vi) a peptide, such as natural peptide, a recombinant peptide, a synthetic peptide; and
vii) a viral protein such as the receptor binding domain of a viral spike protein (such as of coronavirus) or hemagglutinin (HA) of influenza, Nipah virus protein F, a measles virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein, or fragments thereof, respectively.

In a further aspect, knowing that a transmembrane region is derived from a particular type of transmembrane protein suggests a preferred orientation and location for the transmembrane domain relative to the targeting moiety in the FcRn-binding polypeptide of the disclosure.

Thus, the most common configurations of a FcRn binding polypeptide of the present disclosure that have a Type I-derived transmembrane domain will comprise an N-terminal to C-terminal linear structure illustrated as follows:

(targeting domain)-L-(FcRn binding site)-L-(T1 Scaffold),

where each L in the formulae represents a direct peptide bond linking two domains or a linker of one or more amino acid residues.

In contrast, in embodiments in which a transmembrane region of a Type II transmembrane protein is employed as a transmembrane domain, the arrangement of domains results in configurations of a targeting moiety fused to a FcRn binding polypeptide of the present disclosure may comprise an N-terminal to C-terminal linear structure illustrated as follows:

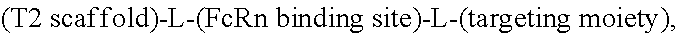

where each L in the formulae represents a direct peptide bond linking two domains or a linker of one or more amino acid residues.

In some embodiments, the FcRn binding polypeptide comprises targeting moiety that is a bispecific modified Fc domain (e.g., a Fc domain further modified to promote transferrin receptor binding). In some embodiments, bispecific modified Fe domains comprise monomeric CH3 domains modified to display higher binding affinity to the transferrin receptor compared to non-modified CH3 domains and retains the ability to bind the Fc binding site of FcRn. In some embodiments, a modified Fc domain that specifically binds to the transferrin receptor comprises one, two, three, four, five, six, seven, eight, nine, ten, or eleven substitutions in a set of amino acid positions comprising 380, 384, 386, 387, 388, 389, 390, 413, 415, 416, and 421, according to EU numbering. In some embodiment, a modified Fc domain that specifically binds to the transferrin receptor is at least 80%, 90% or 95% similar to SEQ ID 34.

### 5.3 Nucleic Acids, Expression Vectors, Cells, and Methods of Making a Polypeptide

Also provided herein are nucleic acids encoding a polypeptide described herein (e.g., described in Section 5.2), vectors (e.g., expression vectors) comprising a nucleic acid described herein, and cells (e.g., host cells) comprising a nucleic acid or expression vector described herein.

The FcRn binding polypeptides of the disclosure can be produced using any number of expression systems, including prokaryotic and eukaryotic expression systems. In some embodiments, the expression system is a mammalian cell expression system, such as HEK293T systems. Many such systems are widely available from commercial suppliers. In some embodiments, the polynucleotides encoding the polypeptides (in particular, the FcRn binding polypeptides) may be expressed using a single vector, e.g, in a bi-cistronic expression unit, or under the control of different promoters. In other embodiments, the polynucleotides encoding the polypeptides (in particular, the FcRn binding polypeptides)) may be expressed using separate vectors.

The polynucleotides may be present in various different forms and/or in different vectors. For instance, the polynucleotides may be essentially linear, circular, and/or have any secondary and/or tertiary and/or higher order structure. Furthermore, the present disclosure also relates to vectors comprising the polynucleotides, e.g. vectors such as plasmids, any circular or linear DNA polynucleotide, mini-circles, viruses (such as adenoviruses, adeno-associated viruses, lentiviruses, retroviruses), mRNAs, and/or modified mRNAs.

In some aspects, the disclosure provides isolated nucleic acids comprising a nucleic acid sequence encoding any of the polypeptides (in particular, the FcRn binding polypeptides) as described herein; vectors comprising such nucleic acids; and host cells into which the nucleic acids are introduced that are used to replicate the nucleic acids and/or to express the polypeptides (in particular, the FcRn binding polypeptides).

In some embodiments, a polynucleotide (e.g., an isolated polynucleotide) comprises a nucleotide sequence encoding a polypeptide (in particular, the FcRn binding polypeptides) as disclosed herein (e.g., as described above). In some embodiments, a polynucleotide as described herein is operably linked to a heterologous nucleic acid, e.g., a heterologous promoter.

Suitable vectors containing polynucleotides encoding polypeptides (in particular, the FcRn binding polypeptides) of the present disclosure, or fragments thereof, include cloning vectors and expression vectors. While the cloning vector selected may vary according to the cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicate in the cells either as an episome or as an integral part of the chromosomal DNA. Suitable expression vectors include but are not limited to plasmids, viral vectors, including adenoviruses, adeno-associated viruses, lentiviruses, retroviruses, and any other vector. Typically, the coding sequence of the polypeptide is operably linked to a suitable control sequence capable of affecting expression of the DNA in a suitable host. Such a control sequences may include a promoter to affect transcription, an optional operator sequence to control transcription, a sequence encoding suitable ribosome binding sites on the mRNA, enhancers and/or sequences which control termination of transcription and translation.

Suitable cells for cloning or expressing a polynucleotide or vector as described herein include prokaryotic or eukaryotic cells. In some embodiments, the cell is prokaryotic. In some embodiments, the cell is eukaryotic, e.g., a Chinese Hamster Ovary (CHO) cell or lymphoid cell. In some embodiments, the cell is a human cell, e.g., a Human Embryonic Kidney (HEK) cell. In some embodiments, the cell is a human cell, e.g., a Human Embryonic Kidney (HEK) cell. In some embodiments, the cell is non-tumor cell line derived from human amniocytes

Transfection is the process of introducing nucleic acids into cells by non-viral methods. Transduction is the process whereby foreign DNA is introduced into another cell via a viral vector. Common transfection methods include calcium phosphate, cationic polymers (such as PEI), magnetic beads, electroporation and commercial lipid-based reagents such as Lipofectamine and Fugene. Transduction is mostly used to describe the introduction of recombinant viral vector particles into target cells, while ‘infection’ refers to natural infections of humans or animals with wild-type viruses.

Further to the above-mentioned standard methods of nucleic acid delivery, the nucleic acids provided herein can be targeted to specific sites within the genome of the cell. Such methods include, but are not limited to, CRISPR-Cas9, TALENs, meganucleases designed against a genomic sequence of interest within the host cell, and other technologies for precise editing of genomes, Cre-lox site-specific recombination; zinc-finger mediated integration; and homologous recombination. The nucleic acid may contain a transposon comprising a nucleic acid encoding the polypeptides of the disclosure. In some embodiments, said nucleic acid may further contain a nucleic acid sequence encoding a transposase enzyme. In other embodiments, a system with two nucleic acids is provided wherein a first plasmid contains a transposon comprising a nucleic acid encoding the polypeptides of the disclosure, and a second plasmid contains a nucleic acid sequence encoding a transposase enzyme. Both the first and the second nucleic acids may be co-delivered into a host cell. Cells expressing a polypeptide (in particular, an FcRn binding polypeptide) described herein may also be generated by using a combination of gene insertion (using a transposon) and genetic editing (using a nuclease). Exemplary transposons include, but are not limited to, piggyBac and the Sleeping Beauty transposon system (SBTS); whereas exemplary nucleases include, without being limited to, the CRISPR/Cas system, Transcription Activator-Like Effector Nucleases (TALENs) and Zinc finger nucleases (ZFNs).

The genetically-modified cell can contain the exogenous sequences by transient or stable transformation. The exogenous sequences can be transformed as a plasmid. The exogenous sequences can be stably integrated into a genomic sequence of the cell, at a targeted site or in a random site. In some aspects, a stable cell line is generated for production of nanovesicles (e.g., EVs and hybridosomes) comprising polypeptides (in particular, the FcRn binding polypeptides) disclosed herein. Preferably, the cells are stably transfected with the construct encoding the polypeptide (in particular, the FcRn binding polypeptide) of the disclosure, such that a stable cell line is generated. This advantageously results in consistent production of nanovesicles (e.g., EVs and hybridosomes) of uniform quality and yield.

The exogenous sequences encoding for a fragment of polypeptide described herein (in particular, an fragment comprising a FcRn binding site) can be inserted into a genomic sequence of the producer cell, located within, upstream (5′-end) or downstream (3′-end) of an endogenous sequence. Various methods known in the art can be used for the introduction of the exogenous sequences into the producer cell. For example, cells modified using various gene editing methods (e.g., methods using a homologous recombination, transposon-mediated system, loxP-Cre system, CRISPR/Cas9 or TALEN) are within the scope of the present disclosure.

The exogenous nucleic acid sequences can comprise a sequence encoding a polypeptide (in particular, an FcRn binding polypeptide) disclosed herein or a fragment or variant thereof. An extra copy of the sequence encoding a polypeptide (in particular, an FcRn binding polypeptide) can be introduced to produce a nanovesicle described herein (e.g., a nanovesicle having a higher density of a FcRn binding polypeptide or expressing multiple different FcRn binding polypeptide on the surface of the nanovesicle). Exogenous sequences encoding a polypeptide (in particular, an FcRn binding polypeptide), a variant or a fragment thereof, can be introduced to produce a lumen-engineered and/or surface-decorated nanovesicle (EV or hybridosome) and optionally a nanovesicle containing the modification or the fragment of the polypeptide (in particular, the FcRn binding polypeptide).

In some aspects, a cell can be modified, e.g., transfected, with one or more vectors encoding one or more polypeptides (in particular, one or more FcRn binding polypeptides comprising different scaffold proteins) comprising exogenous fusion moieties described herein (e.g., targeting moiety or purification domain).

In another aspect, methods of making a polypeptide (in particular, an FcRn binding polypeptide) as described herein are provided. In some embodiments, the method comprises culturing a host cell as described herein (e.g., a cell comprising a nucleic acid or expression vector as described herein) under conditions suitable for expression of the polypeptide (in particular, the FcRn binding polypeptide). In some embodiments, the polypeptide (in particular, the FcRn binding polypeptide) is subsequently recovered from the host cell (or host cell culture medium). In some embodiments, the polypeptide (in particular, the FcRn binding polypeptide) is purified, e.g., by affinity chromatography.

### 5.4 Nanovesicles (e.g., Extracellular Vesicles and Hybridosomes) and Methods of Producing Nanovesicles

Also provided herein are nanovesicles (e.g., extracellular vesicles and hybridosomes) comprising a polypeptide described herein (e.g., described in Section 5.2). Another aspect of the present disclosure relates to generation and use of surface-engineered nanovesicles. Nanovesicles comprising the polypeptides (in particular, the FcRn binding polypeptides) described herein provide important advancements and lead to novel nanovesicle compositions and methods of making the same. Previously, overexpression of exogenous proteins relied on stochastic or random disposition of the exogenous proteins onto the nanovesicles for producing surface-engineered nanovesicles. This resulted in low-level, unpredictable density of the proteins of interest on nanovesicles.

Thus, in one aspect, a nanovesicle is provided comprising at least FcRn binding site wherein said FcRn binding site
(i) binds FcRn at acidic pH
(ii) lacks the ability to form homodimers; and
(ii) comprises a transmembrane domain.

The nanovesicles of the invention disclosure may be native (i.e., produced from a source cell through secretion from the endosomal, endolysomal and/or lysosomal pathway or from the plasma membrane of the source cell) nanovesicles or synthetic ones. Exemplary nanovesicles include, without being limited to, extracellular vesicles (“EVs”), microvesicles (MVs), exosomes, apoptotic bodies, ARMMs, fusosomes, microparticles and cell derived vesicular structures, membrane particles, membrane vesicles, exosome-like vesicles, ectosome-like vesicles, ectosomes or exovesicles or hybridosomes.

In one aspect, the FcRn binding polypeptides may be present on hybridosomes, i.e., hybrid biocompatible carriers which comprise structural and bioactive elements originating from EVs comprising the FcRn binding polypeptides and lipid nanoparticles comprising a tunable fusogenic moiety as described in WO2015110957. In some embodiments, isolated hybridosomes comprising FcRn binding polypeptides of the disclosure further comprise a therapeutic molecule.

The present disclosure further provides methods of producing and/or purifying nanovesicles (e.g., EVs and hybridosomes) comprising at least one polypeptide (in particular, at least one FcRn binding polypeptides) as described above. The methods may typically comprise the steps of (i) introducing into an EV-producing cell a nucleic acid which encodes the polypeptide (in particular, the FcRn binding polypeptide) as described above; and (ii) allowing for the EV-producing cell to produce EVs comprising the polypeptide (in particular, the FcRn binding polypeptide), such as cultivating the cell under suitable conditions. As a result of the of the presence of a transmembrane domain, the polypeptides (in particular, the FcRn binding polypeptides) are efficiently transported to membranes of the cell and the FcRn binding sites displayed in or on the surface of EVs. Subsequently, in step (iii), the EVs may be purified from the culture medium. Such methods may optionally comprise the step of (iv) chemically modifying the purified EVs, for example, to produce synthetic nanovesicles such as hybridosomes.

In one aspect, a method of producing nanovesicles being surface decorated with one or more FcRn binding sites is provided, comprising the steps of
(i) providing a nucleic acid or expression vector encoding a polypeptide (in particular, an FcRn binding polypeptide) as described above, comprising one or more FcRn binding sites
(ii) introducing said nucleic acid or expression vector into an EV-producing cell (e.g. mesenchymal stem cell);
(iii) cultivating said cells under suitable conditions so that EVs (e.g. exosomes) are produced; and
(iv) purifying the so produced EVs (e.g. exosomes) comprising the polypeptide (in particular, the FcRn binding polypeptide) from the cell culture.

The method may optionally comprise the step of (v) chemically modifying the EVs, for example, to produce synthetic nanovesicles such as hybrisosomes. Hybridosomes are e.g., generated by contacting the EV with a second vesicle produced in vitro, said second vesicle comprising a membrane, a fusogenic, ionizable, cationic lipid (e.g., at a molar concentration of at least 10%, at least 20%, at least 30%, at least 40%, at least 50%, and preferably at least 30% of total lipid of the second vesicle) and optionally a therapeutic agent, thereby uniting said EV with said second vesicle and producing a hybridosome.

In one aspect, a method of producing an EV comprises: a. transfecting cells with a nucleic acid described herein or an expression vector described herein; b. cultivating the cells under suitable conditions for the production of the EV; and c. collecting the EV secreted by the cells.

In one aspect, a method of producing a hybridosome comprises contacting a first EV with a second EV, thereby uniting the first EV with the second EV and producing the hybridosome, wherein said first EV has been produced in vitro, and the first EV comprises (i) a membrane, and (ii) a fusogenic, ionizable, cationic lipid (e.g., at a molar concentration of at least 10%, at least 20%, at least 30%, at least 40%, at least 50%, and preferably at least 30% of total lipid of the first EV), and wherein said second EV has been produced by a method of producing an EV described herein.

Nanovesicles (e.g., EVs and hybridosomes) comprising the polypeptides (in particular, the FcRn binding polypeptides) of the present disclosure can be produced from any type of mammalian cell that is capable of producing nanovesicles (e.g., EVs) under suitable conditions, for instance in suspension culture or in adherent culture or any other type of culturing system. Source cells as per the present disclosure may also include cells that are capable of producing nanovesicles (e.g., EVs) in vivo. The source cells may be selected from a wide range of cells and cell lines which may grow in suspension or adherent culture or be adapted to suspension growth. Generally, nanovesicles (e.g., EVs and hybridosomes) may be derived from essentially any cell source, be it a primary cell source or an immortalized cell line. The source cell may be either allogeneic, autologous, or even xenogeneic in nature to a patient to be treated, i.e. the cells may be from the patient himself or from an unrelated, matched or unmatched donor. In certain contexts, allogeneic cells may be preferable from a medical standpoint, as they could provide immuno-modulatory effects that may not be obtainable from autologous cells of a subject suffering from a certain indication. For instance, in the context of treating inflammatory or degenerative diseases, allogeneic MSCs or amnion epithelial (AE)s may be highly beneficial as nanovesicles (e.g., EV or hybridosome)-producing cell sources due to the inherent immuno-modulatory of their EVs. Cell lines of particular interest include, without being limited to, anionic fluid derived cells, induced pluripotent cells, human umbilical cord endothelial cells (HUVECs), human embryonic kidney (HEK) cells such as HEK293 cells, HEK293T cells, serum free HEK293 cells, suspension HEK293 cells, endothelial cell lines such as microvascular or lymphatic endothelial cells, erythrocytes, erythroid progenitors, chondrocytes, MSCs of different origin, amnion cells, AE cells, any cells obtained through amniocentesis or from the placenta, airway or alveolar epithelial cells, fibroblasts, endothelial cells, and epithelial cells, etc.

As described above, a source cell can be genetically modified to comprise one or more exogenous sequences (e.g., encoding one or more fusion proteins) to produce nanovesicles described herein. Preferably, the exogenous sequence encoding a polypeptide (in particular, an FcRn binding polypeptide) described herein is stably integrated into a genomic sequence of the producer cell, at a targeted site or in a random site. In some aspects, a stable cell line is generated for production of nanovesicles (e.g., EVs) comprising polypeptides (in particular, the FcRn binding polypeptides) disclosed herein. This advantageously results in consistent production of nanovesicles (e.g., EVs) of uniform quality and yield.

In some aspects, nanovesicles comprising polypeptides (in particular, the FcRn binding polypeptides) of the present disclosure can be produced from a cell transformed with a sequence encoding a full-length, scaffold protein fused to a FcRn binding site (in particular, the FcRn binding polypeptide) as disclosed herein that may additionally comprise one or more heterologous proteins (e.g. targeting domains) as described above. Any of the polypeptides (in particular, the FcRn binding polypeptides) described herein can be expressed from a plasmid, an exogenous sequence inserted into the genome or other exogenous nucleic acid, such as a synthetic messenger RNA (mRNA).

In one aspect, the present disclosure provides an EV comprising two or more interacting FcRn binding polypeptides (e.g., scaffold protein), that is produced from a cell of the present disclosure. In some embodiments, the surface density or concentration of the polypeptide (e.g., scaffold protein) on the EV described herein is increased by dimerization or oligomerization (excluding CH3-CH3 dimerization).

In some embodiments, a source cell disclosed herein is further modified to comprise an additional exogenous sequence. For example, an additional exogenous sequence can be introduced to modulate endogenous gene expression or produce a nanovesicle including a certain polypeptide as a payload. In some aspects, the source cell is modified to comprise two exogenous sequences, one encoding a polypeptide (in particular, an FcRn binding polypeptide) described herein, or a variant or a fragment thereof, and the other encoding a payload. In some aspects, the source cell is modified to comprise two exogenous sequences, one encoding a polypeptide (in particular, an FcRn binding polypeptide) described herein, or a variant or a fragment thereof, and the other encoding a polypeptide (in particular, an FcRn binding polypeptide) described herein that comprises an optional targeting moiety. In certain embodiments, the source cell can be further modified to comprise an additional exogenous sequence conferring additional functionalities to the nanovesicles (e.g., payloads, targeting moieties, or purification domains). In some aspects, the source cell is modified to comprise two exogenous sequences, one encoding a polypeptide (in particular, an FcRn binding polypeptide) disclosed herein, or a variant or a fragment thereof, and the other encoding a protein conferring the additional functionalities to nanovesicles. In some aspects, the source cell is further modified to comprise one, two, three, four, five, six, seven, eight, nine, or ten or more additional exogenous sequences.

Accordingly, the present disclosure further relates to the generation and use of EVs comprising at least one FcRn binding polypeptide wherein said FcRn binding polypetide comprises a transmembrane domain and a modified Fc domain of an immunoglobulin that (i) is capable of specifically binding to the Fc binding site of an FcRn; and (i) lacks the ability to form homodimers. In one aspect, when the FcRn binding polypeptide is expressed on nanovesicles (e.g. EVs), the transmembrane domain or a fragment thereof, to which the modified Fc domain is covalently linked (e.g. fused), provides anchorage of the modified Fc domain to the EV membrane and as a result the modified Fc domain of the FcRn binding polypeptide protrudes into the extracellular environment, which subsequently enables specific and conditional binding to the Fc binding site of FcRn.

In one aspect, nanovesicles (e.g. EVs) comprising at least one FcRn binding polypeptide of the disclosure have an increased binding affinity to the Fc binding site of an FcRn at an acidic pH and a decreased binding affinity to the Fc binding site of an FcRn at about neutral pH. In a preferred embodiment, EVs comprising at least one FcRn binding polypeptide of the disclosure have an increased propensity to form a complex with FcRn at an acidic pH as opposed to at a neutral pH.

In some embodiments the equilibrium dissociation constant of the nanovesicles (e.g. EVs.) comprising at least one FcRn binding polypeptide of the disclosure bound to FcRn at an acidic is at least 10⁻⁴, 10⁻⁵, 10⁻⁶, 10⁻⁷, 10⁻⁸ or 10⁻⁹ M. In some embodiments the equilibrium dissociation constant of the EVs comprising at least one FcRn binding polypeptide of the disclosure bound to FcRn, at an acidic pH (e.g. a pH of less than 6.5), is equal to the equilibrium dissociation constant of the modified Fc domain bound to FcRn at a pH of 6.5. In some embodiments, the equilibrium dissociation constant of the EVs comprising at least one FcRn binding polypeptide of the disclosure bound to FcRn at a pH of 6.5 is increased by at least 5%, 10%, 20%, 30%, 40%, 50% or 60% compared to the equilibrium dissociation constant of the modified Fc domain fragment bound to FcRn.

In some embodiments the equilibrium dissociation constant of the EVs comprising at least one FcRn binding polypeptide of the disclosure bound to FcRn at a neutral pH is above 10⁻⁵, 10⁻⁴, 10⁻³, 10⁻² or 10⁻¹ M. In some embodiments the equilibrium dissociation constant of the EVs comprising at least one FcRn binding polypeptide of the disclosure bound to FcRn at a neutral pH is equal to the equilibrium dissociation constant of the modified Fc domain bound to FcRn at a neutral pH. In some embodiments, the equilibrium dissociation constant of the EVs comprising at least one FcRn binding polypeptide of the disclosure bound to FcRn at a pH of 6.5 is increased by at least 20%, 30%, 40%, 50% or 60% compared to the equilibrium dissociation constant of the modified Fc domain fragment bound to FcRn.

In one embodiment of all aspects as described herein, at an acidic pH the EVs comprising at least one FcRn binding polypeptide of the disclosure has binding affinity to FcRn selected from human FcRn, cynomolgus FcRn, mouse FcRn, rat FcRn, sheep FcRn, dog FcRn and rabbit FcRn. In some embodiments, the FcRn binding polypeptide has increased binding affinity to mouse FcRn than to human FcRn.

For the EVs comprising at least one FcRn binding polypeptide of the disclosure disclosed herein, methods for analyzing binding affinity and binding kinetics are known in the art. These methods include, but are not limited to, solid-phase binding assays (e.g., ELISA assay), immunoprecipitation, surface plasmon resonance (e.g., Biacore™ (GE Healthcare, Piscataway, NJ)), kinetic exclusion assays (e.g, KinExA®), flow cytometry, fluorescence-activated cell sorting (FACS), BioLayer interferometry (e.g, Octet® (ForteBio, Inc., Menlo Park, CA)), and Western blot analysis. In some embodiments, ELISA is used to determine binding affinity. In some embodiments, surface plasmon resonance (SPR) is used to determine binding affinity and or binding kinetics. In some embodiments, kinetic exclusion assays are used to determine binding affinity and/or binding kinetics. In some embodiments, BioLayer interferometry assays are used to determine binding affinity and/or binding kinetics.

In some embodiments, the EVs comprising at least on FcRn binding polypeptide described herein may comprise modifications in the FcRn binding polypeptide that reduce or eliminate effector function. Accordingly, in some embodiments, EVs comprising at least one FcRn binding polypeptide described herein comprise modifications that reduce effector function, i.e., having a reduced ability to induce certain biological functions upon binding to an Fc receptor (other than FcRn) expressed on or in an effector cell that mediates the effector function. Examples of Fc-Receptor effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. In some embodiments, modified Fc domains present in a FcRn binding polypeptide described herein may include additional modifications that modulate effector function.

In some embodiments, the EVs comprising at least on FcRn binding polypeptide has a modified Fc domain that lacks the ability to form homodimers to avoid protein misfolding in the producer cell which can cause cytotoxicity to the protein cell, and lead to protein instability, and/or aggregation of EVs.

In another aspect, EVs comprising at least one FcRn binding polypeptides of the present disclosure may have the ability to specifically bind to antigens on specific cells in addition to binding FcRn. In some embodiments, the EVs comprising at least one FcRn binding polypeptide comprises an additional targeting domain. In some embodiments, EVs comprise FcRn binding polypeptide that may be fused to targeting domain such as an antigen-binding fragment (e.g., a Fab, Fv, or scFv) that specifically binds to an antigen. In some embodiments, an EV comprising at least one FcRn binding polypeptide contains a FcRn binding site and optionally a targeting moiety, each of which can be independently modified. In some embodiments, the modifications allow the EV comprising at least one FcRn binding polypeptide to specifically bind to a FcRn at acidic pH. The targeting moiety can be used for targeting the EV comprising at least one FcRn binding polypeptide to an antigen on specific organ, tissue, or cell.

In some aspects, the EVs comprising at least one FcRn binding polypeptide described herein demonstrate superior characteristics compared to EVs known in the art. For example, FcRn binding polypeptides comprising different transmembrane domains or fragments thereof are more enriched on the EV surface than naturally occurring EVs or the EVs produced using conventional EV proteins. In some aspects, EVs comprising FcRn binding polypeptides described herein can express greater number (e.g, 2, 3, 4, 5 or more) of FcRn binding sites, such that multiple EVs are not required. Moreover, the surface of EVs comprising FcRn binding polypeptide engineered of the present disclosure can have greater, more specific, or more controlled biological activity (e.g. targeting to specific cells or half-life) compared to naturally occurring EVs or the EVs produced using conventional transmembrane domains (e.g. Lamp2b, PTGFRN, CD63 or CD81).

In an additional aspect, FcRn binding polypeptides may be present on hybridosomes, hybrid biocompatible carriers which comprise structural and bioactive elements originating from EVs comprising the FcRn binding polypeptide and lipid nanoparticles comprising a tunable fusogenic moiety as described in WO2015110957. As a result of the presence of the FcRn binding polypeptide, the resulting hybridosomes can be isolated from unfused lipid nanoparticles by affinity chromatography methods described herein. In some embodiments, isolated hybridosomes comprising FcRn binding polypeptides of the disclosure further comprise a therapeutic agent.

### 5.4.1 Therapeutic Molecules

In some aspects, a nanovesicle (e.g., an EV or hybridosome) comprising FcRn binding polypeptides disclosed herein has been engineered or modified to deliver one or more (e.g., two, three, four, five or more) therapeutic molecules to a target cell.

The therapeutic molecule may be any inorganic or organic compound. A therapeutic molecule may decrease, suppress, attenuate, diminish, arrest, or stabilize the development or progression of disease, disorder, or cell growth in an animal such as a mammal or human. Examples of therapeutic molecule that can be introduced into a nanovesicle (e.g., an EV or hybridosome) comprising FcRn binding polypeptides include therapeutic agents such as, nucleic acids (e.g., DNA or mRNA molecules that encode a polypeptide such as an enzyme, mRNA molecules that encode a polypeptide such as an antigen or RNA molecules that have regulatory function such as miRNA, dsDNA, and lncRNA), amino acids (e.g., amino acids comprising a detectable moiety or a toxin or that disrupt translation), polypeptides (e.g., enzymes, enzymes for gene editing, nucleic acid binding proteins, antibodies, intrabodies, single chain variable fragments (scFv), affibodies, bi- and multispecific antibodies or binders, affibodies, darpins, receptors, ligands, or fragments thereof), lipids, carbohydrates, and small molecules (e.g., small molecule drugs and toxins). In certain embodiments, the therapeutic molecules may be a substance used in the diagnosis, treatment, or prevention of a disease or as a component of a medication. In some embodiments, a payload may refer to a compound that facilitates obtaining diagnostic information about a targeted site in a body of a living organism, such as a mammal or in a human. For example, imaging agents may be classified as active agents in the present disclosure as they are substances that provide imaging information required for diagnosis.

Further non-limiting examples of therapeutic nucleic acids intended to be used in the present disclosure are siRNA, small or short hairpin RNA (shRNA), guide RNA (gRNA), single guide RNA (sgRNA), clustered regularly interspaced short palindromic repeat RNA (crRNA), trans-activating clustered regularly interspaced short palindromic repeat RNA (tracrRNA) immune-stimulating oligonucleotides, plasmids, antisense nucleic acids and ribozymes. In certain embodiments the therapeutic nucleic acid may be DNA, including linear DNA, circular DNA, or an artificial chromosome. In some embodiments the therapeutic DNA is maintained episomally. In some embodiments the therapeutic DNA is integrated into the genome. The therapeutic RNA may be chemically modified RNA, e.g., may comprise one or more backbone modification, sugar modifications, noncanonical bases, or caps. Backbone modifications include, e.g., phosphorothioate, N3′ phosphoramidite, boranophosphate, phosphonoacetate, thio-PACE, morpholino phosphoramidites, or PNA. Sugar modifications include, e.g., 2′-0-Me, LNA, UNA, and 2′-0-MOE. Noncanonical bases include, e.g., 5-bromo-U, and 5-iodo-U, 2,6-diaminopurine, C-5 propynyl pyrimidine, difluorotoluene, difluorobenzene, dichlorobenzene, 2-thiouridine, pseudouridine, and dihydrouridine. Caps include, e.g., ARCA. Additional modifications are discussed, e.g., in Deleavey et al., “Designing Chemically Modified Oligonucleotides for Targeted Gene Silencing” Chemistry & Biology Volume 19, Issue 8, 24 Aug. 2012, Pages 937-954.

Non-limiting examples of other suitable therapeutic molecules include pharmacologically active drugs and genetically active molecules, including antineoplastic agents, anti-inflammatory agents, hormones or hormone antagonists, ion channel modifiers, and neuroactive agents. Examples of suitable payloads of therapeutic agents include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Suitable payloads further include toxins, and biological and chemical warfare agents, for example see Somani, S. M. (ed.), Chemical Warfare Agents, Academic Press, New York (1992)).

In one aspect, the nanovesicles comprising a scaffold protein and a modified Fc domain can bestow several desirable properties upon the nanovesicle including increased serum half-life, shorter blood clearance and improved affinity purification. In some embodiments, the nanovesicles described herein can be modified to increase or decrease their half-life in circulation. In some embodiments, the half-life of the therapeutic cargo in the nanovesicle comprising the polypeptide described herein in circulation can be modified by altering the half-life of the nanovesicle. In some instances, the half-life is increased and the increase can be, for instance from about 1.5-fold to 20-fold for a therapeutic agent payload maintained in the nanovesicle comprising polypeptides described herein when compared to a therapeutic agent not contained in the nanovesicle and the half-life being measured in a serum-containing solution.

In certain embodiments, presence or absence of the nanovesicle and/or the therapeutic molecule payload in the circulatory system, is determined by the presence or absence of certain polypeptides or fragments thereof on the nanovesicle, for example, a modified Fc domain polypeptide or a functional fragment thereof.

In some embodiments, the nanovesicles comprising the polypeptides described herein are capable of being present in the circulatory system or tissue of a subject for an extended period of time, allowing the delivery of a more efficient therapeutic effect than what can be achieved by nanovesicles devoid of said polypeptides. Half-life extension is a particular advantage when compared to current EV-based therapies not involving scaffold proteins comprising modified Fc domains.

Effective amounts of scaffold proteins comprising modified Fc domains include 1, 2, 3, 4, 5, 6, 7, 8, 9, 10, 20, 30, 40, 60, 80, 100 or more polypeptides per nanovesicle. Alternatively, an effective amount is the amount capable of extending the nanovesicle half-life by 10%, 20%, 30%, 40%, 50%, 60%, 70%, 80%, 90%, 100%, 200%, 400%, 800%, 1,000%, or 10,000% relative to the half-life that the nanovesicle would exhibit without the polypeptides.

In some embodiments, the nanovesicles (e.g., EVs or hybridosomes) described herein have properties that can be demonstrated with the following methods. In some embodiments, contents of said nanovesicles can be extracted for study and characterization. In some embodiments, nanovesicles are isolated and characterized by metrics including, but not limited to, size, shape, morphology, or molecular compositions such as nucleic acids, proteins, metabolites, and lipids as well as half-life and pharmacodynamics.

In some embodiments, the methods described herein comprise measuring the size of nanovesicle and/or populations of nanovesicle included in the purified fractions. In some embodiments, nanovesicle size is measured as the longest measurable dimension. Generally, the longest general dimension of an nanovesicle is also referred to as its diameter.

Nanovesicle size can be measured using various methods known in the art, for example, nanoparticle tracking analysis, multi-angle light scattering, single angle light scattering, size exclusion chromatography, analytical ultracentrifugation, field flow fractionation, laser diffraction, tunable resistive pulse sensing, or dynamic light scattering.

In some embodiments, the methods described herein comprise measuring the density of FcRn biding polypeptides on the nanovesicle surface. The surface density can be calculated or presented as the mass per unit area, the number of proteins per area, number of molecules or intensity of molecule signal per nanovesicle, molar amount of the protein, etc. The surface density can be experimentally measured by methods known in the art, for example, by using bio-layer interferometry (BLI), FACS, Western blotting, fluorescence (e.g., GFP-fusion protein) detection, nano-flow cytometry, ELISA, alphaLISA, and/or densitometry by measuring bands on a protein gel.

### 5.5 Purification of Nanovesicles Comprising FcRn Binding Polypeptides

The use of nanovesicles for medical purposes requires that the nanovesicles be free or mostly free of impurities in the culture supernatant including but not limited to macromolecules, such as nucleic acids, contaminant proteins, lipids, carbohydrates, metabolites, small molecules, metals, or a combination thereof. The present disclosure provides a method of purifying nanovesicles comprising a FcRn binding polypeptide from contaminating macromolecules. In some embodiments, purified nanovesicles comprising a FcRn binding polypeptide are substantially free of contaminating macromolecules.

In some cases, isolation, purification and removal of nanovesicles comprising FcRn binding polypeptides are done by column chromatography using a column where the FcRn and the solid support (e.g. a resin) are packed within the column. In some embodiments, a sample containing nanovesicles comprising the FcRn binding polypeptide of the disclosure is loaded and run through the column to allow binding, optionally, a wash buffer is run through the column, and the elution buffer is subsequently applied to the column and the eluate containing the nanovesicles comprising the FcRn binding polypeptide of the disclosure is collected. These steps can be done at ambient pressure or with application of additional pressure. In some cases, isolation, purification, and elution of nanovesicles comprising the FcRn binding polypeptide are done using a batch treatment. For example, a sample is added to the FcRn attached to a solid support in a vessel, followed by mixing, separating the solid support, subsequent removing the liquid phase, washing, centrifuging, adding the elution buffer, re-centrifuging and removing the elute. In some cases, a hybrid method can be employed. For example, a sample is added to the FcRn attached to a solid support in a vessel, the solid support bound to the nanovesicles comprising FcRn binding polypeptides is subsequently packed onto a column, and washing and elution are done on the column.

Generally speaking, the affinity purification methods of the nanovesicles comprising FcRn binding polypeptides of the present disclosure will result in a pure, highly enriched nanovesicle population. However, additional isolation, purification, and/or polishing steps may be included both upstream and/or downstream of the affinity purification step. Suitable complementary purification steps include size exclusion liquid chromatography, bead-elute liquid chromatography, ionic exchange purification (such as anionic exchange), charged membrane separation, and various other purification and/or polishing strategies used in the art.

In some embodiments, a nanovesicle comprising FcRn binding polypeptides sample is isolated or purified with a FcRn binding agent is subsequently processed with a different binding agent (e.g. protein affinity binder, ion exchange or mixed mode resin). In some embodiments, more than one columns are used in series, where each of the multiple columns contains a different binding agent specific to a different target protein. In some embodiments, a single column contains multiple binding agents, each specific to a different target protein.

Also provided herein is a method for separating nanovesicles comprising FcRn binding polypeptides from nanovesicles not comprising said polypeptides (e.g. non-surface decorated EVs or lipid nanoparticles). In specific embodiments, the subpopulation of nanovesicles comprising the FcRn binding polypeptides is distinguished from other subpopulations by forming a complex with a FcRn at acidic pH.

In one embodiment the chromatography material comprising a FcRn as ligand has a stability of at least 3 cycles in the methods and uses as described herein. A cycle is a pH gradient from the first pH value to the second pH value of the respective method. Thus, in one embodiment a cycle is a pH gradient from about pH value pH 5.5 to about pH value pH 8.8.

Once at least one eluted fraction is collected, a composition of the eluted fraction can be analyzed. For example, the concentration of nanovesicles comprising FcRn binding polypeptide, a host cell protein, a contaminant protein, DNA, carbohydrates, or lipids can be measured in each eluted fraction. Other properties of nanovesicles in each eluted fraction can be also measured. The properties include an average size, an average charge density, and other physiological properties related to bio-distribution, cellular uptake, half-life, pharmacodynamics, potency, dosing, immune response, loading efficiency, stability, or reactivity to other compounds.

In some aspects, nanovesicles comprising FcRn binding polypeptide variants with increased affinity for the FcRn (i.e. increased retention time on an FcRn column but still eluting at a pH value below pH 7.4 as described herein compared to a native nanovesicle) may be predicted to have longer serum half-lives compared to those with decreased affinity for the FcRn. Nanovesicles comprising FcRn binding polypeptide variants with increased affinity for the FcRn have applications in methods of treating mammals, especially humans, where long half-life of the administered EV is desired, such as in the treatment of a chronic disease or disorder.

Some embodiments of the present invention relate to isolation, purification and sub-fractionation of nanovesicles using a specific binding interaction (i.e. affinity purification) between a FcRn binding polypeptide (e.g. a scaffold protein of the disclosure linked to polypeptide comprising a FcRn binding site) enriched on the nanovesicle membrane and an immobilized binding agent (e.g. a FcRn). These methods generally comprise the steps of (1) applying or loading a sample comprising nanovesicles of the disclosure to the immobilized agent, (2) optionally washing away unbound sample components using appropriate buffers that maintain the binding interaction between the FcRn binding polypeptide displayed on nanovesicles and binding agents, and (3) eluting (dissociating and recovering) the nanovesicles from the immobilized binding agents by altering the buffer conditions so that the binding interaction no longer occurs.

In some aspects, the affinity purification method to purify nanovesicles comprising at least one polypeptide (in particular, at least one FcRn binding polypeptides) described herein has superior recovery yields compared to other affinity purification of nanovesicles known in the art. For example, nanovesicles comprising at least one polypeptide (in particular, at least one FcRn binding polypeptides) described herein can be eluted from the immobilized binding partner at a mild pH (e.g. pH 7-pH 9) compared to conventional affinity purification methods requiring a pH of less than 5 sometimes less than pH of 3 to elute (e.g. dissociate) the nanovesicles from the immobilized binding partner (e.g. protein A).

The use of nanovesicles (e.g., EVs or hybridosomes) for medical purposes furthermore requires that the nanovesicles (e.g., EVs or hybridosomes) are not in an aggregated form and exhibit colloidal stability however, a very acidic pH can cause colloidal instabilities. An important aspect of the present disclosure is to provides methods of purifying nanovesicles (e.g., EVs or hybridosomes) comprising at least one FcRn binding polypeptides at more physiological conditions, such as a physiological pH value.

Some aspects of the present disclosure relate to isolation and purification of nanovesicles (e.g., EVs or hybridosomes) comprising FcRn binding polypeptides using a specific binding interaction between a first binding partner (e.g. a FcRn binding polypeptide present on the nanovesicle membrane) and a second binding partner (e.g. an immobilized FcRn). These methods generally comprise the steps of (1) applying or loading a sample comprising nanovesicles comprising the first binding partner (e.g. a FcRn binding polypeptide present on the nanovesicle membrane) onto a matrix containing the second binding partner (e.g. an immobilized FcRn), (2) optionally washing away unbound sample components using appropriate buffers that maintain the binding interaction between the first (e.g. a FcRn binding polypeptide present on the nanovesicle membrane) and second binding partner (e.g. an immobilized FcRn), and (3) eluting (dissociating and recovering) the nanovesicles comprising FcRn binding polypeptides from the immobilized binding FcRn agents by altering the buffer conditions so that the binding interaction between the binding partners no longer occurs.

In some embodiments, the second binding partner is a FcRn which is optionally immobilized on an appropriate matrix or chromatography material. In some embodiments, the second binding partner used for this isolation and purification process, is a FcRn protein produced in vitro by a producer cell by a genetic modification or transfection, or an isolated FcRn protein modified by chemical, physical or other biological methods. In some cases, the FcRn protein is a non-mutant FcRn protein or a mutant FcRn protein, e.g., a variant or a fragment of an FcRn protein. In some cases, the FcRn is a fusion protein. In a specific embodiment, the FcRn is a soluble single-chain FcRn as generated following the methods of Feng et al. (2011), Protein Expr. Purif. 79:66-71. In one embodiment the soluble FcRn forms a non-covalent heterodimer with beta-2-microglobulin (B2M).

In specific embodiments, provided herein is a method for purifying an EV, wherein said method comprises: a. providing the EV wherein the EV is associated with a first binding partner, wherein the first binding partner is capable of binding to the Fc binding site of an FcRn in a pH dependent manner; b. contacting at a first pH the EV associated with the first binding partner with a second binding partner, wherein the second binding partner comprises the Fc binding site of the FcRn and is associated with a solid matrix; and c. eluting the EV associated with the first binding partner from the solid matrix at a second pH. In certain embodiments, the method further comprises a washing step at the first pH. In certain embodiments, the first pH is below 6.5. In certain embodiments, the second pH is above 7.4.

In specific embodiments, provided herein is a method for purifying an EV, wherein said method comprises: a. providing the EV wherein the EV is associated with a first binding partner, wherein the first binding partner is capable of binding to the Fc binding site of an FcRn in a pH dependent manner and comprises or consists of a polypeptide described herein; b. contacting at a first pH the EV associated with the first binding partner with a second binding partner, wherein the second binding partner comprises the Fc binding site of the FcRn and is associated with a solid matrix; and c. eluting the EV associated with the first binding partner from the solid matrix at a second pH. In certain embodiments, the method further comprises a washing step at the first pH. In certain embodiments, the first pH is below 6.5. In certain embodiments, the second pH is above 7.4.

In specific embodiments, provided herein is a method for purifying a hybridosome, wherein said method comprises: a. providing the hybridosome wherein the hybridosome is associated with a first binding partner, wherein the first binding partner is capable of binding to the Fc binding site of an FcRn in a pH dependent manner; b. contacting at a first pH the hybridosome associated with the first binding partner with a second binding partner, wherein the second binding partner comprises the Fc binding site of the FcRn and is associated with a solid matrix; and c. eluting the hybridosome associated with the first binding partner from the solid matrix at a second pH. In certain embodiments, the method further comprises a washing step at the first pH. In certain embodiments, the first pH is below 6.5. In certain embodiments, the second pH is above 7.4.

In specific embodiments, provided herein is a method for purifying an hybridosome, wherein said method comprises: a. providing the hybridosome wherein the hybridosome is associated with a first binding partner, wherein the first binding partner is capable of binding to the Fc binding site of an FcRn in a pH dependent manner and comprises or consists of a polypeptide described herein; b. contacting at a first pH the hybridosome associated with the first binding partner with a second binding partner, wherein the second binding partner comprises the Fc binding site of the FcRn and is associated with a solid matrix; and c. eluting the hybridosome associated with the first binding partner from the solid matrix at a second pH. In certain embodiments, the method further comprises a washing step at the first pH. In certain embodiments, the first pH is below 6.5. In certain embodiments, the second pH is above 7.4. In one embodiment of all aspects as described herein, the FcRn is selected from human FcRn, cynomolgus FcRn, mouse FcRn, rat FcRn, sheep FcRn, dog FcRn and rabbit FcRn.

In one embodiment of all aspects as described herein the beta-2-microglobulin is selected from human beta-2-microglobulin, cynomolgus beta-2-microglobulin, mouse beta-2-microglobulin, rat beta-2-microglobulin, sheep beta-2-microglobulin, dog beta-2-microglobulin and rabbit beta-2-microglobulin.

In one embodiment, the hetero dimer is composed of beta-2-microglobulin and soluble FcRn from the same species. In one embodiment, the hetero dimer is composed of beta-2-microglobulin and soluble FcRn from the different species.

Thus, a chromatography material comprising a complex of neonatal Fc receptor (FcRn) and beta-2-microglobulin as ligand as described herein can be used for the isolation/separation of extracellular vesicles displaying monomeric FC and, thus, provides for an alternative to conventional Protein A affinity chromatography. In addition, by using the chromatography material as described herein, the separation can be effected at more physiological conditions, such as pH value, compared to conventional Protein A affinity chromatography.

Methods to prepare soluble and functional FcRn are known in the art. One method includes expressing in mammalian cells soluble human FcRn (sFcRn) as a single-chain soluble fusion protein (see SEQ ID NOs: 70 and 71 for the amino acid sequences of human single-chain FcRn and mouse single-chain FcRn, respectively). The highly hydrophilic beta-2-microglobulin is joined with the hydrophobic heavy chain via a 15 amino acid linker. The single-chain fusion protein format improves the expression level of the heavy chain but also simplified the purification process (Feng et al. (2011), Protein Expr. Purif. 79:66-71). The use of an immobilized non-covalent complex of a FcRn and beta-2-microglobulin (b2m) as affinity chromatography ligand in an affinity chromatography for soluble Fc-fusion proteins with a positive linear pH gradient is described in WO2013/120929. Recombinant FcRn and variants thereof for purification of Fc-containing soluble fusion proteins is described in WO 2010/048313.

In one embodiment, the second binding agent comprises the soluble extracellular domain of FcRn (e.g. SEQ ID NO: 68 for human FcRn) with C-terminal His-Avi Tag or C-Tag co-expressed with β₂-microglobulin (SEQ ID NO: 69 for human beta-2-microglobulin) in mammalian cells. In some embodiments, the non-covalent or single chain FcRn-complex is biotinylated and loaded onto streptavidin derivatized sepharose. In some embodiments, the non-covalent FcRn-complex comprising a c-tag is loaded onto C-TagX1 beads.

| Seq | Descrip- | |
|---|---|---|
| ID | tion | Sequence |
| 68 | Soluble | SAESHLSLLYHLTAVSSPAPGTPAFWVSGWLGPQQY |
| | hFcRn | LSYNSLRGEAEPCGAWVWENQVSWYWEKETTDLRI |
| | | KEKLFLEAFKALGGKGPYTLQGLLGCELGPDNTSVP |
| | | TAKFALNGEEFMNFDLKQGTWGGDWPEALAISQR |
| | | WQQQDKAANKELTFLLFSCPHRLREHLERGRGNLE |
| | | WKEPPSMRLKARPSSPGFSVLTCSAFSFYPPELQLRF |
| | | LRNGLAAGTGQGDFGPNSDGSFHASSSLTVKSGDEH |
| | | HYCCIVQHAGLAQPLRVELESPAK |
| 69 | Soluble | METDTLLLWVLLLWVPGSTGDAAQPARRAVRSLVP |
| | b2m | SSGLEAIQRTPKIQVYSRHPAENGKSNFLNCYVSGFH |
| | | PSDIEVDLLKNGERIEKVEHSDLSFSKDWSFYLLYYT |
| | | EFTPTEKDEYACRVNHVTLSQPKIVKWDRDM |
| 70 | Human | METDTLLLWVLLLWVPGSTGDAAQPARRAVRSLVP |
| | scFcRn | SSGLEAIQRTPKIQVYSRHPAENGKSNFLNCYVSGFH |
| | | PSDIEVDLLKNGERIEKVEHSDLSFSKDWSFYLLYYT |
| | | EFTPTEKDEYACRVNHVTLSQPKIVKWDRDMGGGG |
| | | SGGGGSGGGGSAESHLSLLYHLTAVSSPAPGTPAFW |
| | | VSGWLGPQQYLSYNSLRGEAEPCGAWVWENQVSW |
| | | YWEKETTDLRIKEKLFLEAFKALGGKGPYTLQGLLG |
| | | CELGPDNTSVPTAKFALNGEEFMNFDLKQGTWGGD |
| | | WPEALAISQRWQQQDKAANKELTFLLFSCPHRLREH |
| | | LERGRGNLEWKEPPSMRLKARPSSPGFSVLTCSAFSF |
| | | YPPELQLRFLRNGLAAGTGQGDFGPNSDGSFHASSS |
| | | LTVKSGDEHHYCCIVQHAGLAQPLRVELESPAK |
| 71 | Mouse | METDTLLLWVLLLWVPGSTGDAAQPARRAVRSLVP |
| | scFcRn | SSGLYAIQKTPQIQVYSRHPPENGKPNILNCYVTQFH |
| | | PPHIEIQMLENGKKIPKVEMSDMSFSKDWSFYILAHT |
| | | EFTPTETDTYACRVKHASMAEPKTVYWDRDMGGG |
| | | GSGGGGSGGGGSRPPLMYHLTAVSNPSTGLPSFWAT |
| | | GWLGPQQYLTYNSLRQEADPCGAWMWENQVSWY |
| | | WEKETTDLKSKEQLFLEALKTLEKILNGTYTLQGLL |
| | | GCELASDNSSVPTAVFALNGEEFMKFNPRIGNWTGE |
| | | WPETEIVANLWMKQPDAARKESEFLLNSCPERLLGH |
| | | LERGRRNLEWKEPPSMRLKARPGNSGSSVLTCAAFS |
| | | FYPPELKFRFLRNGLASGSGNCSTGPNGDGSFHAWS |
| | | LLEVKRGDEHHYQCQVEHEGLAQPLTVDLDSSARSS |

An exemplary affinity chromatography column comprises a matrix and matrix bound chromatographical functional groups, characterized in that the matrix bound chromatographical functional group comprises a complex of neonatal Fc receptor (FcRn) and beta-2-microglobulin. A further exemplary affinity chromatography column comprises a matrix and matrix bound chromatographical functional groups, characterized in that the matrix bound chromatographical functional group comprises a single chain fusion protein of a soluble neonatal Fc receptor (FcRn) and beta-2-microglobulin.

In a preferred embodiment, the FcRn is attached to a solid phase, to enable e.g. chromatography and/or membrane-based purification. Affinity chromatography is generally based on the highly selective interaction between an immobilized FcRn ligand and a structural element on the target biomolecule (e.g. FcRn binding site on the FcRn binding polypeptide). In one embodiment, said target biomolecule is a FcRn binding polypeptide of the disclosure and the structural element is a FcRn binding site (e.g. a modified Fc domain). In another embodiment, said target biomolecule is a nanovesicle comprising the FcRn binding polypeptide of the disclosure and the structural element is the modified Fc domain. The high selectivity of affinity chromatography may be provided by multiple molecular interactions (including hydrogen bonds, hydrophobic interactions, ionic interactions and/or van der Waals interactions) between the FcRn immobilized on an appropriate matrix (e.g. the chromatography matrix) and the modified Fc domain forming part of the FcRn binding polypeptide of the present disclosure. Suitable second binding partner for affinity-based purification of the FcRn binding polypeptide-containing nanovesicles, is a soluble heterodimer of the FcRn receptor and any combination, derivative, domain or part thereof.

In a further aspect, a chromatography method comprising FcRn as a second binding partner as described herein can be used for the isolation/separation of nanovesicles displaying FcRn binding polypeptides and, thus, provides for an alternative to conventional Protein A affinity chromatography. In addition, by using the chromatography material as described herein the separation can be effected at more physiological conditions, such as pH value, compared to conventional Protein A affinity chromatography.

In some embodiments, isolating or purifying as used herein is the process of removing, partially removing (e.g., removing a fraction) of the FcRn binding polypeptide or nanovesicles comprising said FcRn binding polypeptide from a sample containing producer cells. In some embodiments, isolating or purifying as used herein is the process of removing, partially removing (e.g., removing a fraction) of the FcRn binding polypeptide or nanovesicles (e.g. EVs) comprising said FcRn binding polypeptide from a sample after removal of producer cells (e.g. after removal of cells by centrifugation or depth filtration).

The FcRn can be chemically immobilized or coupled (e.g. via biotin-streptavidin binding) to a solid support so that the population of nanovesicle comprising FcRn binding polypeptides have specific affinity to the FcRn, become bound at acidic pH. Various forms of solid support can be used, e.g., a porous agarose bead, a microtiter plate, a magnetic bead, or a membrane. In some embodiments, the solid support forms a chromatography column and can be used for affinity chromatography of nanovesicles comprising FcRn binding polypeptides.

A “solid support” denotes a non-fluid substance, and includes particles (including microparticles and beads) made from materials such as polymer, metal (paramagnetic, ferromagnetic particles), glass, and ceramic; gel substances such as silica, alumina, and polymer gels; capillaries, which may be made of polymer, metal, glass, and/or ceramic; zeolites and other porous substances; electrodes; microtiter plates; solid strips; and cuvettes, tubes or other spectrometer sample containers. A solid phase component of an assay is distinguished from inert solid surfaces in that a solid support contains at least one moiety on its surface, which is intended to interact chemically with a molecule. A solid phase may be a stationary component, such as a chip, tube, strip, cuvette, or microtiter plate, or may be non-stationary components, such as beads and microparticles. Microparticles can also be used as a solid support for homogeneous assay formats. A variety of microparticles that allow both non-covalent or covalent attachment of FcRn complex and other substances may be used. In one embodiment the solid support is composed of POROS™ beads.

The conjugation of the FcRn to the solid support can be performed by chemically binding via N-terminal and/or F-amino groups (lysine), F-amino groups of different lysins, carboxy-, sulfhydryl-, hydroxyl-, and/or phenolic functional groups of the amino acid backbone of the protein, and/or sugar alcohol groups of the carbohydrate structure of the protein.

### 5.5.1 Binding

Some aspects of the present disclosure relate to isolation and purification of nanovesicles comprising FcRn binding polypeptides using a specific binding interaction between a first binding partner (e.g. a FcRn binding polypeptide present on the nanovesicle membrane) and a second binding partner (e.g. an immobilized FcRn).

In one embodiment, a sample containing FcRn binding polypeptide of the disclosure or nanovesicles (e.g., EVs or hybridosomes) comprising said polypeptide is adjusted to a first pH (i.e. acidic pH) and then applied to the FcRn affinity column. In one embodiment, the production mixture or the crude or the partly purified cultivation supernatant containing the FcRn binding polypeptide of the disclosure is adjusted to a first pH (i.e. acidic pH) and then applied to the FcRn affinity column. In one embodiment the first pH is below about pH 6.5. In a preferred embodiment the first pH is below about pH 6.5 and above pH 5.

In one embodiment the first pH value is about pH 5 to about pH 6. In one embodiment the first pH value is about pH 5 or about pH 5.5 or about pH 6. In one embodiment the first pH value is selected from about pH 3.5, about pH 3.6, about pH 3.7, about pH 3.8, about pH 3.9, about pH 4.0, about pH 4.1, about pH 4.2, about pH 4.3, about pH 4.4, about pH 4.5, about pH 4.6, about pH 4.7, about pH 4.8, about pH 4.9, about pH 5.0, about pH 5.1, about pH 5.2, about pH 5.3, about pH 5.4, about pH 5.5, about pH 5.6, about pH 5.7, about pH 5.8, about pH 5.9, about pH 6.0, about pH 6.1, about pH 6.2, about pH 6.3, and about pH 6.4.

The methods described herein requires specific interaction between a FcRn binding polypeptide and a FcRn purification ligand. High-throughput screening can be performed to identify further buffer conditions ideal for the specific binding—mainly through altering pH but optionally also salt concentration and/or reducing polarity with an organic modifier, ethylene glycol, propylene glycol, or urea. The interaction between the FcRn binding polypeptide of the disclosure and a binding agent (e.g. FcRn) can also change depending on sample conditions (e.g., sample amount loaded per volume of chromatographic resin, concentration of FcRn binding polypeptide, concentration of EVs comprising FcRn binding polypeptide, concentration of impurities), loading buffers (e.g., pH, salt concentrations, salt identity, polarity), and other physical conditions (e.g., temperature). In addition, residence time can be adjusted based on differential adsorption rates between impurities and FcRn binding polypeptides or nanovesicles comprising said polypeptides. Thus, various purification conditions described herein can be tested to identify ideal conditions for the step.

Similar approaches can be used to improve purity and yield, and aid in enriching, depleting, or isolating sub-populations of nanovesicles comprising FcRn binding polypeptides. These properties, along with maximizing load challenge and applying more stringent elution conditions, could be employed to further enhance the concentration of exosomes.

### 5.5.2 Elution

In one aspect, the recovering of FcRn binding polypeptides of the disclosure or nanovesicles (e.g., EVs or hybridosomes) comprising said FcRn binding polypeptides, bound to the FcRn affinity column in the uses and methods as described herein, is primarily by changing the pH of the buffered solution from a first pH value (i.e. more acidic pH) that favors binding, to a second pH value (i.e. less acidic, more neutral or more alkaline) in which binding between the binding pair is less favorable.

In some embodiments, elution is favored by a change of pH that is done by a positive linear pH gradient which denotes a pH gradient starting at a low first pH value (i.e. more acidic pH value) and ending at a second higher pH value (i.e. less acidic, a neutral or alkaline pH value), wherein the pH of the eluent is changed continuously as a function of time. An example of a continuous pH gradient is a linear pH gradient, wherein the change in pH is a linear function of time. In one embodiment, a continuous pH gradient can be established by utilizing two or more buffers of differing pH which are mixed together to form the eluent. The ratio of the buffers within the eluent, and, thus, the pH of the eluent, can thus be varied continuously as a function of time. Control of the buffer mixing process is typically controlled by a flow controller, which is programmed to produce the desired pH gradient. In one embodiment the positive linear pH gradient starts at a first pH value of about 5.5 and ends at a second pH value of about 8.8.

In some embodiments, change of pH is achieved by a positive step pH gradient starting at a low (i.e. more acidic pH value) and ending at a higher (i.e. less acidic, neutral or alkaline pH value), wherein the change in pH is discontinuous with respect to time, forming one or more steps, or time points wherein the pH undergoes an abrupt change. This can be accomplished simply by replacing as eluent a first buffer with a second buffer of different pH. In a preferred embodiment of the method the gradient employed is a step pH gradient. In one embodiment the positive step pH gradient starts at a first pH value of about 5.5 and ends at a second pH value of about 8.8.

In one embodiment the second pH value is about pH 7.3 to about pH 9.5. In one embodiment the second pH value is about pH 8.5 to about pH 9. In one embodiment the second pH value is about pH 8.8.

In one embodiment the second pH value is selected from about pH 7.1, about pH 7.2, about pH 7.3, about pH 7.4, about pH 7.5, about pH 7.6, about pH 7.7, about pH 7.8., about pH 7.9, about pH 8.0, about pH 8.0, about pH 8.1, about pH 8.2, about pH 8.3, about pH 8.4, about pH 8.5, about pH 8.6, about pH 8.7, about pH 8.8, about pH 8.9, about pH 9.0, about pH 9.1, about pH 9.2, about pH 9.3, about pH 9.4, and about pH 9.5.

In one embodiment, the first pH value is about pH 3.5, about pH 3.6, about pH 3.7, about pH 3.8, about pH 3.9, about pH 4.0, about pH 4.1, about pH 4.2, about pH 4.3, about pH 4.4, about pH 4.5, about pH 4.6, about pH 4.7, about pH 4.8, about pH 4.9, about pH 5.0, about pH 5.1, about pH 5.2, about pH 5.3, about pH 5.4, about pH 5.5, about pH 5.6, about pH 5.7, about pH 5.8, about pH 5.9, about pH 6.0, about pH 6.1, about pH 6.2, about pH 6.3, or about pH 6.4, and the second pH value is about 7.1, about pH 7.2, about pH 7.3, about pH 7.4, about pH 7.5, about pH 7.6, about pH 7.7, about pH 7.8., about pH 7.9, about pH 8.0, about pH 8.0, about pH 8.1, about pH 8.2, about pH 8.3, about pH 8.4, about pH 8.5, about pH 8.6, about pH 8.7, about pH 8.8, about pH 8.9, about pH 9.0, about pH 9.1, about pH 9.2, about pH 9.3, about pH 9.4, or about pH 9.5.

In some embodiments, elution of FcRn bound nanovesicles comprising FcRn binding polypeptides can be alternatively achieved through altering salt concentration, and/or polarity with an organic modifier, ethylene glycol, propylene glycol, or urea.

In some embodiments, aside from modulating the pH range, elution can also be achieved by modulating, salts, organic solvents, small molecules, detergents, zwitterions, amino acids, polymers, temperature, and any combination of the above. Similar elution agents can be used to improve purity, improve yield, and isolate sub-populations of nanovesicles comprising FcRn binding polypeptides.

In some embodiments, elution can be also done with multiple elution buffers having different properties, such as pH, salts, organic solvents, small molecules, detergents, zwitterions, amino acids, polymers, temperature, and any combination of the above. A plurality of eluted fractions can be collected, wherein nanovesicles comprising FcRn binding polypeptides collected in each fraction has different properties. For example, nanovesicles comprising FcRn binding polypeptides collected in one fraction has a higher purity, a smaller or larger average size, or a preferred composition, etc. than FcRn binding nanovesicles in other fractions.

In principle any buffer substance can be used in the methods as described herein. In one embodiment a pharmaceutically acceptable buffer substance is used, such as e.g. phosphoric acid or salts thereof, acetic acid or salts thereof, citric acid or salts thereof, morpholine, 2-(N-morpholino) ethanesulfonic acid (MES) or salts thereof, histidine or salts thereof, glycine or salts thereof, tris (hydroxymethyl) aminomethane (TRIS) or salts thereof, (4-(2-hydroxyethyl)-1-piperazineethanesulfonic acid (HEPES) or salts thereof.

One specific embodiment relates to a method of removing non-decorated nanovesicles from a sample using a specific binding interaction between FcRn binding polypeptide and an immobilized FcRn binding agent. In these cases, nanovesicles bound to the binding agent are not eluted from the binding agent and a fraction which does not bind to the binding agent can be collected.

Selective elution of FcRn binding polypeptide or nanovesicles comprising said polypeptide can be achieved by increasing the concentration of a monovalent cationic halide salt (e.g., sodium chloride, potassium chloride, sodium bromide, lithium chloride, sodium iodide, potassium bromide, lithium bromide, sodium fluoride, potassium fluoride, lithium fluoride, lithium iodide, sodium acetate, potassium acetate, lithium acetate, and potassium iodide), a divalent or trivalent salt (e.g., calcium chloride, magnesium chloride, calcium sulfate, sodium sulfate, magnesium sulfate, chromium trichloride, chromium sulfate, sodium citrate, iron (III) chloride, yttrium (III) chloride, potassium phosphate, potassium sulfate, sodium phosphate, ferrous chloride, calcium citrate, magnesium phosphate, and ferric chloride), or a combination thereof, in the elution buffer, through the use of an increasing gradient (step or linear) of a monovalent cationic halide salt (e.g., sodium chloride, potassium chloride, sodium bromide, lithium chloride, sodium iodide, potassium bromide, lithium bromide, sodium fluoride, potassium fluoride, lithium fluoride, lithium iodide, sodium acetate, potassium acetate, lithium acetate, and potassium iodide), a divalent or trivalent salt (e.g., calcium chloride, magnesium chloride, calcium sulfate, sodium sulfate, magnesium sulfate, chromium trichloride, chromium sulfate, sodium citrate, iron (III) chloride, yttrium (III) chloride, potassium phosphate, potassium sulfate, sodium phosphate, ferrous chloride, calcium citrate, magnesium phosphate, and ferric chloride), or a combination thereof, at a fixed pH.

In one embodiment the buffer substance is selected from phosphoric acid or salts thereof, or acetic acid or salts thereof, or citric acid or salts thereof, or histidine or salts thereof.

In one embodiment the buffer substance has a concentration of from 5 mM to 500 mM. In one embodiment the buffer substance has a concentration of from 10 mM to 300 mM. In one embodiment the buffer substance has a concentration of from 10 mM to 250 mM. In one embodiment the buffer substance has a concentration of from 10 mM to 100 mM. In one embodiment the buffer substance has a concentration of from 15 mM to 50 mM. In one embodiment the buffer substance has a concentration of about 20 mM.

In one embodiment the buffer substance in the first buffered solution and the buffer substance in the second buffered solution are the same buffer substance. In one embodiment the buffer substance in the first solution and the buffer substance in the second solution are different buffer substances. In one embodiment the first solution has a pH value of about pH 3.5 to about pH 7.5. In one embodiment the first solution has a pH value of about pH 5 to about pH 6. In one embodiment the first solution has a pH value of about pH 5.5.

In one embodiment the second solution has a pH value of about pH 7.0 to about pH 9.5. In one embodiment the second solution has a pH value of about pH 8 to about pH 9. In one embodiment the second solution has a pH value of about pH 8.2 to about pH 8.8.

An exemplary first solution comprises 20 mM MES and 150 mM NaCl, adjusted to pH 5.5. An exemplary second solution comprises 20 mM TRIS and 150 mM NaCl, adjusted to pH 8.8 An exemplary second solution comprises 20 mM HEPES adjusted to pH 8.6. An exemplary second solution comprises 20 mM TRIS adjusted to pH 8.2.

In one embodiment the buffered solution comprises an additional salt. In one embodiment the additional salt is selected from sodium chloride, sodium sulphate, potassium chloride, potassium sulfate, sodium citrate, or potassium citrate. In one embodiment the buffered solution comprises from 50 mM to 1000 mM of the additional salt. In one embodiment the buffered solution comprises from 50 mM to 750 mM of the additional salt. In one embodiment the buffered solution comprises from 50 mM to 500 mM of the additional salt. In one embodiment the buffered solution comprises from 50 mM to 750 mM of the additional salt. In one embodiment the buffered solution comprises about 50 mM to about 300 mM of the additional salt.

In one embodiment the first and/or second solution comprises sodium chloride. In one embodiment the first and/or second solution comprises of about 50 mM to about 300 mM sodium chloride.

### 5.5.3 Washing

In some embodiments, substantial nanovesicle purity can be achieved by flowing through impurities during the column loading phase, eluting impurities during selective excipient washes and selectively eluting product during elution while leaving additional impurities bound to the column. Absorbance measured from column eluates can indicate purify of nanovesicle obtained by the methods.

Optionally, purity of nanovesicles comprising FcRn binding polypeptides can be further improved by washing samples prior to elution. In some embodiments, excipient can be a washing buffer. The excipient can be a solution having specific pH ranges, salts, organic solvents, small molecules, detergents, zwitterions, amino acids, polymers, and any combination of the above.

More specifically, the excipient can comprise arginine, lysine, glycine, histidine, calcium, sodium, lithium, potassium, iodide, magnesium, iron, zinc, manganese, urea, propylene glycol, aluminum, ammonium, guanidinium polyethylene glycol, EDTA, EGTA, a detergent, chloride, sulfate, carboxylic acids, sialic acids, phosphate, acetate, glycine, borate, formate, perchlorate, bromine, nitrate, dithiothreitol, beta mercaptoethanol, or tri-n-butyl phosphate.

The excipient can also comprise a detergent, selected from the group consisting of cetyl trimethylammonium chloride, octoxynol-9, TRITON™ X-100 (i.e., polyethylene glycol p-(1, 1,3,3-tetramethylbutyl)-phenyl ether) and TRITON™ CG-110 available from Sigma-Aldrich; sodium dodecyl sulfate; sodium lauryl sulfate; deoxycholic acid; Polysorbate 80 (i.e., Poly oxy ethylene (20) sorbitan monooleate); Polysorbate 20 (i.e., Poly oxy ethylene (20) sorbitan monolaurate); alcohol ethoxylate; alkyl polyethylene glycol ether; decyl glucoside; octoglucosides; SafeCare; ECOSURF™ EH9, ECOSURF™ EH6, ECOSURF™ EH3, ECOSURF™ SA7, and ECOSURF™ SA9 available from DOW Chemical; LUTENSOL™ M5, LUTENSOL™ XL, LUTENSOL™ XP and APG™ 325N available from BASF; TOMADOL™ 900 available from AIR PRODUCTS; NATSURF™ 265 available from CRODA; SAFECARE™1000 available from Bestchem, TERGITOL™ L64 available from DOW; caprylic acid; CHEMBETAINE™ LEC available from Lubrizol; and Mackol DG.

In some embodiments, further methods to improve the purification outcome can be applied. For example, the amount of nanovesicles comprising FcRn binding polypeptides that can be loaded per volume of chromatographic resin can be improved by modulating the feed material, for example, by increasing the concentration of FcRn binding nanovesicles, decreasing the concentration of impurities, altering the pH, decreasing the salt concentrations, decreasing the ionic strength, or altering the specific sub-populations of nanovesicles comprising FcRn binding polypeptides. In certain embodiments, owing to mass transfer constraints and slow adsorption and desorption of nanovesicles on the resin, the amount of nanovesicles comprising FcRn binding polypeptides that can be loaded per volume of chromatographic resin can be increased by slowing the flow rate during column loading, employing longer columns to increase the residence time.

In other embodiments, an isolated nanovesicle comprising FcRn binding polypeptide composition has an amount and/or concentration of desired FcRn binding nanovesicles (e.g. EVs) at or above an acceptable amount and/or concentration. In other embodiments, the isolated nanovesicle comprising FcRn binding polypeptide composition is enriched as compared to the starting material (e.g., producer cell conditioned media) from which the composition is obtained. This enrichment can be of 10%, 20%, 30%, 40%, 50%, 60%, 70%, 80%, 90%, 95%, 96%, 97%, 98%, 99%, 99.9%, 99.99%, or greater than 99.9999% compared to the starting material.

In some embodiments, isolated nanovesicles comprising FcRn binding polypeptides preparations are substantially free of residual biological products. In some embodiments, the isolated nanovesicle comprising FcRn binding polypeptide preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the nanovesicles comprising FcRn binding polypeptide contains no detectable producer cells and that only FcRn binding nanovesicles are detectable.

In a further aspect, a chromatography method comprising FcRn as a second binding partner as described herein can be used for the isolation/enrichment of crude mixtures of hybridosomes comprising FcRn binding polypeptides from unfused lipid nanoparticles. In some embodiments, isolated hybridosomes comprising FcRn binding polypeptides preparations are substantially free of unfused lipid nanoparticles. In some embodiments, the isolated hybridosomes comprising FcRn binding polypeptide preparations are 100% free, 99% free, 98% free, 97% free, 96% free, 95% free, 94% free, 93% free, 92% free, 91% free, or 90% free of any unfused lipid nanoparticles.

### 5.6 Compositions and Kits

In another aspect, compositions and kits are provided, comprising a polypeptide, nanovesicle, nucleic acid, expression vector, and/or a cell of the disclosure (e.g., as described in Sections 5.2-5.4). Such compositions can, e.g., be a cosmetic, a diagnostic, or a pharmaceutical composition.

In some embodiments, a pharmaceutical composition comprises a FcRn binding polypeptide as described herein (e.g, a FcRn binding polypeptide comprising a transmembrane protein and a modified Fc domain that can specifically bind FcRn and does not form homodimers) and further comprises one or more pharmaceutically acceptable carriers and/or excipients. Guidance for preparing formulations can be found in any number of handbooks for pharmaceutical preparation and formulation that are known to those of skill in the art.

In certain embodiments, a composition as described herein is useful as a medicament. Typically, such a medicament includes a therapeutically effective amount of a composition provided herein. Accordingly, a respective composition can be used for the production of a medicament useful in the treatment of disorders. Thus, in one embodiment, pharmaceutical compositions and kits comprising a polypeptide, nanovesicle, nucleic acid, expression vector, and/or a cell of the disclosure are provided. In some embodiments, provided are pharmaceutical compositions and kits comprising a nanovesicle of the disclosure (i.e., a nanovesicle comprising a polypeptide as described above).

In some embodiments, a pharmaceutical composition comprises a polypeptide, nanovesicle, nucleic acid, expression vector, and/or a cell described herein and further comprises one or more pharmaceutically acceptable carriers, excipients and/or diluent. Guidance for preparing formulations can be found in any number of handbooks for pharmaceutical preparation and formulation that are known to those of skill in the art.

Pharmaceutically acceptable carriers include any solvents, dispersion media, or coatings that are physiologically compatible and that preferably do not interfere with or otherwise inhibit the activity of the active agent. Various pharmaceutically acceptable excipients are well-known in the art.

In some embodiments, the pharmaceutically acceptable carrier is suitable for intravenous, intramuscular, oral, intraperitoneal, intrathecal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acids or glutathione, chelating agents, low molecular weight proteins, compositions that reduce the clearance or hydrolysis of the active agents, or excipients or other stabilizers and/or buffers. Other pharmaceutically acceptable carriers and their formulations are well-known in the art.

Pharmaceutical compositions can be manufactured in a manner that is known to those of skill in the art, e.g., by means of conventional mixing, dissolving, granulating, dragee-making, emulsifying, encapsulating, entrapping or lyophilizing processes. The methods and excipients disclosed herein are merely exemplary and are in no way limiting.

For oral administration, a FcRn binding polypeptide as disclosed herein can be formulated by combining it with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

A FcRn binding polypeptide as disclosed herein can be formulated for parenteral administration by injection, e.g., by bolus injection or continuous infusion. For injection, the FcRn binding polypeptide can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. In some embodiments, FcRn binding polypeptides can be formulated in aqueous solutions, preferably in physiologically compatible buffers such as Hanks's solution, Ringer's solution, or physiological saline buffer. Formulations for injection can be presented in unit dosage form, e.g, in ampules or in multi-dose containers, with an added preservative. The compositions can take such forms as suspensions, solutions or emulsions in oily or aqueous vehicles, and can contain formulatory agents such as suspending, stabilizing and/or dispersing agents.

In some embodiments, a FcRn binding polypeptide as disclosed herein is prepared for delivery in a sustained-release, controlled release, extended-release, timed-release or delayed-release formulation, for example, in semi-permeable matrices of solid hydrophobic polymers containing the active agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients. Sustained-release delivery systems can, depending on their design, release the compounds over the course of hours or days, for instance, over 4, 6, 8, 10, 12, 16, 20, or 24 hours or more. Usually, sustained release formulations can be prepared using naturally occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

Typically, a pharmaceutical composition for use in in vivo administration is sterile. Sterilization can be accomplished according to methods known in the art, e.g., heat sterilization, steam sterilization, sterile filtration, or irradiation.

Dosages and desired drug concentration of pharmaceutical compositions of the disclosure may vary depending on the particular use envisioned. The determination of the appropriate dosage or route of administration is well within the skill of one in the art. Exemplary suitable dosages are also described in Section 5.7 below.

### 5.7 Therapeutic and Diagnostic Uses

The nanovesicles comprising the polypeptide of the present disclosure (e.g., as described in Section 5.4), as well as nucleic acids and expression vectors encoding such polypeptides (e.g., as described in Section 5.3), cells capable of expressing such polypeptides (e.g., as described in Section 5.3), and compositions and kits comprising the foregoing (e.g., as described in Section 5.6) may be used for treating, monitoring, preventing and/or diagnosing a number of diseases and disorders (e.g. cancer, inflammation, or inflammation associated with cancer).

Thus, in one aspect, provided herein is a method of delivering a therapeutic or diagnostic agent to a target cell or tissue, wherein the method comprises providing an extracellular vesicle or hybridosome described herein to said target cell or tissue.

In one aspect, a method of treating a disease or disorder is provided. The method comprises the steps of administering a pharmaceutically effective amount of a composition as described herein (i.e. a composition comprising or capable of expressing a polypeptide) to a subject in need thereof. In one embodiment, the method comprises administering a pharmaceutically effective amount of a pharmaceutical composition described above.

The subject in need of a treatment can be a human or a non-human animal. Typically, the subject is a mammal, e.g., an ape, a dog, a guinea pig, a horse, a monkey, a mouse, a pig, a rabbit or a rat. In case of an animal model, the animal might be genetically engineered to develop a disorder or to show the characteristics of a disease.

In some embodiments, the subject has a cancer, an inflammatory disorder, autoimmune disease, a chronic disease, inflammation, damaged organ function, an infectious disease, metabolic disease, degenerative disorder, genetic disease (e.g., a genetic deficiency, a recessive genetic disorder, or a dominant genetic disorder), or an injury. In some embodiments, the subject has an infectious disease and the nanovesicle comprises an antigen for the infectious disease. In some embodiments, the subject has a genetic deficiency and the nanovesicle comprises a protein for which the subject is deficient, or a nucleic acid (e.g., mRNA) encoding the protein, or a DNA encoding the protein, or a chromosome encoding the protein, or a nucleus comprising a nucleic acid encoding the protein. In some embodiments, the subject has a dominant genetic disorder, and the nanovesicle comprises a nucleic acid inhibitor (e.g., shRNA, siRNA or miRNA) of the dominant mutant allele. In some embodiments, the subject has a dominant genetic disorder, and/or the nanovesicle comprises a nucleic acid inhibitor (e.g., shRNA, siRNA or miRNA) of the dominant mutant allele, and/or the nanovesicle also comprises an mRNA encoding a non-mutated allele of the mutated gene that is not targeted by the nucleic acid inhibitor. In some embodiments, the subject is in need of vaccination. In some embodiments, the subject is in need of regeneration, e.g., of an injured site.

In some embodiments, the nanovesicle or composition described herein is administered to the subject at least 1, 2, 3, 4, or 5 times.

In some embodiments, the nanovesicle comprising a polypeptide described herein targets a tissue, e.g., liver, lungs, heart, spleen, pancreas, gastrointestinal tract, kidney, testes, ovaries, brain, reproductive organs, central nervous system, peripheral nervous system, skeletal muscle, endothelium, inner ear, or eye, when administered to a subject, e.g., a mouse or human. In some embodiments, at least 0.1%, 0.5%, 1%, 1.5%, 2%, 2.5%, 3%, 4%, 5%, 10%, 20%, 30%, 40%, 50%, 60%, 70%, 80%, or 90% of the nanovesicles comprising a polypeptide described herein in an administered composition are present in the target tissue after 24, 48, or 72 hours.

In some embodiments, the nanovesicle or composition as described above is administered to a subject at a therapeutically effective amount or dose. Illustrative dosages include a daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient. In some embodiments, a patient initially is given a low dose, which is then increased to an efficacious dosage tolerable to the patient. Determination of an effective amount is well within the capability of those skilled in the art.

In some embodiments, the nanovesicles or compositions as disclosed herein are used for the treatment of cancer. In certain embodiments, the cancer is a primary cancer of the CNS, such as glioma, glioblastoma multiforme, meningioma, astrocytoma, acoustic neuroma, chondroma, oligodendroglioma, medulloblastomas, ganglioglioma, Schwannoma, neurofibroma, neuroblastoma, or an extradural, intramedullary or intradural tumor. In some embodiments, the cancer is a solid tumor, or in other embodiments, the cancer is a non-solid tumor. Solid-tumor cancers include tumors of the central nervous system, breast cancer, prostate cancer, skin cancer (including basal cell carcinoma, cell carcinoma, squamous cell carcinoma and melanoma), cervical cancer, uterine cancer, lung cancer, ovarian cancer, testicular cancer, thyroid cancer, astrocytoma, glioma, pancreatic cancer, mesotheliomas, gastric cancer, liver cancer, colon cancer, rectal cancer, renal cancer including nephroblastoma, bladder cancer, oesophageal cancer, cancer of the larynx, cancer of the parotid, cancer of the biliary tract, endometrial cancer, adenocarcinomas, small cell carcinomas, neuroblastomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcome soft tissue sarcomas, osteosarcomas, peripheral primitive neuroectodermal tumors, retinoblastomas, and rhabdomyosarcomas. In some embodiments, the use of a nanovesicle as disclosed herein in the manufacture of a medicament for treating cancer is provided.

In some embodiments, the nanovesicles or compositions as disclosed herein may be used in the treatment of an autoimmune or inflammatory disease. Examples of such diseases include, but are not limited to, ankylosing spondylitis, arthritis, osteoarthritis, rheumatoid arthritis, psoriatic arthritis, asthma, scleroderma, stroke, atherosclerosis, Crohn's disease, colitis, ulcerative colitis, dermatitis, diverticulitis, fibrosis, idiopathic pulmonary fibrosis, fibromyalgia, hepatitis, irritable bowel syndrome (IBS), lupus, systemic lupus erythematous (SLE), nephritis, multiple sclerosis, and ulcerative colitis. In some embodiments, the use of a nanovesicle as disclosed herein in the manufacture of a medicament for treating an autoimmune or inflammatory disease is provided.

In some embodiments, the nanovesicles or compositions as disclosed herein may be used in the treatment of a cardiovascular disease, such as coronary artery disease, heart attack, abnormal heart rhythms or arrhythmias, heart failure, heart valve disease, congenital heart disease, heart muscle disease, cardiomyopathy, pericardial disease, aorta disease, marfan syndrome, vascular disease, or blood vessel disease.

The nanovesicles or compositions of the present disclosure may be administered to a human or animal subject via various different administration routes, for instance auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracerebroventricular, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the nanovesicle, composition, and/or the therapeutic molecule.

A nanovesicle as disclosed herein may be used for detection or diagnostic purposes in vivo and/or in vitro which encompasses quantitative and/or qualitative detection. Likewise, a polypeptide, a nucleic acid, an expression vector and/or a cell described in the preceding text can be used accordingly as detailed in this section.

For diagnostic applications or detection purposes, the nanovesicle may include a moiety that is detectable, e.g., detectable through biological imaging, including radiology or magnetic resonance imaging. In some embodiments, the nanovesicle comprises a reporter protein or a detectable label. In some embodiments, the nanovesicle as disclosed herein is coupled to one or more substances that can be recognized by a detector substance. By way of example, the nanovesicle may be covalently linked to biotin, which can be detected by means of its capability to bind to streptavidin.

In certain embodiments, the nanovesicle is useful for detecting its presence in a sample, preferably a sample of biological origin, such as, e.g., from a human subject. Non-limiting examples of biological samples include blood, biopsy, cerebrospinal fluid, lymph, urine, and/or non-blood tissues. In certain embodiments, a biological sample includes a cell or tissue from human patients.

Thus, in some aspects, methods are provided, including the steps of: (i) contacting a subject or a biological sample with a nanovesicle of the disclosure comprising a detectable moiety; (ii) allowing for the nanovesicle to interact with the subject or sample; and (iii) detecting the nanovesicle. Such methods may be in vitro or in vivo methods. In some embodiments, such methods are methods for localizing a nanovesicle.

### 6. EXAMPLES

The examples illustrate the methods and compositions disclosed herein. It is understood that various other embodiments may be practiced, given the general description provided above.

### 6.1 Example 1: Production of Engineered EVs

The DNA sequence for fusion proteins comprising a FcRn bind site fused to a transmembrane scaffold protein (EphA4) were designed in silico. The DNA sequences encoded for polypeptides with the following architecture:

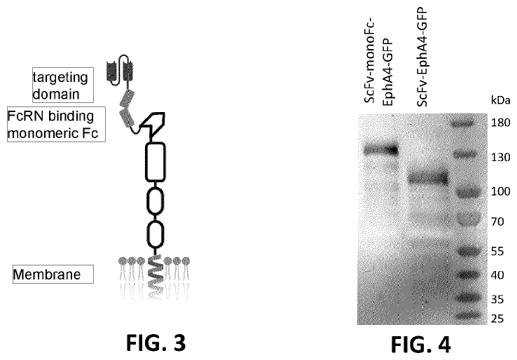

Fusion protein 1: EphA4 signal peptide-scFv-linker1-modified monomeric Fc-linker2-EphA4 fragment-linker3-EGFP, with an extracellular domain as depicted in FIG. 3, and

Fusion protein 2: EphA4 signal peptide-scFv-linker1-EphA4 fragment-linker3-EGFP.

The DNA sequences were synthesized and cloned into a lentiviral backbone comprising an internal ribosome entry site and antibiotic selection marker, by a commercial DNA synthesis vendor. Lenti particles were produced using a standard protocol and HEK293T cells were transduced and then sorted by flow cytometry for GFP expression and then monoclonally expanded.

The cell lines were cultivated and EVs were isolated from the supernatant of cultures of stable clones. Specifically, EV-containing media was collected and clarified from debris by differential centrifugation. The supernatant was then filtered with a 0.22 um syringe or bottle-top filter and further processed by different purification steps. For larger scale productions, high density cultures were maintained in a stirred bioreactor in perfusion mode, whereby the harvested perfusion supernatant was pre-clarified and filtered by an alternating tangential flow system fitted with a 0.2 um hollow fiber filter. EVs were isolated and purified from the clarified conditioned media using a variety of methods, typically a combination of dia-/ultrafiltration with tangential flow filtration (TFF) and flow through based multimodal chromatography and/or bind and elute chromatography steps. Purified EVs were then frozen and stored for downstream analysis. Western blotting was carried out on a purified EVs. As shown in FIG. 4, equal protein amounts of fusion protein 1 (left lane) and fusion protein 2 (right 2) expressed on EVs were loaded on a denaturing polyacrylamide gel. Western blotting for EphA4, using antibodies specific for EphA4 extracellular domain, demonstrated that fusion proteins 1 and 2 were expressed on the engineered EVs.

### 6.2 Example 2: Selection of FcRn Binding Polypeptide Expressing Source Cells

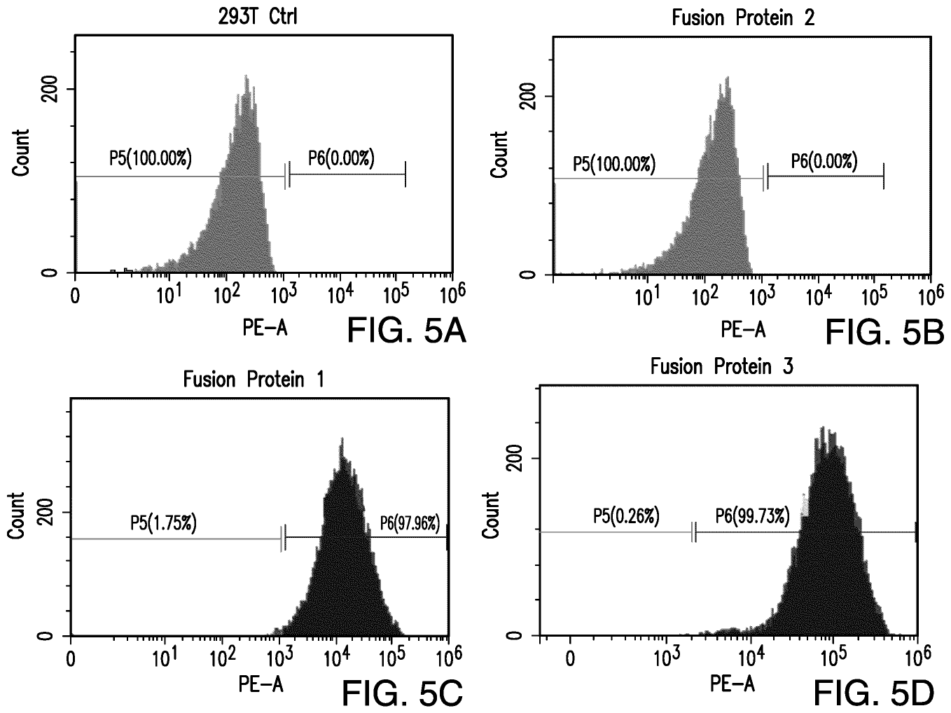

In addition to the producer cell lines generated in Example 1, a stable pool of cells expressing a FcRn polypeptide (fusion protein 3) comprising from N- to C-Terminus: a targeting monobody-linker1-modified monomeric Fc-linker2-EphB2 scaffold-linker3-turboluc (wherein the EphB2 scaffold comprised residues 195-905 of EphB2, lacking a LBD, and containing the following amino acid substitutions L356A I395A S536E A562S, Y822F relative to wild type EphB2), was generated using the same lenti backbone. See SEQ ID NO: 73 for the sequence of the full fusion protein. In contrast to the previous two GFP tagged cell lines, the FcRn binding polypeptide expressed by this cell line does not contain a GFP tag that would enable flow cytometry assisted cell sorting (FACS). In order to select a high expressing cell clone, the transduced cells were monoclonally expanded using a limit dilution method and antibiotic selection. Different clones were screened for FcRn binding polypeptide expression level by flow cytometry using a fluorescent anti-human Fc domain antibody (Invitrogen catalog 12-4998-82). As shown in FIGS. 5A-5D, HEK293T control cells and the cell line expressing fusion protein 2 (as described in example 1) (FIG. 5A and FIG. 5B, respectively) were not stained by the anti-human Fc domain antibody while all cells of the cell line expressing fusion protein 1 (as described in example 1, comprising a modified monomeric Fc) and all cells of a selected clone expressing the FcRn binding polypeptide of this example were successfully stained by a fluorescent anti-human Fc domain antibody (FIG. 5C and FIG. 5D, respectively).

### 6.3 Example 3: Low pH Elution of Engineered EVs

The high expressing clone of producer cell generated in Example 2 was cultivated in serum free, chemically defined media and engineered EVs were isolated from the supernatant as described in Example 1. Isolated engineered EVs were loaded onto a protein A affinity chromatography column. Flow rate settings for column equilibration, sample loading and column cleaning in place procedure were chosen according to the manufacturer's instructions. An elution buffer comprising 0.1 M glycine-HCl, pH 3.0 was utilized to elute the protein A bound FcRn binding EVs into a pre-plated pH neutralization buffer. The flow through and elution were fractionated into a 96 well plate and sampled for particle counts by dynamic light scattering (DLS). Compared to the amount of particles loaded onto the column, both UV absorbance and particle/ml measurement in both the flow through and elution fractions were minimal, indicating a low yield of recovery.

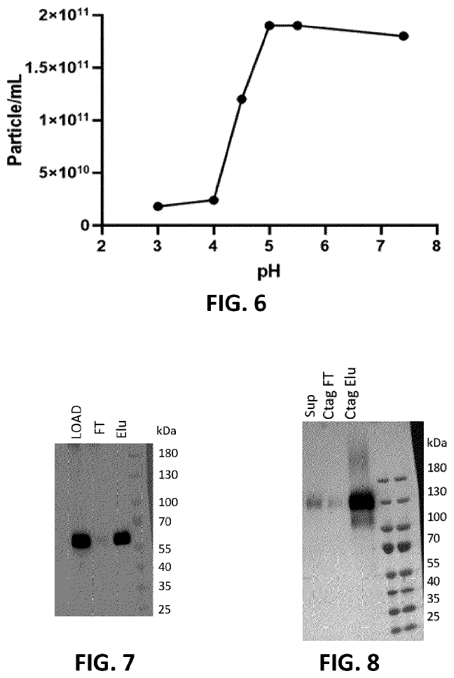

To investigate the low recovery rate, purified EV samples of approximately 1.8×10¹² particles/ml were diluted 1:10 into buffers with decreasing pH, mixed thoroughly and incubated for 20 minutes at room temperature. Incubated EV samples were then diluted into a buffer with neutral pH (50 mM Tris, 50 mM NaCl) at a dilution factor to achieve a particle concentration in the linear range on the nanoparticle tracking analysis (NTA) instrument (between 1:2500 at higher pHs to 1:500 at low pHs, respectively) and samples were then measured for particle concentration. As shown in FIG. 6, mere incubation of engineered EVs at a pH below 5, which subsequently returned to pH 7.4, resulted in an approximately 90% decrease in particle concentration (from approximately 1.8×10¹¹ to approximately 1.8×10¹⁰) and large aggregates were visible in the measurement video. Aggregation was not reversed by diluting the EVs into high pH buffer. Accordingly, EV elution below pH 5 may lead to irreversible aggregation, making affinity purification methods requiring very low pH suboptimal.

### 6.4 Example 4: Constructions of Single Chain FcRn Expression Vector

Recombinant human and mouse single chain FcRn (scFcRn) constructs containing the mouse IgG kappa chain leader sequence as the secretion signal followed by the mature B2M sequence connected through 3x(GGGGS) (SEQ ID NO: 72) to the mature sequence of the FCGRT heavy chain fused to a C-tag were designed in silico, synthesized by a commercial DNA synthesis vendor and cloned into a lenti and transient vector.

### 6.5 Example 5: Production of Recombinant scFcRn

Recombinant human scFcRn and mouse scFcRn polypeptides were respectively expressed in HEK293 cells grown in customized chemically defined culture media (containing only small molecules). Expression of scFcRn in concentrated supernatant was detected by western blot (Human: Invitrogen PA5-97738 antibody, Mouse R&D Systems AF6775). For large scale production, a stable cell line expressing recombinant scFcRn was generated using the vector of Example 4. Said cell line was cultured in a hollow fiber cartridge (5 kDa cutoff) or an orbital shaker. The clarified supernatant or partially purified clarified supernatant (concentrated, and diafiltered against PBS on a tangential flow device with 10 kDa hollow fiber unit) was loaded onto Capture-Select C-TagXL affinity chromatography column. The column was washed with PBS and scFcRn was eluted either by i) 20 mM Tris, 2.0M MgCl₂ pH 7.4, ii) 50 mM acetic acid pH 3.0, or iii) 20 mM Tris, 2 mM “S-E-P-E-A” peptide, pH 7.4. For scFcRn eluted by “S-E-P-E-A” peptide, elution fractions were dialyzed or desalted to remove the peptide. Protein content of purified scFcRn was measured by bicinchoninic acid assay (BCA) and product was stored at −20° C. The purity of scFcRn was examined by western blot and SDS-Page. As shown in FIG. 7, western blotting of the clarified supernatant (lane 1), the flow through fractions (lane 2) and the elution fractions (lane 3), using a mouse FcRn specific antibody, confirmed binding and elution of the scFcRn product. Alternatively, to increase purity and selection of functional protein, a two-step affinity chromatography was performed. In a first step, the crude clarified supernatant or partially purified clarified supernatant, as above, was adjusted to pH 5.8 with HCl, filtered through 0.45 μm filter and loaded onto commercially available hIgG-sepharose column that was previously equilibrated with MES buffer pH 5.8. The column was washed with 5 column volumes of MES buffer pH 5.8. Finally, the bound protein was eluted from the column with pH 8.0 buffer (50 mM Tris, pH 8.0, 100 mM NaCl). Purified protein was then loaded onto a Capture-Select C-TagXL column, washed with PBS and optionally eluted with i) 20 mM Tris, 2.0M MgCl₂ pH 7.4, ii) 50 mM acetic acid pH 3.0, or iii) 20 mM Tris, 2 mM “S-E-P-E-A” peptide, pH 7.4 as above.

### 6.6 Example 6: scFcRn Purification of Engineered EVs

Recombinant scFcRn protein of Example 5 was loaded to a C-tagXL column following procedures from the instruction manual. The resin was then washed with 25 mM MES pH 5.8, 150 mM NaCl. A stable cell line, expressing a polypeptide comprising from N- to C-Terminus: a targeting monobody-linker-modified monomeric Fc-linker-EphA4 fragment (containing residues 29-590 of EphA4 and an amino acid substitution of F154A relative to wild-type EphA4, was generated and cultivated, and the supernatant was collected, clarified and concentrated. The pH of the harvested supernatant was adjusted to pH 5.8 and then loaded on the equilibrated column and further washed with 25 mM MES pH 5.8, 150 mM NaCl. Bound sample was eluted with 50 mM Tris pH 7.4, 150 mM NaCl (reverse flow).

To confirm the presence of the transmembrane FcRn binding polypeptide in the eluted flow through and elution sample, the elute fractions were pooled and concentrated and subsequently were probed by western blot using an anti-EphA4 antibody (ECM Biosciences, Cat. No. EM2801). As shown in FIG. 8, the elution sample showed an enrichment in EphA4 signal.

### 6.7 Example 7: pH Dependent Enrichment by scFcRn

Approximately 5 mg of scFcRN protein per 1 ml resin material was covalently coupled to POROS 20 EP resin following procedures from the instruction manual. The resin was then washed with 10 column volumes of 0.2M Tris at pH 8.2 with 500 mM NaCl, followed by 10 column volumes of 25 mM Tris pH 8.2 as instructed by the manufacture's manual. After final wash with Tris-NaCl followed by equilibrating the column with MES buffer pH 5.8, it was ready to use. Successful resin functionalization was confirmed by loading purified human IgG1.

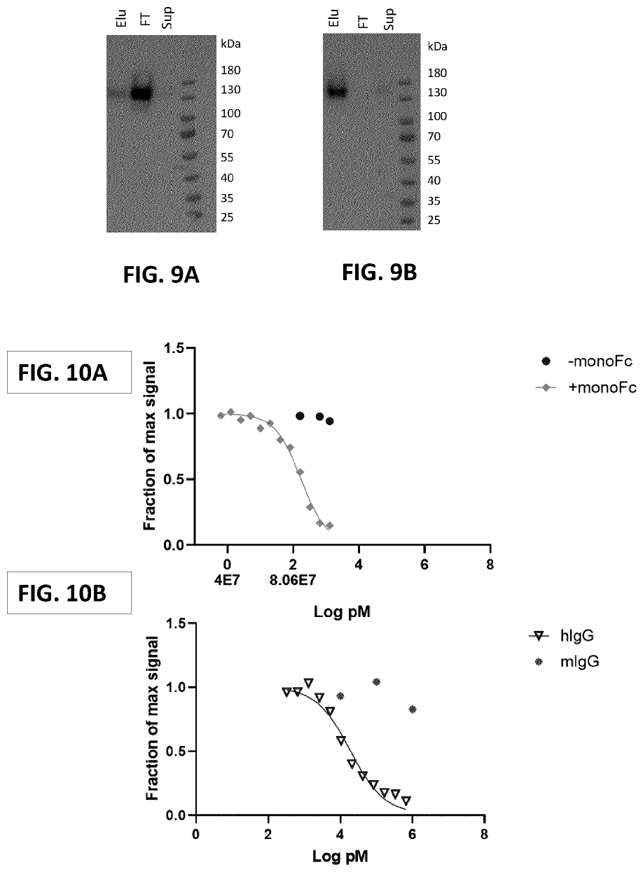

The pH dependency of enrichment of engineered EVs comprising an FcRn binding polypeptide was determined by loading onto the scFcRn functionalized POROS EP resin preequilibrated with 50 mM Tris pH 7.4, 150 mM NaCl, the clarified and diafiltrated supernatant of the producer cell expressing a FcRn binding polypeptide comprising the transmembrane EphA4 scaffold protein (i.e., fusion protein 1 as described in Example 1) without prior adjustment of the pH. Alternatively, a sample of the same batch of supernatant was pH adjusted as described in Example 6 prior to loading onto the scFcRn functionalized POROS EP resin preequilibrated with 25 mM MES pH 5.8. The columns were washed and eluted as described in Example 6. In both cases, the flow through and elution fractions were pooled and concentrated. To confirm the presence of the transmembrane FcRn binding polypeptide in the eluted flow through and elution sample, the flow through elute fractions were pooled and concentrated and were subsequently probed by western blot using an anti-EphA4 antibody (ECM Biosciences, Cat. No. EM2801). The clarified supernatant was not concentrated. As shown in FIG. 9A, for the non-pH adjusted supernatant the pooled and concentrated flow through fractions retained the transmembrane EphA4 while for the acidified supernatant, in FIG. 9B the pooled and concentrated elution fractions showed an enrichment of FcRn binding polypeptide comprising the transmembrane EphA4 scaffold protein.

### 6.8 Example 8: FcRn Binding Immunoassay

A Lumit™ FcRn Binding Immunoassay (Promega) was performed with purified EVs described in Example 2 and native Hek293 EVs. Samples of said EVs and a human IgG1 and a mouse IgG1 as controls were each serially diluted and incubated with a split FcRn/Tracer according to the manufacturer instructions (Tracer and FcRn were 10× diluted). Detection reagent was added and luminescence was detected on a plate reader. As shown in FIG. 10A, purified EVs described in Example 1 were able to bind FcRn while native EVs did not bind to FcRn. As shown in FIG. 10B, human IgG1 was able to bind FcRn while mouse IgG1 did not.

### 6.9 Example 9: Blood Clearance after IV Administration of Modified Fc Hybridosomes

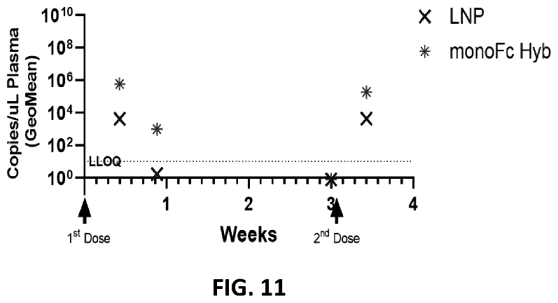

EVs (e.g. exosomes) are considered to have a very short half-life and circulation time. To test the blood clearance of hybridosomes comprising EphB2 scaffold described in Example 2, nude immunocompetent SKH1 mice (6-8 weeks old, n=6/group) were injected intravenously with DNA loaded lipid nanoparticles or hybridosomes (0.5 mg/kg). The DNA cargo encoded a promoter, a reporter transgene and a BGH poly(A). The lipid nanoparticles were prepared on a Nanoassemblr™ microfluidic system (Precision NanoSystems) according to the manufacturer's instructions. Animals were re-dosed on day 21, post administration. In order to monitor blood clearance, on days 3, 6, 21 (pre-second dose) and 24, respectively, twenty microliters of blood were drawn from the tail vein and processed to plasma. Two microliters of diluted plasma were used in a Taqman qPCR assay to quantify the DNA sequence, specifically the BGH Poly A sequence, by comparing against a standard curve on the same plate. Recovery efficiency of DNA from naïve mouse plasma was determined by spiking the DNA vector into mouse plasma. As shown in FIG. 11, hybridosomes comprising a targeting monobody-modified Fc domain fused to a EphB2 scaffold protein could be detected in the mouse plasma 6 days post administration while on the same day the plasma copy number was below the detection limit for the LNP treated group.

| SEQ | |
|---|---|
| ID | |
| NO | Additional Sequences |
| 73 | MALRRLGAALLLLPLLAAVSDVPRDLEVVAATPTSLLISWYYPFCA |
| | FYYRITYGETGGNSPVQEFTVPRSPDTATISGLKPGVDYTITVYAV |
| | TCLGSYSRPISINYRTGGGGSGGGGSGGAPEAAGGPSVFLFPPKPK |
| | DTLMISRTPEVTCVVVDVSHEDPEVKFNWYVDGVEVHNAKTKPREE |
| | QYNSTYRVVSVLTVLHQDWLNGKEYKCKVSNKALGAPIEKTISKAK |
| | GQPREPQVYTKPPSRDELTKNQVSLSCLVKGFYPSDIAVEWESNGQ |
| | PENNYKTTVPVLDSDGSFRLASYLTVDKSRWQQGNVFSCSVMHEAL |
| | HNHYTQKSLSLSPGGGSGGGSGGGSGGGSRKCPRIIQNGAIFQETL |
| | SGAESTSLVAARGSCIANAEEVDVPIKLYCNGDGEWLVPIGRCMCK |
| | AGFEAVENGTVCRGCPSGTFKANQGDEACTHCPINSRTTSEGATNC |
| | VCRNGYYRADLDPLDMPCTTIPSAPQAVISSVNETSLMLEWTPPRD |
| | SGGREDAVYNIICKSCGSGRGACTRCGDNVQYAPRQLGLTEPRIYA |
| | SDLLAHTQYTFEIQAVNGVTDQSPFSPQFASVNITTNQAAPSAVSI |
| | MHQVSRTVDSITLSWSQPDQPNGVILDYELQYYEKELSEYNATAIK |
| | SPTNTVTVQGLKAGAIYVFQVRARTVAGYGRYSGKMYFQTMTEAEY |
| | QTEIQEKLPLIIGSSAAGLVFLIAVVVISIVCNRRGFERADSEYTD |
| | KLQHYTSGHMTPGMKIYIDPFTYEDPNEAVREFAKEIDISCVKIEQ |
| | VIGAGEFGEVCSGHLKLPGKREIFVAIKTLKSGYTEKQRRDFLSEA |
| | SIMGQFDHPNVIHLEGVVTKSTPVMIITEFMENGSLDSFLRQNDGQ |
| | FTVIQLVGMLRGIAAGMKYLADMNYVHRDLAARNILVNSNLVCKVS |
| | DFGLSRFLEDDTSDPTYTSALGGKIPIRWTAPEAIQYRKFTSASDV |
| | WSYGIVMWEVMSFGERPYWDMTNQDVINAIEQDYRLPPPMDCPSAL |
| | HQLMLDCWQKDRNHRPKFGQIVNTLDKMIRNPNSLKAMAPLSSGIN |
| | LPLGGGEAEAERGKLPGKKLPLEVLIELEANARKAGCTRGCLICLS |
| | KIKCTAKMKKYIPGRCADYGGDKKTGQAGIVGAIVDIPEISGFKEM |
| | EPMEQFIAQVDRCADCTTGCLKGLANVKCSDLLKKWLPGRCATFAD |
| | KIQSEVDNIKGLAGD |

What is claimed is:

1. A polypeptide, wherein the polypeptide comprises:
a. a transmembrane domain; and
b. a modified Fe domain of an immunoglobulin that
i. is capable of specifically binding to the Fc binding site of an FcRn; and
ii. lacks the ability to form homodimers.

2. The polypeptide of claim 1, wherein the equilibrium dissociation constant of the modified Fc domain bound to FcRn at a pH of 6.5 has a value of at most 10⁻⁴M.

3. The polypeptide of claim 1 or 2, wherein the equilibrium dissociation constant of the modified Fc domain bound to FcRn at a pH of 7.4 has a value of at least 10⁻⁴M.

4. The polypeptide of any one of claims 1-3, wherein the modified Fc domain is capable of specifically binding to the amino acid sequence between position 135-158 of human FcRn (SEQ ID NO: 7) and/or mouse FcRn (SEQ ID NO: 8).

5. The polypeptide of any one of claims 1-4, wherein the modified Fc domain is capable of specifically binding to the amino acid sequence LNGEEFMX₁FX₂X₃X₄X₅GX₆WX₇GX₈W (SEQ ID NO:6), wherein X₁, X₂, X₃, X₄, X₅, X₆, X₇ and X₈ each is any amino acid.

6. The polypeptide of any one of claims 1-5, wherein said polypeptide does not substantially bind to C1q, FcγRI, FcγRII or FcγRIII.

7. The polypeptide of any one of claims 1-6, wherein:
a. the complement dependent cytotoxicity (CDC) activity of the modified Fc domain;
b. the antibody dependent cell mediated cytotoxicity (ADCC) activity of the modified Fc domain;
c. the antibody dependent cell mediated phagocytosis (ADCP) activity of the modified Fc domain; and/or
d. the antibody dependent intracellular neutralization (ADIN) activity of the modified Fc domain
is decreased by at least 10%, 20%, 30%, 40%, or 50% compared to the unmodified Fc domain.

8. The polypeptide of any one of claims 1-7, wherein:
a. the complement dependent cytotoxicity (CDC) activity of the modified Fc domain;
b. the antibody dependent cell mediated cytotoxicity (ADCC) activity of the modified Fc domain;
c. the antibody dependent cell mediated phagocytosis (ADCP) activity of the modified Fc domain; and/or
d. the antibody dependent intracellular neutralization (ADIN) activity of the modified Fc domain
is decreased by at least 1.5, 2, 3, 4, or 5-fold, compared to the unmodified Fc domain.

9. The polypeptide of any one of claims 1-8, wherein the FcRn binding polypeptide comprises from N-terminus to C-terminus:
a. a modified CH2 domain that is modified relative to the unmodified CH2 domain to decrease effector function;
b. a modified CH3 domain that is modified relative to the unmodified CH3 domain to lack the homodimerize;
c. a linker sequence; and
d. a transmembrane domain.

10. The polypeptide of any one of claims 1-9, wherein the FcRn binding polypeptide comprises from C-terminus to N-terminus:
a. a modified CH3 domain that is modified relative to the unmodified CH3 domain to lack the homodimerize;
b. a modified CH2 domain that is modified relative to the unmodified CH2 domain to decrease effector function;
c. a linker sequence; and
d. a transmembrane domain.

11. The polypeptide of any one of claims 1-10, wherein the transmembrane domain is a multipass transmembrane domain.

12. The polypeptide of any one of the claims 1-11, further comprising a targeting domain selected from the group consisting of: scFv, (scFv)₂, Fab, Fab′, F(ab′)₂, F(abl)₂, Fv, dAb, Fd fragments, diabodies, F(ab)2, F(ab′), F(ab′)3, Fd, Fv, disulfide linked Fv, dAb, sdAb, nanobody, CDR, di-scFv, bi-scFv, tascFv (tandem scFv), AVIBODY (e.g., diabody, triabody, tetrabody), T-cell engager (BiTE), V-NAR domain, Fcab, IgGACH2, DVD-Ig, probody, intrabody, DARPin, Centyrin, affibody, affilin, affitin, anticalin, avimer, Fynomer, Kunitz domain peptide, monobody, adnectin, tribody, and nanofitin.

13. A nucleic acid encoding the polypeptide of any one of claims 1-12.

14. An expression vector comprising the nucleic acid of claim 13.

15. A cell comprising the nucleic acid of claim 13 or the expression vector of claim 14.

16. An extracellular vesicle comprising the polypeptide of any one of claims 1 to 12.

17. A hybridosome comprising the polypeptide of any one of claims 1 to 12.

18. A method for purifying an extracellular vesicle (EV), wherein said method comprises:
a. providing the EV wherein the EV is associated with a first binding partner, wherein the first binding partner is capable of binding to the Fc binding site of an FcRn in a pH dependent manner; and
b. contacting at a first pH the EV associated with the first binding partner with a second binding partner, wherein the second binding partner comprises the Fc binding site of the FcRn and is associated with a solid matrix; and
c. eluting the EV associated with the first binding partner from the solid matrix at a second pH.

19. The method of claim 18, wherein the method comprises a washing step at the first pH.

20. The method of claim 18 or 19, wherein the first pH is below 6.5.

21. The method of any one of claims 18 to 20, wherein the second pH is above 7.4.

22. A method for purifying an EV, wherein said method comprises:
a. providing the EV wherein the EV is associated with a first binding partner, wherein the first binding partner is capable of binding to the Fc binding site of an FcRn in a pH dependent manner and comprises or consists of the polypeptide of any one of claims 1-12; and
b. contacting at a first pH the EV associated with the first binding partner with a second binding partner, wherein the second binding partner comprises the Fc binding site of the FcRn and is associated with a solid matrix; and
c. eluting the EV associated with the first binding partner from the solid matrix at a second pH.

23. The method of claim 22, wherein the method comprises a washing step at the first pH.

24. The method of claim 22 or 23, wherein the first pH is below 6.5.

25. The method of any one of claims 22 to 24, wherein the second pH is above 7.4.


SEQUENCE LISTING (73 sequences)

SEQ ID NO: 1 - protein, 5 aa, Artificial Sequence
  Linker sequence
  REPEAT (1)..(5): the sequence can be repeated n times, where n can be an integer number from 1 to 10
GGGGS

SEQ ID NO: 2 - protein, 5 aa, Artificial Sequence
  Linker sequence
GGGGS

SEQ ID NO: 3 - protein, 10 aa, Artificial Sequence
  Linker sequence
GGGGSGGGGS

SEQ ID NO: 4 - protein, 15 aa, Artificial Sequence
  Linker sequence
GGGGSGGGGSGGGGS

SEQ ID NO: 5 - protein, 14 aa, Artificial Sequence
  Linker sequence
GGGGSGGGGSGGGG

SEQ ID NO: 6 - protein, 20 aa, Artificial Sequence
  Consensus sequence capable of binding the modified Fc domain of an immunoglobulin (FcRn binding site)
  misc_feature (8)..(8): Xaa can be any naturally occurring amino acid
  misc_feature (10)..(13): Xaa can be any naturally occurring amino acid
  misc_feature (15)..(15): Xaa can be any naturally occurring amino acid
  misc_feature (17)..(17): Xaa can be any naturally occurring amino acid
  misc_feature (19)..(19): Xaa can be any naturally occurring amino acid
LNGEEFMXFXXXXGXWXGXW

SEQ ID NO: 7 - protein, 365 aa, Homo sapiens
  Human FcRn
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 8 - protein, 365 aa, Mouse
  Mouse FcRn
MGMPLPWALSLLLVLLPQTWGSETRPPLMYHLTAVSNPSTGLPSFWATGWLGPQQYLTYNSLRQEADPCGAWMWENQVSWYWEKETTDLKSKEQLFLEALKTLEKILNGTYTLQGLLGCELASDNSSVPTAVFALNGEEFMKFNPRIGNWTGEWPETEIVANLWMKQPDAARKESEFLLNSCPERLLGHLERGRRNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSSARSSVPVVGIVLGLLLVVVAIAGGVLLWGRMRSGLPAPWLSLSGDDSGDLLPGGNLPPEAEPQGANAFPATS

SEQ ID NO: 9 - protein, 217 aa, Artificial Sequence
  Fc domain of IgG1 wt
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 216 aa, Artificial Sequence
  Fc domain of IgG2 wt
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 11 - protein, 216 aa, Artificial Sequence
  Fc domain of IgG3 wt
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 12 - protein, 217 aa, Artificial Sequence
  Fc domain of IgG4 wt
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 217 aa, Artificial Sequence
  modFC_1
APEAAGGPSVFLFPPKPKDTLMISRTPEVTCVVVDVSHEDPEVKFNWYVDGVEVHNAKTKPREEQYGSTYRVVSVLTVLHQDWLNGKEYKCAVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALANHYTQKSLSLSPGK

SEQ ID NO: 14 - protein, 217 aa, Artificial Sequence
  modFC_2
APEAAGGPSVFLFPPKPKDTLMISRTPEVTCVVVDVSHEDPEVKFNWYVDGVEVHNAKTKPREEQYGSTYRVVSVLTVLHQDWLNGKEYKCAVSNKALAAPIEKTISKAKGQPREPQVYTSPPSRDELTKNQVSLRCHVKGFYPSDIAVEWESNGQPENNYKTTKPVLDSDGSFELKSALTVDKSRWQQGNVFSCSVMHEALANHYTQKSLSLSPGK

SEQ ID NO: 15 - protein, 217 aa, Artificial Sequence
  modFC_3
APEAAGGPSVFLFPPKPKDTLMISRTPEVTCVVVDVSHEDPEVKFNWYVDGVEVHNAKTKPREEQYGSTYRVVSVLTVLHQDWLNGKEYKCAVSNKALAAPIEKTISKAKGQPREPQVYTYPPSRDELTKNQVSLYCAVKGFYPSDIAVEWESNGQPENNYKTRPPVLDSDGSFRLMSALTVDKSRWQQGNVFSCSVMHEALANHYTQKSLSLSPGK

SEQ ID NO: 16 - protein, 217 aa, Artificial Sequence
  modFC_4
APEAAGGPSVFLFPPKPKDTLMISRTPEVTCVVVDVSHEDPEVKFNWYVDGVEVHNAKTKPREEQYGSTYRVVSVLTVLHQDWLNGKEYKCAVSNKALAAPIEKTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALANHYTQKSLSLSPGK

SEQ ID NO: 17 - protein, 217 aa, Artificial Sequence
  modFC_5
APELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLSLSPGK

SEQ ID NO: 18 - protein, 217 aa, Artificial Sequence
  modFC_6
APEAAGGPSVFLFPPKPKDTLYITREPEVTCVVVDVSHEDPEVKFNWYVDGVEVHNAKTKPREEQYGSTYRVVSVLTVLHQDWLNGKEYKCAVSNKALAAPIEKTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALANHYTQKSLSLSPGK

SEQ ID NO: 19 - protein, 217 aa, Artificial Sequence
  modFC_7
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLSLSPGK

SEQ ID NO: 20 - protein, 217 aa, Artificial Sequence
  modFC_8
APEAAGGPSVFLFPPKPKDTLYITREPEVTCVVVDVSHEDPEVKFNWYVDGVEVHNAKTKPREEQYGSTYRVVSVLAVLHQDWLNGKEYKCAVSNKALAAPIEKTISKAKGQPREPQVYTSPPSRDELTKNQVSLRCHVKGFYPSDIAVAWESNGQPENNYKTTKPVLDSDGSFFLYSKLTVDKSRWQQGNVFSCSVMHEALAAHYTQKSLSLSPGK

SEQ ID NO: 21 - protein, 217 aa, Artificial Sequence
  modFC_9
APEAAGGPSVFLFPPKPKDTLMISRDPEVTCVVVDVSHEDPEVKFNWYVDGVEVHNAKTKPREEQYGSTYRVVSVLRVLHVDWLNGKEYKCAVSNKALAAPIEKTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALANHYTQKSLSLSPGK

SEQ ID NO: 22 - protein, 217 aa, Artificial Sequence
  modFC_10
APEAAGGPSVFLFPPKPKDTLMISRDPEVTCVVVDVSHEDPEVKFNWYVDGVEVHNAKTKPREEQYGSTYRVVSVLTVLHQDWLDGKEYKCAVSNKALAAPIEKTISKAKGQPREPQVYTSPPSRDELTKNQVSLRCHVKGFYPSDIVVEWESNGQPENNYKTTKPVLDSDGSFFLYSKLTVDKSRWQQGNVFSCSVMHEALANHYTQKSLSLSPGK

SEQ ID NO: 23 - protein, 217 aa, Artificial Sequence
  modFC_11
APEAAGGPSVFLFPPKPKDTLMISRDPEVTCVVVDVSHEDPEVKFNWYVDGVEVHDAKTKPREEQYGSTYRVVSVLRVLHVDWLNGKEYKCAVSNKALAAPIEKTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALANHYTQKSLSLSPGK

SEQ ID NO: 24 - protein, 217 aa, Artificial Sequence
  modFC_12
APEAAGGPSVFLFPPKPKDTLMISRTPEVTCVVVDVSHEDPEVKFNWYVDGVEVNNAKTKPREEQYGSTYRVVSVLQVLHQDWLDGKEYKCAVSNKALAAPIEKTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALANHYTQKSLSLSPGK

SEQ ID NO: 25 - protein, 217 aa, Artificial Sequence
  modFC_13
APEAAGGPSVFLFPPKPKDTLMISRDPEVTCVVVDVSHEDPEVKFNWYVDGVEVHNAKTKPREEQYGSTYRVVSVLRVLHVDWLNGKEYKCAVSNKALAAPIEKTISKAKGQPREPQVYTSPPSRDELTKNQVSLRCHVKGFYPSDIVVEWESNGQPENNYKTTKPVLDSDGSFFLYSKLTVDKSRWQQGNVFSCSVMHEALANHYTQKSLSLSPGK

SEQ ID NO: 26 - protein, 217 aa, Artificial Sequence
  modFC_14
APEAAGGPSVFLFPPKPKDTLMISRTPEVTCVVVDVSHEDPEVKFNWYVDGVEVDNAKTKPREEQYGSTYRVVSVLTVLHVDWLNGKEYKCAVSNKALAAPIEKTISKAKGQPREPQVYTSPPSRDELTKNQVSLRCHVKGFYPSDIVVEWESNGQPENNYKTTKPVLDSDGSFFLYSKLTVDKSRWQQGNVFSCSVMHEALANHYTQKSLSLSPGK

SEQ ID NO: 27 - protein, 217 aa, Artificial Sequence
  modFC_15
APEAAGGPSVFLFPPKPKDTLMISRDPEVTCVVVDVSHEDPEVKFNWYVDGVEVDNAKTKPREEQYGSTYRVVSVLTVLHQDWLNGKEYKCAVSNKALAAPIEKTISKAKGQPREPQVYTSPPSRDELTKNQVSLRCHVKGFYPSDIVVEWESNGQPENNYKTTKPVLDSDGSFFLYSKLTVDKSRWQQGNVFSCSVMHEALANHYTQKSLSLSPGK

SEQ ID NO: 28 - protein, 217 aa, Artificial Sequence
  modFC_16
APEAAGGPSVFLFPPKPKDTLMISRDPEVTCVVVDVSHEDPEVKFNWYVDGVEVHNAKTKPREEQYGSTYRVVSVLTVLHVDWLNGKEYKCAVSNKALAAPIEKTISKAKGQPREPQVYTSPPSRDELTKNQVSLRCHVKGFYPSDIVVEWESNGQPENNYKTTKPVLDSDGSFFLYSKLTVDKSRWQQGNVFSCSVMHEALANHYTQKSLSLSPGK

SEQ ID NO: 29 - protein, 217 aa, Artificial Sequence
  modFC_17
APEAAGGPSVFLFPPKPKDTLMISRDPEVTCVVVDVSHEDPEVKFNWYVDGVEVDDAKTKPREEQYGSTYRVVSVLRVLHQDWLNGKEYKCAVSNKALAAPIEKTISKAKGQPREPQVYTSPPSRDELTKNQVSLRCHVKGFYPSDIVVEWESNGQPENNYKTTKPVLDSDGSFFLYSKLTVDKSRWQQGNVFSCSVMHEALANHYTQKSLSLSPGK

SEQ ID NO: 30 - protein, 217 aa, Artificial Sequence
  modFC_18
APEAAGGPSVFLFPPKPKDTLMISRDPEVTCVVVDVSHEDPEVKFNWYVDGVEVDNAKTKPREEQYGSTYRVVSVLRVLHVDWLNGKEYKCAVSNKALAAPIEKTISKAKGQPREPQVYTSPPSRDELTKNQVSLRCHVKGFYPSDIVVEWESNGQPENNYKTTKPVLDSDGSFFLYSKLTVDKSRWQQGNVFSCSVMHEALANHYTQKSLSLSPGK

SEQ ID NO: 31 - protein, 217 aa, Artificial Sequence
  modFC_19
APEAAGGPSVFLFPPKPKDTLMISRTPEVTCVVVDVSHEDPEVKFNWYVDGVEVHNAKTKPREEQYGSTYRVVSVLQVLHVDWLNGKEYKCAVSNKALAAPIEKTISKAKGQPREPQVYTSPPSRDELTKNQVSLRCHVKGFYPSDIVVEWESNGQPENNYKTTKPVLDSDGSFFLYSKLTVDKSRWQQGNVFSCSVMHEALANHYTQKSLSLSPGK

SEQ ID NO: 32 - protein, 217 aa, Artificial Sequence
  modFC_20
APEAAGGPSVFLFPPKPKDTLMISRTPEVTCVVVDVSHEDPEVKFNWYVDGVEVDNAKTKPREEQYGSTYRVVSVLQVLHQDWLNGKEYKCAVSNKALAAPIEKTISKAKGQPREPQVYTSPPSRDELTKNQVSLRCHVKGFYPSDIVVEWESNGQPENNYKTTKPVLDSDGSFFLYSKLTVDKSRWQQGNVFSCSVMHEALANHYTQKSLSLSPGK

SEQ ID NO: 33 - protein, 217 aa, Artificial Sequence; identical to SEQ ID NO: 30
  modFC_21

SEQ ID NO: 34 - protein, 217 aa, Artificial Sequence
  modFC_22
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKSPSRDELTKNQVSLSCHVKGFYPSDIAVLWESYGTEWSSYKTTVPVLDSDGSFRLASYLTVTKEEWQQGFVFSCSVMHEALHNHYTQKSLSLSPGK

SEQ ID NO: 35 - protein, 26 aa, Artificial Sequence
  transmembrane domain of a membrane bound IgG
GLWTTITIFITLFLLSVCYSATVTFF

SEQ ID NO: 36 - protein, 21 aa, Artificial Sequence
  EPHA1 (P21709)(a.a. positions 548-568)
IVAVIFGLLLGAALLLGILVF

SEQ ID NO: 37 - protein, 21 aa, Artificial Sequence
  EPHA2 (P29317)(a.a. positions 538-558)
IGGVAVGVVLLLVLAGVGFFI

SEQ ID NO: 38 - protein, 24 aa, Artificial Sequence
  EPHA3 (P29320)(a.a. positions 542-565)
VVMIAISAAVAIILLTVVIYVLIG

SEQ ID NO: 39 - protein, 22 aa, Artificial Sequence
  EPHA4 (P54764)(a.a. positions 548-569)
VLLVSVSGSVVLVVILIAAFVI

SEQ ID NO: 40 - protein, 21 aa, Artificial Sequence
  EPHA5 (P54756)(a.a. positions 574-594)
VIAVSVTVGVILLAVVIGVLL

SEQ ID NO: 41 - protein, 21 aa, Artificial Sequence
  EPHA6 (Q9UF33)(a.a. positions 551-571)
IATAAVGGFTLLVILTLFFLI

SEQ ID NO: 42 - protein, 21 aa, Artificial Sequence
  EPHA7 (Q15375)(a.a. positions 556-576)
IIIAVVAVAGTIILVFMVFGF

SEQ ID NO: 43 - protein, 21 aa, Artificial Sequence
  EPHA8 (P29322)(a.a. positions 543-563)
IVWICLTLITGLVVLLLLLIC

SEQ ID NO: 44 - protein, 21 aa, Artificial Sequence
  EPHA10 (Q5JZY3)(a.a. positions 566-586)
IVVTVVTISALLVLGSVMSVL

SEQ ID NO: 45 - protein, 23 aa, Artificial Sequence
  EPHB1 (P54762)(a.a. positions 541-563)
LIAGSAAAGVVFVVSLVAISIVC

SEQ ID NO: 46 - protein, 21 aa, Artificial Sequence
  EPHB2 (P29323)(a.a. positions 544-564)
IIGSSAAGLVFLIAVVVIAIV

SEQ ID NO: 47 - protein, 21 aa, Artificial Sequence
  EPHB3 (P54753)(a.a. positions 560-580)
IVGSATAGLVFVVAVVVIAIV

SEQ ID NO: 48 - protein, 21 aa, Artificial Sequence
  EPHB4 (P54760)(a.a. positions 540-560)
LIAGTAVVGVVLVLVVIVVAV

SEQ ID NO: 49 - protein, 21 aa, Artificial Sequence
  EPHB6 (O15197)(a.a. positions 595-615)
LVIGSILGALAFLLLAAITVL

SEQ ID NO: 50 - protein, 21 aa, Artificial Sequence
  FPRP(a.a. positions 833-853)
LLIGVGLSTVIGLLSCLIGYC

SEQ ID NO: 51 - protein, 570 aa, Artificial Sequence
  EPHA1 (27-596)
EVTLMDTSKAQGELGWLLDPPKDGWSEQQQILNGTPLYMYQDCPMQGRRDTDHWLRSNWIYRGEEASRVHVELQFTVRDCKSFPGGAGPLGCKETFNLLYMESDQDVGIQLRRPLFQKVTTVAADQSFTIRDLVSGSVKLNVERCSLGRLTRRGLYLAFHNPGACVALVSVRVFYQRCPETLNGLAQFPDTLPGPAGLVEVAGTCLPHARASPRPSGAPRMHCSPDGEWLVPVGRCHCEPGYEEGGSGEACVACPSGSYRMDMDTPHCLTCPQQSTAESEGATICTCESGHYRAPGEGPQVACTGPPSAPRNLSFSASGTQLSLRWEPPADTGGRQDVRYSVRCSQCQGTAQDGGPCQPCGVGVHFSPGARGLTTPAVHVNGLEPYANYTFNVEAQNGVSGLGSSGHASTSVSISMGHAESLSGLSLRLVKKEPRQLELTWAGSRPRSPGANLTYELHVLNQDEERYQMVLEPRVLLTELQPDTTYIVRVRMLTPLGPGPFSPDHEFRTSPPVSRGLTGGEIVAVIFGLLLGAALLLGILVFRSRRAQRQRQQRQRDRATDVDREDKLWL

SEQ ID NO: 52 - protein, 558 aa, Artificial Sequence
  EPHA2 (28-585)
EVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPL

SEQ ID NO: 53 - protein, 562 aa, Artificial Sequence
  EPHA3 (29-590)
EVNLLDSKTIQGELGWISYPSHGWEEISGVDEHYTPIRTYQVCNVMDHSQNNWLRTNWVPRNSAQKIYVELKFTLRDCNSIPLVLGTCKETFNLYYMESDDDHGVKFREHQFTKIDTIAADESFTQMDLGDRILKLNTEIREVGPVNKKGFYLAFQDVGACVALVSVRVYFKKCPFTVKNLAMFPDTVPMDSQSLVEVRGSCVNNSKEEDPPRMYCSTEGEWLVPIGKCSCNAGYEERGFMCQACRPGFYKALDGNMKCAKCPPHSSTQEDGSMNCRCENNYFRADKDPPSMACTRPPSSPRNVISNINETSVILDWSWPLDTGGRKDVTFNIICKKCGWNIKQCEPCSPNVRFLPRQFGLTNTTVTVTDLLAHTNYTFEIDAVNGVSELSSPPRQFAAVSITTNQAAPSPVLTIKKDRTSRNSISLSWQEPEHPNGIILDYEVKYYEKQEQETSYTILRARGTNVTISSLKPDTIYVFQIRARTAAGYGTNSRKFEFETSPDSFSISGESSQVVMIAISAAVAIILLTVVIYVLIGRFCGYKSKHGADEKRLHFGNGHLKL

SEQ ID NO: 54 - protein, 561 aa, Artificial Sequence
  EPHA4 (30-590)
EVTLLDSRSVQGELGWIASPLEGGWEEVSIMDEKNTPIRTYQVCNVMEPSQNNWLRTDWITREGAQRVYIEIKFTLRDCNSLPGVMGTCKETFNLYYYESDNDKERFIRENQFVKIDTIAADESFTQVDIGDRIMKLNTEIRDVGPLSKKGFYLAFQDVGACIALVSVRVFYKKCPLTVRNLAQFPDTITGADTSSLVEVRGSCVNNSEEKDVPKMYCGADGEWLVPIGNCLCNAGHEERSGECQACKIGYYKALSTDATCAKCPPHSYSVWEGATSCTCDRGFFRADNDAASMPCTRPPSAPLNLISNVNETSVNLEWSSPQNTGGRQDISYNVVCKKCGAGDPSKCRPCGSGVHYTPQQNGLKTTKVSITDLLAHTNYTFEIWAVNGVSKYNPNPDQSVSVTVTTNQAAPSSIALVQAKEVTRYSVALAWLEPDRPNGVILEYEVKYYEKDQNERSYRIVRTAARNTDIKGLNPLTSYVFHVRARTAAGYGDFSEPLEVTTNTVPSRIIGDGANSTVLLVSVSGSVVLVVILIAAFVISRRRSKYSKAKQEADEEKHLN

SEQ ID NO: 55 - protein, 560 aa, Artificial Sequence
  EPHA5 (60-619)
EVNLLDSRTVMGDLGWIAFPKNGWEEIGEVDENYAPIHTYQVCKVMEQNQNNWLLTSWISNEGASRIFIELKFTLRDCNSLPGGLGTCKETFNMYYFESDDQNGRNIKENQYIKIDTIAADESFTELDLGDRVMKLNTEVRDVGPLSKKGFYLAFQDVGACIALVSVRVYYKKCPSVVRHLAVFPDTITGADSSQLLEVSGSCVNHSVTDEPPKMHCSAEGEWLVPIGKCMCKAGYEEKNGTCQVCRPGFFKASPHIQSCGKCPPHSYTHEEASTSCVCEKDYFRRESDPPTMACTRPPSAPRNAISNVNETSVFLEWIPPADTGGRKDVSYYIACKKCNSHAGVCEECGGHVRYLPRQSGLKNTSVMMVDLLAHTNYTFEIEAVNGVSDLSPGARQYVSVNVTTNQAAPSPVTNVKKGKIAKNSISLSWQEPDRPNGIILEYEIKYFEKDQETSYTIIKSKETTITAEGLKPASVYVFQIRARTAAGYGVFSRRFEFETTPVFAASSDQSQIPVIAVSVTVGVILLAVVIGVLLSGSCCECGCGRASSLCAVAHPSLIW

SEQ ID NO: 56 - protein, 565 aa, Artificial Sequence
  EPHA6 (34-589)
QVVLLDTTTVLGELGWKTYPLNGWDAITEMDEHNRPIHTYQVCNVMEPNQNNWLRTNWISRDAAQKIYVEMKFTLRDCNSIPWVLGTCKETFNLFYMESDESHGIKFKPNQYTKIDTIAADESFTQMDLGDRILKLNTEIREVGPIERKGFYLAFQDIGACIALVSVRVFYKKCPFTVRNLAMFPDTIPRVDSSSLVEVRGSCVKSAEERDTPKLYCGADGDWLVPLGRCICSTGYEEIEGSCHACRPGFYKAFAGNTKCSKCPPHSLTYMEATSVCQCEKGYFRAEKDPPSMACTRPPSAPRNVVFNINETALILEWSPPSDTGGRKDLTYSVICKKCGLDTSQCEDCGGGLRFIPRHTGLINNSVIVLDFVSHVNYTFEIEAMNGVSELSFSPKPFTAITVTTDQDAPSLIGVVRKDWASQNSIALSWQAPAFSNGAILDYEIKYYEKEHEQLTYSSTRSKAPSVIITGLKPATKYVFHIRVRTATGYSGYSQKFEFETGDETSDMAAEQGQILVIATAAVGGFTLLVILTLFFLITGRCQWYIKAKMKSEEKRRNHLQNGHL

SEQ ID NO: 57 - protein, 578 aa, Artificial Sequence
  EPHA7 (30-607)
AKEVLLLDSKAQQTELEWISSPPNGWEEISGLDENYTPIRTYQVCQVMEPNQNNWLRTNWISKGNAQRIFVELKFTLRDCNSLPGVLGTCKETFNLYYYETDYDTGRNIRENLYVKIDTIAADESFTQGDLGERKMKLNTEVREIGPLSKKGFYLAFQDVGACIALVSVKVYYKKCWSIIENLAIFPDTVTGSEFSSLVEVRGTCVSSAEEEAENAPRMHCSAEGEWLVPIGKCICKAGYQQKGDTCEPCGRGFYKSSSQDLQCSRCPTHSFSDKEGSSRCECEDGYYRAPSDPPYVACTRPPSAPQNLIFNINQTTVSLEWSPPADNGGRNDVTYRILCKRCSWEQGECVPCGSNIGYMPQQTGLEDNYVTVMDLLAHANYTFEVEAVNGVSDLSRSQRLFAAVSITTGQAAPSQVSGVMKERVLQRSVELSWQEPEHPNGVITEYEIKYYEKDQRERTYSTVKTKSTSASINNLKPGTVYVFQIRAFTAAGYGNYSPRLDVATLEEATGKMFEATAVSSEQNPVIIIAVVAVAGTIILVFMVFGFIIGRRHCGYSKADQEGDEELYFHFKFPGTKT

SEQ ID NO: 58 - protein, 559 aa, Artificial Sequence
  EPHA8 (31-589)
EVNLLDTSTIHGDWGWLTYPAHGWDSINEVDESFQPIHTYQVCNVMSPNQNNWLRTSWVPRDGARRVYAEIKFTLRDCNSMPGVLGTCKETFNLYYLESDRDLGASTQESQFLKIDTIAADESFTGADLGVRRLKLNTEVRSVGPLSKRGFYLAFQDIGACLAILSLRIYYKKCPAMVRNLAAFSEAVTGADSSSLVEVRGQCVRHSEERDTPKMYCSAEGEWLVPIGKCVCSAGYEERRDACVACELGFYKSAPGDQLCARCPPHSHSAAPAAQACHCDLSYYRAALDPPSSACTRPPSAPVNLISSVNGTSVTLEWAPPLDPGGRSDITYNAVCRRCPWALSRCEACGSGTRFVPQQTSLVQASLLVANLLAHMNYSFWIEAVNGVSDLSPEPRRAAVVNITTNQAAPSQVVVIRQERAGQTSVSLLWQEPEQPNGIILEYEIKYYEKDKEMQSYSTLKAVTTRATVSGLKPGTRYVFQVRARTSAGCGRFSQAMEVETGKPRPRYDTRTIVWICLTLITGLVVLLLLLICKKRHCGYSKAFQDSDEEKMHYQNGQA

SEQ ID NO: 59 - protein, 570 aa, Artificial Sequence
  EPHA10 (35-604)
EVILLDSKASQAELGWTALPSNGWEEISGVDEHDRPIRTYQVCNVLEPNQDNWLQTGWISRGRGQRIFVELQFTLRDCSSIPGAAGTCKETFNVYYLETEADLGRGRPRLGGSRPRKIDTIAADESFTQGDLGERKMKLNTEVREIGPLSRRGFHLAFQDVGACVALVSVRVYYKQCRATVRGLATFPATAAESAFSTLVEVAGTCVAHSEGEPGSPPRMHCGADGEWLVPVGRCSCSAGFQERGDFCEACPPGFYKVSPRRPLCSPCPEHSRALENASTFCVCQDSYARSPTDPPSASCTRPPSAPRDLQYSLSRSPLVLRLRWLPPADSGGRSDVTYSLLCLRCGREGPAGACEPCGPRVAFLPRQAGLRERAATLLHLRPGARYTVRVAALNGVSGPAAAAGTTYAQVTVSTGPGAPWEEDEIRRDRVEPQSVSLSWREPIPAGAPGANDTEYEIRYYEKGQSEQTYSMVKTGAPTVTVTNLKPATRYVFQIRAASPGPSWEAQSFNPSIEVQTLGEAASGSRDQSPAIVVTVVTISALLVLGSVMSVLAIWRRPCSYGKGGGDAHD

SEQ ID NO: 60 - protein, 572 aa, Artificial Sequence
  EPHB1 (21-591)
ETLMDTRTATAELGWTANPASGWEEVSGYDENLNTIRTYQVCNVFEPNQNNWLLTTFINRRGAHRIYTEMRFTVRDCSSLPNVPGSCKETFNLYYYETDSVIATKKSAFWSEAPYLKVDTIAADESFSQVDFGGRLMKVNTEVRSFGPLTRNGFYLAFQDYGACMSLLSVRVFFKKCPSIVQNFAVFPETMTGAESTSLVIARGTCIPNAEEVDVPIKLYCNGDGEWMVPIGRCTCKPGYEPENSVACKACPAGTFKASQEAEGCSHCPSNSRSPAEASPICTCRTGYYRADFDPPEVACTSVPSGPRNVISIVNETSIILEWHPPRETGGRDDVTYNIICKKCRADRRSCSRCDDNVEFVPRQLGLTECRVSISSLWAHTPYTFDIQAINGVSSKSPFPPQHVSVNITTNQAAPSTVPIMHQVSATMRSITLSWPQPEQPNGIILDYEIRYYEKEHNEFNSSMARSQTNTARIDGLRPGMVYVVQVRARTVAGYGKFSGKMCFQTLTDDDYKSELREQLPLIAGSAAAGVVFVVSLVAISIVCSRKRAYSKEAVYSDKLQHYSTGRGSPGM

SEQ ID NO: 61 - protein, 571 aa, Artificial Sequence
  EPHB2 (19-589)
VEETLMDSTTATAELGWMVHPPSGWEEVSGYDENMNTIRTYQVCNVFESSQNNWLRTKFIRRRGAHRIHVEMKFSVRDCSSIPSVPGSCKETFNLYYYEADFDSATKTFPNWMENPWVKVDTIAADESFSQVDLGGRVMKINTEVRSFGPVSRSGFYLAFQDYGGCMSLIAVRVFYRKCPRIIQNGAIFQETLSGAESTSLVAARGSCIANAEEVDVPIKLYCNGDGEWLVPIGRCMCKAGFEAVENGTVCRGCPSGTFKANQGDEACTHCPINSRTTSEGATNCVCRNGYYRADLDPLDMPCTTIPSAPQAVISSVNETSLMLEWTPPRDSGGREDLVYNIICKSCGSGRGACTRCGDNVQYAPRQLGLTEPRIYISDLLAHTQYTFEIQAVNGVTDQSPFSPQFASVNITTNQAAPSAVSIMHQVSRTVDSITLSWSQPDQPNGVILDYELQYYEKELSEYNATAIKSPTNTVTVQGLKAGAIYVFQVRARTVAGYGRYSGKMYFQTMTEAEYQTSIQEKLPLIIGSSAAGLVFLIAVVVIAIVCNRRGFERADSEYTDKLQHYTSGHM

SEQ ID NO: 62 - protein, 567 aa, Artificial Sequence
  EPHB3 (39-605)
EETLMDTKWVTSELAWTSHPESGWEEVSGYDEAMNPIRTYQVCNVRESSQNNWLRTGFIWRRDVQRVYVELKFTVRDCNSIPNIPGSCKETFNLFYYEADSDVASASSPFWMENPYVKVDTIAPDESFSRLDAGRVNTKVRSFGPLSKAGFYLAFQDQGACMSLISVRAFYKKCASTTAGFALFPETLTGAEPTSLVIAPGTCIPNAVEVSVPLKLYCNGDGEWMVPVGACTCATGHEPAAKESQCRPCPPGSYKAKQGEGPCLPCPPNSRTTSPAASICTCHNNFYRADSDSADSACTTVPSPPRGVISNVNETSLILEWSEPRDLGGRDDLLYNVICKKCHGAGGASACSRCDDNVEFVPRQLGLTERRVHISHLLAHTRYTFEVQAVNGVSGKSPLPPRYAAVNITTNQAAPSEVPTLRLHSSSGSSLTLSWAPPERPNGVILDYEMKYFEKSEGIASTVTSQMNSVQLDGLRPDARYVVQVRARTVAGYGQYSRPAEFETTSERGSGAQQLQEQLPLIVGSATAGLVFVVAVVVIAIVCLRKQRHGSDSEYTEKLQQYIAPGM

SEQ ID NO: 63 - protein, 567 aa, Artificial Sequence
  EPHB4 (19-583)
EETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLALIAGTAVVGVVLVLVVIVVAVLCLRKQSNGREAEYSDKHGQYLI

SEQ ID NO: 64 - protein, 617 aa, Artificial Sequence
  EPHB6 (33-649)
EEVLLDTTGETSEIGWLTYPPGGWDEVSVLDDQRRLTRTFEACHVAGAPPGTGQDNWLQTHFVERRGAQRAHIRLHFSVRACSSLGVSGGTCRETFTLYYRQAEEPDSPDSVSSWHLKRWTKVDTIAADESFPSSSSSSSSSSSAAWAVGPHGAGQRAGLQLNVKERSFGPLTQRGFYVAFQDTGACLALVAVRLFSYTCPAVLRSFASFPETQASGAGGASLVAAVGTCVAHAEPEEDGVGGQAGGSPPRLHCNGEGKWMVAVGGCRCQPGYQPARGDKACQACPRGLYKSSAGNAPCSPCPARSHAPNPAAPVCPCLEGFYRASSDPPEAPCTGPPSAPQELWFEVQGSALMLHWRLPRELGGRGDLLFNVVCKECEGRQEPASGGGGTCHRCRDEVHFDPRQRGLTESRVLVGGLRAHVPYILEVQAVNGVSELSPDPPQAAAINVSTSHEVPSAVPVVHQVSRASNSITVSWPQPDQTNGNILDYQLRYYDQAEDESHSFTLTSETNTATVTQLSPGHIYGFQVRARTAAGHGPYGGKVYFQTLPQGELSSQLPERLSLVIGSILGALAFLLLAAITVLAVVFQRKRRGTGYTEQLQQYSSPGLGVKYYIDPS

SEQ ID NO: 65 - protein, 517 aa, Artificial Sequence
  AT1B3 - L - monoFC
MTKNEKKSLNQSLAEWKLFIYNPTTGEFLGRTAKSWGLILLFYLVFYGFLAALFSFTMWVMLQTLNDEVPKYRDQIPSPGLMVFPKPVTALEYTFSRSDPTSYAGYIEDLKKFLKPYTLEEQKNLTVCPDGALFEQKGPVYVACQFPISLLQACSGMNDPDFGYSQGNPCILVKMNRIIGLKPEGVPRIDCVSKNEDIPNVAVYPHNGMIDLKYFPYYGKKLHVGYLQPLVAVQVSFAPNNTGKEVTVECKIDGSANLKSQDDRDKFLGRVMFKITARAGGGSGGGGSGGGGSGGGGSGGAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKSPSRDELTKNQVSLSCHVKGFYPSDIAVLWESYGTEWSSYKTTVPVLDSDGSFRLASYLTVTKEEWQQGFVFSCSVMHEALHNHYTQKSLSLSPGK

SEQ ID NO: 66 - protein, 545 aa, Artificial Sequence
  Zip2 + L + C-terminal monoFC
MEQLLGIKLGCLFALLALTLGCGLTPICFKWFQIDAARGHHRLVLRLLGCISAGVFLGAGFMAMTAEALEEIESQIQKFMVQNRSASERNSSGDADSAHMEYPYGELIISLGFFFVFFLESLALQCCPGAAGGSTVQDEEWGGAHIFELHSHGHLPSPSKGPLRALVLLLSLSFHSVFEGLAVGLQPTVAATVQLCLAVLAHKGLVVFGVGMRLVHLGTSSRWAVFSILLLALMSPLGLAVGLAVTGGDSEGGRGLAQAVLEGVAAGTFLYVTFLEILPRELASPEAPLAKWSCVAAGFAFMAFIALWAGGGSGGGGSGGGGSGGGGSGGAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSP

SEQ ID NO: 67 - protein, 545 aa, Artificial Sequence
  N-terminal monoFC + L + Zip2
MAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGGGSGGGGSGGGGSGGGGSGGEQLLGIKLGCLFALLALTLGCGLTPICFKWFQIDAARGHHRLVLRLLGCISAGVFLGAGFMAMTAEALEEIESQIQKFMVQNRSASERNSSGDADSAHMEYPYGELIISLGFFFVFFLESLALQCCPGAAGGSTVQDEEWGGAHIFELHSHGHLPSPSKGPLRALVLLLSLSFHSVFEGLAVGLQPTVAATVQLCLAVLAHKGLVVFGVGMRLVHLGTSSRWAVFSILLLALMSPLGLAVGLAVTGGDSEGGRGLAQAVLEGVAAGTFLYVTFLEILPRELASPEAPLAKWSCVAAGFAFMAFIALWA

SEQ ID NO: 68 - protein, 781 aa, Artificial Sequence
  N-terminal monoFC + L + Zip2 + L + C-terminal monoFc
MAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGGGSGGGGSGGGGSGGGGSGGEQLLGIKLGCLFALLALTLGCGLTPICFKWFQIDAARGHHRLVLRLLGCISAGVFLGAGFMAMTAEALEEIESQIQKFMVQNRSASERNSSGDADSAHMEYPYGELIISLGFFFVFFLESLALQCCPGAAGGSTVQDEEWGGAHIFELHSHGHLPSPSKGPLRALVLLLSLSFHSVFEGLAVGLQPTVAATVQLCLAVLAHKGLVVFGVGMRLVHLGTSSRWAVFSILLLALMSPLGLAVGLAVTGGDSEGGRGLAQAVLEGVAAGTFLYVTFLEILPRELASPEAPLAKWSCVAAGFAFMAFIALWAGGGSGGGGSGGGGSGGGGSGGAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSP

SEQ ID NO: 69 - protein, 273 aa, Artificial Sequence
  Soluble hFcRn
SAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAK

SEQ ID NO: 70 - protein, 140 aa, Artificial Sequence
  Soluble b2m
METDTLLLWVLLLWVPGSTGDAAQPARRAVRSLVPSSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 71 - protein, 427 aa, Homo spiens
  Human scFcRn
METDTLLLWVLLLWVPGSTGDAAQPARRAVRSLVPSSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAK

SEQ ID NO: 72 - protein, 428 aa, Mouse
  Mouse scFcRn
METDTLLLWVLLLWVPGSTGDAAQPARRAVRSLVPSSGLYAIQKTPQIQVYSRHPPENGKPNILNCYVTQFHPPHIEIQMLENGKKIPKVEMSDMSFSKDWSFYILAHTEFTPTETDTYACRVKHASMAEPKTVYWDRDMGGGGSGGGGSGGGGSRPPLMYHLTAVSNPSTGLPSFWATGWLGPQQYLTYNSLRQEADPCGAWMWENQVSWYWEKETTDLKSKEQLFLEALKTLEKILNGTYTLQGLLGCELASDNSSVPTAVFALNGEEFMKFNPRIGNWTGEWPETEIVANLWMKQPDAARKESEFLLNSCPERLLGHLERGRRNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSSARSS

SEQ ID NO: 73 - protein, 1211 aa, Artificial Sequence
  sequence of full fusion protein 3
MALRRLGAALLLLPLLAAVSDVPRDLEVVAATPTSLLISWYYPFCAFYYRITYGETGGNSPVQEFTVPRSPDTATISGLKPGVDYTITVYAVTCLGSYSRPISINYRTGGGGSGGGGSGGAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGGGSGGGSGGGSGGGSRKCPRIIQNGAIFQETLSGAESTSLVAARGSCIANAEEVDVPIKLYCNGDGEWLVPIGRCMCKAGFEAVENGTVCRGCPSGTFKANQGDEACTHCPINSRTTSEGATNCVCRNGYYRADLDPLDMPCTTIPSAPQAVISSVNETSLMLEWTPPRDSGGREDAVYNIICKSCGSGRGACTRCGDNVQYAPRQLGLTEPRIYASDLLAHTQYTFEIQAVNGVTDQSPFSPQFASVNITTNQAAPSAVSIMHQVSRTVDSITLSWSQPDQPNGVILDYELQYYEKELSEYNATAIKSPTNTVTVQGLKAGAIYVFQVRARTVAGYGRYSGKMYFQTMTEAEYQTEIQEKLPLIIGSSAAGLVFLIAVVVISIVCNRRGFERADSEYTDKLQHYTSGHMTPGMKIYIDPFTYEDPNEAVREFAKEIDISCVKIEQVIGAGEFGEVCSGHLKLPGKREIFVAIKTLKSGYTEKQRRDFLSEASIMGQFDHPNVIHLEGVVTKSTPVMIITEFMENGSLDSFLRQNDGQFTVIQLVGMLRGIAAGMKYLADMNYVHRDLAARNILVNSNLVCKVSDFGLSRFLEDDTSDPTYTSALGGKIPIRWTAPEAIQYRKFTSASDVWSYGIVMWEVMSFGERPYWDMTNQDVINAIEQDYRLPPPMDCPSALHQLMLDCWQKDRNHRPKFGQIVNTLDKMIRNPNSLKAMAPLSSGINLPLGGGEAEAERGKLPGKKLPLEVLIELEANARKAGCTRGCLICLSKIKCTAKMKKYIPGRCADYGGDKKTGQAGIVGAIVDIPEISGFKEMEPMEQFIAQVDRCADCTTGCLKGLANVKCSDLLKKWLPGRCATFADKIQSEVDNIKGLAGD